(19) United States
(12) Patent Application Publication
(10) Pub. No.: US 2025/0353925 A1
(43) Pub. Date: Nov. 20, 2025

(54) CORONAVIRUS-INHIBITING ANTIBODIES

(71) Applicant: THE ROCKEFELLER UNIVERSITY, New York, NY (US)

(72) Inventors: Paul BIENIASZ, New York, NY (US); Theodora HATZIIOANNOU, New York, NY (US); Fengwen ZHANG, New York, NY (US); Ivo LORENZ, Brooklyn, NY (US); Elisabeth NYAKATURA, Jersey City, NJ (US)

(21) Appl. No.: 18/871,290

(22) PCT Filed: Jun. 2, 2023
(86) PCT No.: PCT/US2023/067803

§ 371 Date: Dec. 3, 2024

Related U.S. Application Data
(60) Provisional application No. 63/348,585, filed on Jun. 3, 2022.

(51) Int. Cl. C07K 16/28; A61K 39/00; A61P 37/04; C07K 16/40

(52) U.S. Cl. CPC C07K 16/2896; A61P 37/04; C07K 16/40; A61K 2039/505; C07K 2317/24; C07K 2317/76; C07K 2317/92; C07K 2317/94

(57) ABSTRACT

The current disclosure is directed to antibodies which inhibit coronaviruses, methods of making such antibodies, and the uses of such antibodies for the treatment and prevention of infection caused by coronaviruses.

### CROSS REFERENCE TO RELATED APPLICATION

This application claims the benefit of U.S. Provisional Patent Application No. 63/348,585, filed Jun. 3, 2022, the contents of which are incorporated herein by reference in its entirety.

### INCORPORATION BY REFERENCE OF SEQUENCE LISTING

The Sequence Listing in an XML file, named as 40755WO_SequenceListing.xml of 235 KB, created on May 31, 2023, and submitted to the United States Patent and Trademark Office via Patent Center, is incorporated herein by reference.

### BACKGROUND

Coronaviruses are widespread in the animal kingdom, and frequently cross species barriers. Indeed, seasonal coronaviruses arose from animal reservoirs and cause common colds in humans but are only rarely associated with serious disease. However, in the past 17 years, three coronaviruses (SARS-CoV, MERS and SARS-CoV-2) viruses have emerged that cause severe acute respiratory syndrome in humans. In one case (SARS-CoV-2) a devastating pandemic has ensued, causing millions of deaths and illnesses and global economic disruption.

Bats harbor diverse mammalian coronaviruses and constitute an animal reservoir of numerous SARS-CoV and SARS-CoV-2-like viruses. The diversity and biology of bats offers prime conditions for virus transmission and recombination, the generation of genetic diversity and opportunities for transmission and adaptation to new mammalian hosts. There are now repeated examples of cross-species transmission of bat coronaviruses to other species including humans. This fact, plus the existence of large numbers of diverse and largely uncharacterized bat coronaviruses, indicates a high probability of future epidemics and the need to develop interventions that will target a broad range of coronaviruses.

Many academic laboratories and commercial entities have identified SARS-CoV-2 specific neutralizing monoclonal antibodies for the treatment and prophylaxis of SARS-CoV-2 infection. However, under laboratory conditions and in the natural epidemic, we and others have found that escape mutations to individual human monoclonal antibodies are readily generated, and many of the commercialized anti-SARS-CoV-2 therapeutic antibodies have been rendered obsolete by the emergence of SARS-CoV-2 variants such as omicron.

Despite sharing structural features including the overall folding of the spike protein and the receptor binding domain (RBD), the spike proteins of sarbecoviruses can vary by >25% of amino acid residues. Consequently, there is, in most cases, little or no cross-neutralization activity for monoclonal antibodies raised against an individual virus. Thus, it will likely be difficult, and perhaps impossible, to define spike-binding monoclonal antibodies that potently neutralize SARS-CoV, SARS-CoV-2, neutralizing antibody resistant mutants thereof, as well as the plethora of SARS-related coronaviruses that exist naturally in bats and other mammalian species.

### SUMMARY

All SARS-related coronaviruses in bats and other mammals use Angiotensin-converting enzyme-2 (ACE2) as a receptor. This disclosure describes monoclonal antibodies targeting ACE2, rather than the viral spike proteins, as broad coronavirus inhibitors. Such inhibitors will serve not only to combat the current SARS-CoV-2 pandemic but will be a first line of defense against future coronavirus pandemics.

As an exemplary illustration, purified soluble recombinant forms of the monomeric (8×His tagged) and dimeric (Fc-fused) human ACE2 receptor extracellular domain (residues 1-740) were developed and expressed in Expi293 cells. ACE2 is natively a homodimer, and this conformation was recapitulated by fusing the extracellular domain of human ACE2 to the Fc portion of human IgG1 in the ACE2-Fc fusion protein. AlivaMab mice were immunized with monomeric or dimeric ACE2 and hybridoma supernatants were tested and screened for antibodies that could potently inhibit infection by SARS-CoV-2. Variable domains from these chimeric antibodies were sequenced and combined with human IgG1 Fc domain to generate fully human anti-ACE2 antibodies.

In one aspect, the present disclosure provides anti-angiotensin converting enzyme 2 (ACE2) antibodies and antigen-binding fragments thereof.

One embodiment of the disclosure is directed to an isolated antibody or antigen binding fragment thereof, wherein the antibody or antigen binding fragment thereof
(i) binds to the extracellular domain of a human ACE2 protein, wherein binding of the human ACE2 protein by the antibody or antigen binding fragment thereof does not interfere with the enzymatic activity of the human ACE2 protein and does not induce internalization of the human ACE2 protein; and
(ii) binds to the same epitope on the extracellular domain of the human ACE2 protein as a reference antibody or antigen-binding fragment thereof, or competes for binding to the extracellular domain of the human ACE2 protein with the reference antibody or antigen-binding fragment thereof, wherein the reference antibody or antigen-binding fragment thereof comprises:
(a) a heavy chain variable domain selected from the group consisting of:
a heavy chain variable domain comprising a complementarity determining region (CDR)1-H comprising the amino acid sequence GFTFSSYA (SEQ ID NO: 43) or a variant thereof, a CDR2-H comprising the amino acid sequence ISGSGDRT (SEQ ID NO: 44) or a variant thereof, and a CDR3-H comprising the amino acid sequence AKDWAMVGADAFDI (SEQ ID NO: 45) or a variant thereof;
a heavy chain variable domain comprising a CDR1-H comprising the amino acid sequence GFTFSSYA (SEQ ID NO: 48) or a variant thereof; a CDR2-H comprising the amino acid sequence ISISGGST (SEQ ID NO: 49) or a variant thereof; and a CDR3-H comprising the amino acid sequence VKDWYIVGADAFDI (SEQ ID NO: 50) or a variant thereof;
a heavy chain variable domain comprising a CDR1-H comprising then amino acid sequence GFTFSSYA (SEQ ID NO: 53) or a variant thereof; a CDR2-H comprising the amino acid sequence INISGGST (SEQ ID NO: 54) or a variant thereof; and a CDR3-H comprising the amino acid sequence VKDWYIMGADAFDI (SEQ ID NO: 55) or a variant thereof;
a heavy chain variable domain comprising a CDR1-H comprising the amino acid sequence GFTFSNYA (SEQ ID NO:58) or a variant thereof; a CDR2-H comprising the amino acid sequence ISINGDRT (SEQ ID NO: 59) or a variant thereof; and a CDR3-H comprising the amino acid sequence AKDWAIVGADAFDV (SEQ ID NO: 60) or a variant thereof;
a heavy chain variable domain comprising a CDR1-H comprising the amino acid sequence GFTFNIYA (SEQ ID NO: 63) or a variant thereof; a CDR2-H comprising the amino acid sequence FSGSRYNT (SEQ ID NO: 64) or a variant thereof; and a CDR3-H comprising the amino acid sequence of AKEAVAGQFDY (SEQ ID NO: 65) or a variant thereof;
a heavy chain variable domain comprising a CDR1-H comprising the amino acid sequence DGSISGYY (SEQ ID NO: 68) or a variant thereof; a CDR2-H comprising the amino acid sequence IHYSGTT (SEQ ID NO: 69) or a variant thereof, and a CDR3-H comprising the amino acid sequence ARGPVFWYFDL (SEQ ID NO: 70) or a variant thereof;
a heavy chain variable domain comprising a CDR1-H comprising the amino acid sequence DGSISGYY (SEQ ID NO: 73) or a variant thereof; a CDR2-H comprising then amino acid sequence IYYSGTT (SEQ ID NO: 74) or a variant thereof, and a CDR3-H comprising the amino acid sequence ARGPVFWYFDL (SEQ ID NO: 75) or a variant thereof;
a heavy chain variable domain comprising a CDR1-H comprising the amino acid sequence GFTFSSYS (SEQ ID NO: 78) or a variant thereof, a CDR2-H comprising the amino acid sequence ITNGINNI (SEQ ID NO: 79) or a variant thereof; and a CDR3-H comprising the amino acid sequence ARYYYYYGMDV (SEQ ID NO: 80) or a variant thereof;
a heavy chain variable domain comprising a CDR1-H comprising the amino acid sequence GGSITSYY (SEQ ID NO: 83) or a variant thereof; a CDR2-H comprising the amino acid sequence IFSSGIT (SEQ ID NO: 84) or a variant thereof, and a CDR3-H comprising the amino acid sequence ARHDGFGWFDP (SEQ ID NO: 85) or a variant thereof;
a heavy chain variable domain comprising a CDR1-H comprising the amino acid sequence GFSLSTPGVA (SEQ ID NO: 88) or a variant thereof, a CDR2-H comprising the amino acid sequence IFWNDDE (SEQ ID NO: 89) or a variant thereof, and a CDR3-H comprising the amino acid sequence AHLALYWYFDF (SEQ ID NO: 90) or a variant thereof; and/or
(b) a light chain variable domain selected from the group consisting of:
a light chain variable domain comprising a CDR1-L comprising the amino acid sequence QGISNY (SEQ ID NO: 46) or a variant thereof; a CDR2-L comprising the amino acid sequence AAS or a variant thereof, and a CDR3-L comprising the amino acid sequence LQHSYYPYT (SEQ ID NO: 47) or a variant thereof;
a light chain variable domain comprising a CDR1-L comprising the amino acid sequence QGISNY (SEQ ID NO: 51) or a variant thereof; a CDR2-L comprising the amino acid sequence ATS or a variant thereof; and a CDR3-L comprising the amino acid sequence LQHSNYPYT (SEQ ID NO: 52) or a variant thereof;
a light chain variable domain comprising a CDR1-L comprising the amino acid sequence QGISNY (SEQ ID NO: 56) or a variant thereof; a CDR2-L comprising the amino acid sequence ATS or a variant thereof; and a CDR3-L comprising the amino acid sequence LQHSNYPYT (SEQ ID NO: 57) or a variant thereof;
a light chain variable domain comprising a CDR1-L comprising the amino acid sequence QVISNY (SEQ ID NO: 61) or a variant thereof; a CDR2-L comprising the amino acid sequence AGS or a variant thereof; and a CDR3-L comprising the amino acid sequence LQHNNYPYT (SEQ ID NO: 62) or a variant thereof;
a light chain variable domain comprising a CDR1-L comprising the amino acid sequence QSISSW (SEQ ID NO: 66) or a variant thereof; a CDR2-L comprising the amino acid sequence KAS or a variant thereof; and a CDR3-L comprising the amino acid sequence QQYNTYSRT (SEQ ID NO: 67) or a variant thereof;
a light chain variable domain comprising a CDR1-L comprising the amino acid sequence SGSINNNY (SEQ ID NO: 71) or a variant thereof; a CDR2-L comprising the amino acid sequence DDN or a variant thereof; and a CDR3-L comprising the amino acid sequence QSYDSSSRV (SEQ ID NO: 72) or a variant thereof;
a light chain variable domain comprising a CDR1-L comprising the amino acid sequence SGSISNSY (SEQ ID NO: 76) or a variant thereof; a CDR2-L comprising the amino acid sequence DDN or a variant thereof; and a CDR3-L comprising the amino acid sequence QSYDSSSRV (SEQ ID NO: 77) or a variant thereof;
a light chain variable domain comprising a CDR1-L comprising the amino acid sequence SLRNYY (SEQ ID NO: 81) or a variant thereof; a CDR2-L comprising the amino acid sequence GKN or a variant thereof; and a CDR3-L comprising the amino acid sequence NSRDSGGNHVV (SEQ ID NO: 82);
a light chain variable domain comprising a CDR1-L comprising the amino acid sequence QSVSSSY (SEQ ID NO: 86) or a variant thereof; a CDR2-L comprising the amino acid sequence GAS or a variant thereof; and a CDR3-L comprising the amino acid sequence QQYGSSPRT (SEQ ID NO: 87) or a variant thereof; and
a light chain variable domain comprising a CDR1-L comprising the amino acid sequence SSDVGSYNL (SEQ ID NO: 91) or a variant thereof, a CDR2-L comprising the amino acid sequence EGS or a variant thereof, and a CDR3-L comprising the amino acid sequence CSYAGSNIVV (SEQ ID NO: 92) or a variant thereof.

Another embodiment of the disclosure is directed to an isolated antibody or antigen binding fragment thereof, wherein the antibody or antigen binding fragment thereof comprises:
(a) a heavy chain variable domain selected from the group consisting of:
a heavy chain variable domain comprising a complementarity determining region (CDR)1-H comprising the amino acid sequence GFTFSSYA (SEQ ID NO: 43) or a variant thereof, a CDR2-H comprising the amino acid sequence ISGSGDRT (SEQ ID NO: 44) or a variant thereof, and a CDR3-H comprising the amino acid sequence AKDWAMVGADAFDI (SEQ ID NO: 45) or a variant thereof;
a heavy chain variable domain comprising a CDR1-H comprising the amino acid sequence GFTFSSYA (SEQ ID NO: 48) or a variant thereof; a CDR2-H comprising the amino acid sequence ISISGGST (SEQ ID NO: 49) or a variant thereof, and a CDR3-H comprising the amino acid sequence VKDWYIVGADAFDI (SEQ ID NO: 50) or a variant thereof;
a heavy chain variable domain comprising a CDR1-H comprising then amino acid sequence GFTFSSYA (SEQ ID NO: 53) or a variant thereof; a CDR2-H comprising the amino acid sequence INISGGST (SEQ ID NO: 54) or a variant thereof; and a CDR3-H comprising the amino acid sequence VKDWYIMGADAFDI (SEQ ID NO: 55) or a variant thereof;
a heavy chain variable domain comprising a CDR1-H comprising the amino acid sequence GFTFSNYA (SEQ ID NO: 58) or a variant thereof, a CDR2-H comprising the amino acid sequence ISINGDRT (SEQ ID NO: 59) or a variant thereof; and a CDR3-H comprising the amino acid sequence AKDWAIVGADAFDV (SEQ ID NO: 60) or a variant thereof;
a heavy chain variable domain comprising a CDR1-H comprising the amino acid sequence GFTFNIYA (SEQ ID NO: 63) or a variant thereof; a CDR2-H comprising the amino acid sequence FSGSRYNT (SEQ ID NO: 64) or a variant thereof, and a CDR3-H comprising the amino acid sequence of AKEAVAGQFDY (SEQ ID NO: 65) or a variant thereof;
a heavy chain variable domain comprising a CDR1-H comprising the amino acid sequence DGSISGYY (SEQ ID NO: 68) or a variant thereof; a CDR2-H comprising the amino acid sequence IHYSGTT (SEQ ID NO: 69) or a variant thereof; and a CDR3-H comprising the amino acid sequence ARGPVFWYFDL (SEQ ID NO: 70) or a variant thereof;
a heavy chain variable domain comprising a CDR1-H comprising the amino acid sequence DGSISGYY (SEQ ID NO: 73) or a variant thereof; a CDR2-H comprising then amino acid sequence IYYSGTT (SEQ ID NO: 74) or a variant thereof; and a CDR3-H comprising the amino acid sequence ARGPVFWYFDL (SEQ ID NO: 75) or a variant thereof;
a heavy chain variable domain comprising a CDR1-H comprising the amino acid sequence GFTFSSYS (SEQ ID NO: 78) or a variant thereof; a CDR2-H comprising the amino acid sequence ITNGINNI (SEQ ID NO: 79) or a variant thereof; and a CDR3-H comprising the amino acid sequence ARYYYYYGMDV (SEQ ID NO: 80) or a variant thereof;
a heavy chain variable domain comprising a CDR1-H comprising the amino acid sequence GGSITSYY (SEQ ID NO: 83) or a variant thereof; a CDR2-H comprising the amino acid sequence IFSSGIT (SEQ ID NO: 84) or a variant thereof; and a CDR3-H comprising the amino acid sequence ARHDGFGWFDP (SEQ ID NO: 85) or a variant thereof;
a heavy chain variable domain comprising a CDR1-H comprising the amino acid sequence GFSLSTPGVA (SEQ ID NO: 88) or a variant thereof; a CDR2-H comprising the amino acid sequence IFWNDDE (SEQ ID NO: 89) or a variant thereof; and a CDR3-H comprising the amino acid sequence AHLALYWYFDF (SEQ ID NO: 90) or a variant thereof; and/or
(b) a light chain variable domain selected from the group consisting of:
a light chain variable domain comprising a CDR1-L comprising the amino acid sequence QGISNY (SEQ ID NO: 46) or a variant thereof; a CDR2-L comprising the amino acid sequence AAS or a variant thereof; and a CDR3-L comprising the amino acid sequence LQHSYYPYT (SEQ ID NO: 47) or a variant thereof;
a light chain variable domain comprising a CDR1-L comprising the amino acid sequence QGISNY (SEQ ID NO: 51) or a variant thereof; a CDR2-L comprising the amino acid sequence ATS or a variant thereof; and a CDR3-L comprising the amino acid sequence LQHSNYPYT (SEQ ID NO: 52) or a variant thereof;
a light chain variable domain comprising a CDR1-L comprising the amino acid sequence QGISNY (SEQ ID NO: 56) or a variant thereof; a CDR2-L comprising the amino acid sequence ATS or a variant thereof; and a CDR3-L comprising the amino acid sequence LQHSNYPYT (SEQ ID NO: 57) or a variant thereof;
a light chain variable domain comprising a CDR1-L comprising the amino acid sequence QVISNY (SEQ ID NO: 61) or a variant thereof; a CDR2-L comprising the amino acid sequence AGS or a variant thereof, and a CDR3-L comprising the amino acid sequence LQHNNYPYT (SEQ ID NO: 62) or a variant thereof;
a light chain variable domain comprising a CDR1-L comprising the amino acid sequence QSISSW (SEQ ID NO: 66) or a variant thereof, a CDR2-L comprising the amino acid sequence KAS or a variant thereof, and a CDR3-L comprising the amino acid sequence QQYNTYSRT (SEQ ID NO: 67) or a variant thereof;
a light chain variable domain comprising a CDR1-L comprising the amino acid sequence SGSINNNY (SEQ ID NO: 71) or a variant thereof, a CDR2-L comprising the amino acid sequence DDN or a variant thereof; and a CDR3-L comprising the amino acid sequence QSYDSSSRV (SEQ ID NO: 72) or a variant thereof;
a light chain variable domain comprising a CDR1-L comprising the amino acid sequence SGSISNSY (SEQ ID NO: 76) or a variant thereof, a CDR2-L comprising the amino acid sequence DDN or a variant thereof, and a CDR3-L comprising the amino acid sequence QSYDSSSRV (SEQ ID NO: 77) or a variant thereof;
a light chain variable domain comprising a CDR1-L comprising the amino acid sequence SLRNYY (SEQ ID NO: 81) or a variant thereof, a CDR2-L comprising the amino acid sequence GKN or a variant thereof, and a CDR3-L comprising the amino acid sequence NSRDSGGNHVV (SEQ ID NO: 82) or a variant thereof;
a light chain variable domain comprising a CDR1-L comprising the amino acid sequence QSVSSSY (SEQ ID NO: 86) or a variant thereof, a CDR2-L comprising the amino acid sequence GAS or a variant thereof, and a CDR3-L comprising the amino acid sequence QQYGSSPRT (SEQ ID NO: 87) or a variant thereof; and
a light chain variable domain comprising a CDR1-L comprising the amino acid sequence SSDVGSYNL (SEQ ID NO: 91) or a variant thereof, a CDR2-L comprising the amino acid sequence EGS or a variant thereof, and a CDR3-L comprising the amino acid sequence CSYAGSNIVV (SEQ ID NO: 92) or a variant thereof.

In one embodiment, the disclosure is directed to an isolated antibody or antigen binding fragment thereof, wherein the antibody or antigen binding fragment thereof comprises:
(a) a heavy chain variable domain comprising an amino acid sequence having at least 90% sequence identity to the amino acid sequence as set forth in SEQ ID NOs: 6, 10, 14, 18, 22, 26, 30, 34, 38, or 42; and/or
(b) a light chain variable domain comprising an amino acid sequence having at least 90% sequence identity to the amino acid sequence as set forth in SEQ ID NOs: 4, 8, 12, 16, 20, 24, 28, 32, 36, or 40.

In another embodiment, the disclosure is directed to an isolated antibody or antigen binding fragment thereof, wherein the antibody or antigen binding fragment thereof comprises: a heavy chain variable domain comprising
a CDR1-H comprising the amino acid sequence GFTFSSYA (SEQ ID NO: 43) or a variant thereof, a CDR2-H comprising the amino acid sequence ISGSGDRT (SEQ ID NO: 44) or a variant thereof, and a CDR3-H comprising the amino acid sequence AKDWAMVGADAFDI (SEQ ID NO: 45) or a variant thereof; and/or
a CDR1-H comprising the amino acid sequence GFTFSSYA (SEQ ID NO: 48) or a variant thereof; a CDR2-H comprising the amino acid sequence ISISGGST (SEQ ID NO: 49) or a variant thereof, and a CDR3-H comprising the amino acid sequence VKDWYIVGADAFDI (SEQ ID NO: 50) or a variant thereof; and/or
a CDR1-H comprising then amino acid sequence GFTFSSYA (SEQ ID NO: 53) or a variant thereof; a CDR2-H comprising the amino acid sequence INISGGST (SEQ ID NO: 54) or a variant thereof, and a CDR3-H comprising the amino acid sequence VKDWYIMGADAFDI (SEQ ID NO: 55) or a variant thereof; and/or
a CDR1-H comprising the amino acid sequence GFTFSNYA (SEQ ID NO: 58) or a variant thereof, a CDR2-H comprising the amino acid sequence ISINGDRT (SEQ ID NO: 59) or a variant thereof, and a CDR3-H comprising the amino acid sequence AKDWAIVGADAFDV (SEQ ID NO: 60) or a variant thereof; and/or
a CDR1-H comprising the amino acid sequence GFTFNIYA (SEQ ID NO: 63) or a variant thereof; a CDR2-H comprising the amino acid sequence FSGSRYNT (SEQ ID NO: 64) or a variant thereof; and a CDR3-H comprising the amino acid sequence of AKEAVAGQFDY (SEQ ID NO: 65) or a variant thereof; and/or
a CDR1-H comprising the amino acid sequence DGSISGYY (SEQ ID NO: 68) or a variant thereof; a CDR2-H comprising the amino acid sequence IHYSGTT (SEQ ID NO: 69) or a variant thereof; and a CDR3-H comprising the amino acid sequence ARGPVFWYFDL (SEQ ID NO: 70) or a variant thereof; and/or
a CDR1-H comprising the amino acid sequence DGSISGYY (SEQ ID NO: 73) or a variant thereof; a CDR2-H comprising then amino acid sequence IYYSGTT (SEQ ID NO: 74) or a variant thereof; and a CDR3-H comprising the amino acid sequence ARGPVFWYFDL (SEQ ID NO: 75) or a variant thereof; and/or
a CDR1-H comprising the amino acid sequence GFTFSSYS (SEQ ID NO: 78) or a variant thereof; a CDR2-H comprising the amino acid sequence ITNGINNI (SEQ ID NO: 86) or a variant thereof; and a CDR3-H comprising the amino acid sequence ARYYYYYGMDV (SEQ ID NO: 80) or a variant thereof; and/or
a CDR1-H comprising the amino acid sequence GGSITSYY (SEQ ID NO: 83) or a variant thereof; a CDR2-H comprising the amino acid sequence IFSSGIT (SEQ ID NO: 84) or a variant thereof; and a CDR3-H comprising the amino acid sequence ARHDGFGWFDP (SEQ ID NO: 85) or a variant thereof; and/or
a CDR1-H comprising the amino acid sequence GFSLSTPGVA (SEQ ID NO: 88) or a variant thereof; a CDR2-H comprising the amino acid sequence IFWNDDE (SEQ ID NO: 89) or a variant thereof; and a CDR3-H comprising the amino acid sequence AHLALYWYFDF (SEQ ID NO: 90) or a variant thereof; and/or
a light chain variable domain comprising: a CDR1-L comprising the amino acid sequence QGISNY (SEQ ID NO: 46) or a variant thereof; a CDR2-L comprising the amino acid sequence AAS or a variant thereof; and a CDR3-L comprising the amino acid sequence LQHSYYPYT (SEQ ID NO: 47) or a variant thereof; and/or
a CDR1-L comprising the amino acid sequence QGISNY (SEQ ID NO: 51) or a variant thereof; a CDR2-L comprising the amino acid sequence ATS or a variant thereof; and a CDR3-L comprising the amino acid sequence LQHSNYPYT (SEQ ID NO: 52) or a variant thereof; and/or
a CDR1-L comprising the amino acid sequence QGISNY (SEQ ID NO: 56) or a variant thereof, a CDR2-L comprising the amino acid sequence ATS or a variant thereof; and a CDR3-L comprising the amino acid sequence LQHSNYPYT (SEQ ID NO: 57) or a variant thereof; and/or
a CDR1-L comprising the amino acid sequence QVISNY (SEQ ID NO: 61) or a variant thereof, a CDR2-L comprising the amino acid sequence AGS or a variant thereof; and a CDR3-L comprising the amino acid sequence LQHNNYPYT (SEQ ID NO: 62) or a variant thereof; and/or
a CDR1-L comprising the amino acid sequence QSISSW (SEQ ID NO: 66) or a variant thereof, a CDR2-L comprising the amino acid sequence KAS or a variant thereof, and a CDR3-L comprising the amino acid sequence QQYNTYSRT (SEQ ID NO: 67) or a variant thereof; and/or
a CDR1-L comprising the amino acid sequence SGSINNNY (SEQ ID NO: 71) or a variant thereof; a CDR2-L comprising the amino acid sequence DDN or a variant thereof; and a CDR3-L comprising the amino acid sequence QSYDSSSRV (SEQ ID NO: 72) or a variant thereof; and/or
a CDR1-L comprising the amino acid sequence SGSISNSY (SEQ ID NO: 76) or a variant thereof; a CDR2-L comprising the amino acid sequence DDN or a variant thereof, and a CDR3-L comprising the amino acid sequence QSYDSSSRV (SEQ ID NO: 77) or a variant thereof; and/or
a CDR1-L comprising the amino acid sequence SLRNYY (SEQ ID NO: 81) or a variant thereof; a CDR2-L comprising the amino acid sequence GKN or a variant thereof; and a CDR3-L comprising the amino acid sequence NSRDSGGNHVV (SEQ ID NO: 82); and/or
a CDR1-L comprising the amino acid sequence QSVSSSY (SEQ ID NO: 86) or a variant thereof, a CDR2-L comprising the amino acid sequence GAS or a variant thereof, and a CDR3-L comprising the amino acid sequence QQYGSSPRT (SEQ ID NO: 87) or a variant thereof; and/or
a CDR1-L comprising the amino acid sequence SSDVGSYNL (SEQ ID NO: 91) or a variant thereof; a CDR2-L comprising the amino acid sequence EGS or a variant thereof; and a CDR3-L comprising the amino acid sequence CSYAGSNIVV (SEQ ID NO: 92) or a variant thereof.

Another embodiment of the disclosure is directed to an isolated antibody or antigen-binding fragment, comprising:
(a) a heavy chain variable domain comprising a complementarity determining region (CDR)1-H comprising the amino acid sequence GFTFSSYA (SEQ ID NO: 43) or a variant thereof, a CDR2-H comprising the amino acid sequence ISGSGDRT (SEQ ID NO: 44) or a variant thereof, and a CDR3-H comprising the amino acid sequence AKDWAMVGADAFDI (SEQ ID NO: 45) or a variant thereof, and a light chain variable domain comprising a CDR1-L comprising the amino acid sequence QGISNY (SEQ ID NO: 46) or a variant thereof; a CDR2-L comprising the amino acid sequence AAS or a variant thereof, and a CDR3-L comprising the amino acid sequence LQHSYYPYT (SEQ ID NO: 47) or a variant thereof;
(b) a heavy chain variable domain comprising a CDR1-H comprising the amino acid sequence GFTFSSYA (SEQ ID NO: 48) or a variant thereof, a CDR2-H comprising the amino acid sequence ISISGGST (SEQ ID NO: 49) or a variant thereof, and a CDR3-H comprising the amino acid sequence VKDWYIVGADAFDI (SEQ ID NO: 50) or a variant thereof, and a light chain variable domain comprising a CDR1-L comprising the amino acid sequence QGISNY (SEQ ID NO: 51) or a variant thereof; a CDR2-L comprising the amino acid sequence ATS or a variant thereof, and a CDR3-L comprising the amino acid sequence LQHSNYPYT (SEQ ID NO: 52) or a variant thereof;
(c) a heavy chain variable domain comprising a CDR1-H comprising then amino acid sequence GFTFSSYA (SEQ ID NO: 53) or a variant thereof, a CDR2-H comprising the amino acid sequence INISGGST (SEQ ID NO: 54) or a variant thereof, and a CDR3-H comprising the amino acid sequence VKDWYIMGADAFDI (SEQ ID NO: 55) or a variant thereof, and a light chain variable domain comprising a CDR1-L comprising the amino acid sequence QGISNY (SEQ ID NO: 56) or a variant thereof, a CDR2-L comprising the amino acid sequence ATS or a variant thereof, and a CDR3-L comprising the amino acid sequence LQHSNYPYT (SEQ ID NO: 57) or a variant thereof;
(d) a heavy chain variable domain comprising a CDR1-H comprising the amino acid sequence GFTFSNYA (SEQ ID NO: 58) or a variant thereof, a CDR2-H comprising the amino acid sequence ISINGDRT (SEQ ID NO: 59) or a variant thereof; and a CDR3-H comprising the amino acid sequence AKDWAIVGADAFDV (SEQ ID NO: 60) or a variant thereof; and a light chain variable domain comprising a CDR1-L comprising the amino acid sequence QVISNY (SEQ ID NO: 61) or a variant thereof; a CDR2-L comprising the amino acid sequence AGS or a variant thereof; and a CDR3-L comprising the amino acid sequence LQHNNYPYT (SEQ ID NO: 62) or a variant thereof;
(e) a heavy chain variable domain comprising a CDR1-H comprising the amino acid sequence GFTFNIYA (SEQ ID NO: 63) or a variant thereof; a CDR2-H comprising the amino acid sequence FSGSRYNT (SEQ ID NO: 64) or a variant thereof; and a CDR3-H comprising the amino acid sequence of AKEAVAGQFDY (SEQ ID NO: 65) or a variant thereof; and a light chain variable domain comprising a CDR1-L comprising the amino acid sequence QSISSW (SEQ ID NO: 66) or a variant thereof; a CDR2-L comprising the amino acid sequence KAS or a variant thereof; and a CDR3-L comprising the amino acid sequence QQYNTYSRT (SEQ ID NO: 67) or a variant thereof;
(f) heavy chain variable domain comprising a CDR1-H comprising the amino acid sequence DGSISGYY (SEQ ID NO: 68) or a variant thereof; a CDR2-H comprising the amino acid sequence IHYSGTT (SEQ ID NO: 69) or a variant thereof; and a CDR3-H comprising the amino acid sequence ARGPVFWYFDL (SEQ ID NO: 70) or a variant thereof; and a light chain variable domain comprising a CDR1-L comprising the amino acid sequence SGSINNNY (SEQ ID NO: 71) or a variant thereof; a CDR2-L comprising the amino acid sequence DDN or a variant thereof; and a CDR3-L comprising the amino acid sequence QSYDSSSRV (SEQ ID NO: 72) or a variant thereof;
(g) a heavy chain variable domain comprising a CDR1-H comprising the amino acid sequence DGSISGYY (SEQ ID NO: 73) or a variant thereof; a CDR2-H comprising then amino acid sequence IYYSGTT (SEQ ID NO: 74) or a variant thereof; and a CDR3-H comprising the amino acid sequence ARGPVFWYFDL (SEQ ID NO: 75) or a variant thereof; and a light chain variable domain comprising a CDR1-L comprising the amino acid sequence SGSISNSY (SEQ ID NO: 76) or a variant thereof; a CDR2-L comprising the amino acid sequence DDN or a variant thereof; and a CDR3-L comprising the amino acid sequence QSYDSSSRV (SEQ ID NO: 77) or a variant thereof;
(h) a heavy chain variable domain comprising a CDR1-H comprising the amino acid sequence GFTFSSYS (SEQ ID NO: 78) or a variant thereof, a CDR2-H comprising the amino acid sequence ITNGINNI (SEQ ID NO: 79) or a variant thereof, and a CDR3-H comprising the amino acid sequence ARYYYYYGMDV (SEQ ID NO: 80) or a variant thereof, and a light chain variable domain comprising a CDR1-L comprising the amino acid sequence SLRNYY (SEQ ID NO: 81) or a variant thereof, a CDR2-L comprising the amino acid sequence GKN or a variant thereof, and a CDR3-L comprising the amino acid sequence NSRDSGGNHVV (SEQ ID NO: 82);
(i) a heavy chain variable domain comprising a CDR1-H comprising the amino acid sequence GGSITSYY (SEQ ID NO: 83) or a variant thereof, a CDR2-H comprising the amino acid sequence IFSSGIT (SEQ ID NO: 84) or a variant thereof, and a CDR3-H comprising the amino acid sequence ARHDGFGWFDP (SEQ ID NO: 85) or a variant thereof, and a light chain variable domain comprising a CDR1-L comprising the amino acid sequence QSVSSSY (SEQ ID NO: 86) or a variant thereof, a CDR2-L comprising the amino acid sequence GAS or a variant thereof, and a CDR3-L comprising the amino acid sequence QQYGSSPRT (SEQ ID NO: 87) or a variant thereof; and/or
(j) a heavy chain variable domain comprising a CDR1-H comprising the amino acid sequence GFSLSTPGVA (SEQ ID NO: 88) or a variant thereof, a CDR2-H comprising the amino acid sequence IFWNDDE (SEQ ID NO: 89) or a variant thereof, and a CDR3-H comprising the amino acid sequence AHLALYWYFDF (SEQ ID NO: 90) or a variant thereof;
and a light chain variable domain comprising a CDR1-L comprising the amino acid sequence SSDVGSYNL (SEQ ID NO: 91) or a variant thereof, a CDR2-L comprising the amino acid sequence EGS or a variant thereof, and a CDR3-L comprising the amino acid sequence CSYAGSNIVV (SEQ ID NO: 92) or a variant thereof.

Another embodiment of the disclosure is directed to an isolated antibody or antigen-binding fragment thereof, comprising: a heavy chain variable domain comprising the amino acid sequence as set forth in SEQ ID NO: 6, 10, 14, 18, 22, 26, 30, 34, 38, or 42; and/or a light chain variable domain comprising the amino acid sequence as set forth in SEQ ID NO: 4, 8, 12, 16, 20, 24, 28, 32, 36, or 40.

Another embodiment of the current disclosure is directed to an isolated antibody or antigen-binding fragment thereof, comprising:
(a) a heavy chain variable domain comprising an amino acid sequence having at least 90% identity to the amino acid sequence as set forth in SEQ ID NO: 6, and a light chain variable domain comprising an amino acid sequence having at least 90% identity to the amino acid sequence as set forth in SEQ ID NO: 4;
(b) a heavy chain variable domain comprising an amino acid sequence having at least 90% identity to the amino acid sequence as set forth in SEQ ID NO: 10, and a light chain variable domain comprising an amino acid sequence having at least 90% identity to the amino acid sequence as set forth in SEQ ID NO: 8;
(c) a heavy chain variable domain comprising an amino acid sequence having at least 90% identity to the amino acid sequence as set forth in SEQ ID NO: 14, and a light chain variable domain comprising an amino acid sequence having at least 90% identity to the amino acid sequence as set forth in SEQ ID NO: 12;
(d) a heavy chain variable domain comprising an amino acid sequence having at least 90% identity to the amino acid sequence as set forth in SEQ ID NO: 18 and a light chain variable domain comprising an amino acid sequence having at least 90% identity to the amino acid sequence as set forth in SEQ ID NO: 16;
(e) a heavy chain variable domain comprising an amino acid sequence having at least 90% identity to the amino acid sequence as set forth in SEQ ID NO: 22, and a light chain variable domain comprising an amino acid sequence having at least 90% identity to the amino acid sequence as set forth in SEQ ID NO: 20;
(f) a heavy chain variable domain comprising an amino acid sequence having at least 90% identity to the amino acid sequence as set forth in SEQ ID NO: 26, and a light chain variable domain comprising an amino acid sequence having at least 90% identity to the amino acid sequence as set forth in SEQ ID NO: 24;
(g) a heavy chain variable domain comprising an amino acid sequence having at least 90% identity to the amino acid sequence as set forth in SEQ ID NO: 30, and a light chain variable domain comprising an amino acid sequence having at least 90% identity to the amino acid sequence as set forth in SEQ ID NO: 28;
(h) a heavy chain variable domain comprising an amino acid sequence having at least 90% identity to the amino acid sequence as set forth in SEQ ID NO: 34, and a light chain variable domain comprising an amino acid sequence having at least 90% identity to the amino acid sequence as set forth in SEQ ID NO: 32;
(i) a heavy chain variable domain comprising an amino acid sequence having at least 90% identity to the amino acid sequence as set forth in SEQ ID NO: 38, and a light chain variable domain comprising an amino acid sequence having at least 90% identity to the amino acid sequence as set forth in SEQ ID NO: 36; and/or
(j) a heavy chain variable domain comprising an amino acid sequence having at least 90% identity to the amino acid sequence as set forth in SEQ ID NO: 42, and a light chain variable domain comprising an amino acid sequence having at least 90% identity to the amino acid sequence as set forth in SEQ ID NO: 40.

In one embodiment, the current disclosure is directed to an antibody or antigen-binding fragment thereof, wherein the heavy chain immunoglobulin variable domain is linked to an IgG, IgG1 or IgG4 heavy chain constant region and the light chain immunoglobulin variable region is linked to a human kappa or lambda light chain constant region. In one embodiment, the heavy chain immunoglobulin variable domain is linked to a human IgG1 constant region comprising substitutions at L234 L235 (LALA) that abolish FcR-gamma interaction, and/or substitutions at M428 N434 (LS) that enhance interaction with the neonatal Fc receptor to prolong antibody half-life in human.

In one embodiment, the antibody or antigen-binding fragment is a multispecific (e.g., bispecific) antibody or antigen-binding fragment.

In some embodiments of the disclosure, the antibody or antigen-binding fragment thereof comprises one or more of the following additional properties: (a) binds to the human ACE2 protein with a K_{D} of about 10⁻⁹ M, 10⁻¹⁰M, 10⁻¹¹ M, or 10⁻¹² M; e.g., about 7.66 nM; (b) blocks binding of a coronavirus to the human ACE2 protein; (c) inhibits infection by a coronavirus.

In some embodiments of the disclosure, the coronavirus is a SARS-CoV, a SARS-CoV-2, a Pangolin CoV, a Bat CoV, any other member of the betacoronavirus genus or sarbecovirus sub-genus that uses ACE2 to enter cells or any member of the alphacoronavirus genus, such as human coronavirus HCoV-NL-63 that uses ACE2 to enter cells. In some embodiments, the SARS-CoV-2 is a Wuhan-hu-1 variant, an Alpha variant, a Beta variant, a Delta variant, an Omicron variant, a derivative thereof, or a combination thereof such, as BA.2, BA.4, BA.5.

In some embodiments of the disclosure, the anti-ACE2 antibodies or antigen binding fragments thereof compete for binding to the same epitope as the coronavirus spike proteins. In some embodiments, the antibody or antigen binding fragment thereof binds to the same epitope on the extracellular domain of the human ACE2 protein as does the receptor binding domain (RBD) of the SARS-CoV-2 spike protein, or the RBD of any other member of the betacoronavirus genus or sarbecovirus sub-genus that uses ACE2 to enter cells or any member of the alphacoronavirus genus which uses ACE2 to enter cells. In some embodiments, the member of the alphacoronavirus genus is human coronavirus HCoV-NL-63.

In another aspect, the disclosure is directed to an isolated nucleic acid encoding an immunoglobulin chain or variable region thereof of the antibody according to the disclosure. One aspect of the disclosure is a vector comprising the isolated nucleic acid encoding an immunoglobulin chain or variable region thereof of the antibody according to the disclosure. Another aspect of the disclosure is a host cell comprising the isolated nucleic acid encoding an immunoglobulin chain or variable region thereof of the antibody according to the disclosure or the vector comprising the isolated nucleic acid encoding an immunoglobulin chain or variable region thereof of the antibody according to the disclosure. In one embodiment, the host cell is an Expi293 cell.

Another aspect of the disclosure is a method for making an antibody or antigen-binding fragment thereof according to the disclosure, or an immunoglobulin chain thereof. In one embodiment, the method comprises: (a) introducing one or more nucleic acids encoding an immunoglobulin chain of antibody or antigen-binding fragment thereof into a host cell; (b) culturing the host cell in a medium to express the immunoglobulin chain(s); and (c) optionally, isolating the immunoglobulin chain or antibody or antigen-binding fragment thereof from the host cell and/or the medium. In some embodiments, the host cell is an Expi293 cell. In some embodiments, the host cell is a CHO cell.

Another aspect of the current disclosure is a pharmaceutical composition comprising an antibody or antigen-binding fragment according to the disclosure and a pharmaceutically acceptable carrier.

Another aspect of the current disclosure is a method for treating or preventing infection caused by coronavirus in a subject in need thereof, comprising administering to the subject a therapeutically effective amount of an antibody or antigen-binding fragment thereof as described in the disclosure. In one embodiment, the coronavirus is a SARS-CoV, a SARS-CoV-2, a Pangolin CoV, a Bat CoV, any other member of the betacoronavirus genus or sarbecovirus sub-genus that uses ACE2 to enter cells or any member of the alphacoronavirus genus which uses ACE2 to enter cells. In some embodiments, the member of the alphacoronavirus genus is human coronavirus HCoV-NL-63. In some embodiments, the SARS-CoV-2 is a Wuhan-hu-1 variant, an Alpha variant, a Beta variant, a Delta variant, an Omicron variant, or a combination or derivative thereof, such as BA.2, BA.4, BA.5. In some embodiments, the antibody or antigen-binding fragment thereof is injected into the subject subcutaneously, intravenously, or intramuscularly.

### BRIEF DESCRIPTION OF THE DRAWINGS

This patent or application contains at least one drawing executed in color. Copies of this patent or patent application publication with color drawing(s) will be provided by the Office upon request and payment of the necessary fee.

FIG. 1A-J. Inhibition of pseudovirus infection by anti-human ACE2 antibodies shown through inhibition curves for anti-hACE2 antibody inhibition of pseudotyped virus infection determined using HIV-1 based pseudoviruses and Huh7 target cells where the 05B04 LC/05D06 HC antibody is a hybrid antibody generated by coexpression of the 05B04 light chain with the heavy chain of a clonally related antibody, 05D06.

FIG. 2. Inhibition of authentic SARS-CoV-2 infection by anti-human ACE2 antibodies shown through inhibition curves for anti-hACE2 antibody (05B04) inhibition of authentic SARS-CoV-2 infection determined using SARS-CoV-2, USA_WA/2020, and Vero E6 or Huh7 target cells.

FIG. 3. Representation of specific binding to hACE2 by human anti-ACE2 antibodies through flow cytometric analysis using human anti-hACE2 antibodies. Unmodified A549 cells (upper row) or modified derivatives engineered to express hACE2 (lower row) were incubated in the presence (solid lines) or absence (dotted lines) of the indicated human ACE2 antibodies.

FIG. 4A-D. Shows binding to hACE2 by human anti-ACE2 antibodies. Anti-hACE2 antibodies were immobilized on a Protein G sensor chip (cytiva) at the indicated concentrations and the binding of 8×His tagged ACE2 extracellular domain measured. A. shows anti-hACE2 antibody 2G7A1 with a KD of 3.24e-9. B shows anti-hACE2 antibody 05B04 with a KD of 2.76e-10. C shows anti-hACE2 antibody 05H02 with a KD of 1.80e-9. D shows anti-hACE2 antibody 05B04/05D06 with a KD of 2.71e-9.

FIG. 5. Depiction of ACE 2 activity measured with an ACE2 Inhibitor Screening Assay Kit with a fluorogenic substrate and fluorescence intensity measured. 10 ng of ACE2/well was used and fluorescence intensity (excitation 555 nm and emission 585 nm) measured. Antibodies added at 0.08 to 50 μg/ml (up to 1000×IC₅₀ for antiviral activity). MLN-4760 used as positive control ACE2 inhibitor with a reported IC₅₀ of 0.03 M.

FIG. 6A-E. Representation that anti-human ACE2 antibodies do not induce ACE2 internalization. Live A549 cells expressing a C-terminally (intracellular) HA-tagged human ACE2 receptor were incubated with the indicated anti-human ACE2 antibodies for 1 hr at 37° C. Cells were then fixed and permeabilized. Total human HA-ACE2 was then labelled with a mouse anti-HA-tag antibody. The internalization of the HA-ACE2 protein and human anti-ACE2 antibodies was evaluated by staining with goat anti-human Alexafluor-488 (green, left) or goat anti-mouse Alexafluor 594 (red, center) or both (right) antibodies. Blue stain (DAPI) indicates cell nuclei.

FIG. 7. Shows human ACE2 antibody levels following injection in human ACE2 knock-in mice. Plasma antibody concentrations in mice following subcutaneous injection of 250 g of 05B04 antibody (approximately equivalent to 12.5 mg/kg) into each of 5 hACE2 knock-in mice PK on day 0. Dashed line=IC₅₀ for inhibition of SARS-CoV-2 pseudovirus infection.

FIG. 8. Protection of hACE2 knock-in mice from SARS-CoV-2 infection by anti-hACE2 antibodies. Human ACE2 knock-in mice B6.12952(Cg)-Ace2tml(ACE2)Dwnt/J (five mice per treatment group) were injected with 250 ug (equivalent to ˜12.5 mg/kg) of the 05B04 or 05H02 anti-human ACE2 antibodies. At 2 days after antibody injection mice were challenged intranasally with SARS-CoV-2, USA_WA/2020 P3, 1×10⁵ PFU/mouse (virus titers measured on VeroE6 cells). At 3 days after infection, mouse lungs were harvested, and RNA extracted. The number of viral genomes per microgram of total lung RNA was measured by qRT-PCR and the standard is 2019-nCoV_N_Positive Control 10006625 (Integrated DNA technologies).

### DETAILED DESCRIPTION

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value).

### General Terminology

The term “isolated” as used herein in reference to an antibody or an antigen-binding fragment thereof refers to an antibody or an antigen binding fragment thereof that: (1) is not associated with naturally associated components that accompany it in its native state; (2) is free of other proteins from the same species; (3) is expressed by a cell from a different species; and/or (4) does not occur in nature.

The term “antibody” refers to an immunoglobulin molecule. The general structure of antibodies in vertebrates now is well understood. See Edelman, G. M., Ann. NY Acad Sci., 190:5 (1971). Antibodies consist of two light polypeptide chains of molecular weight approximately 23,000 Daltons (the “light chain”), and two heavy chains of molecular weight 53,000-70,000 Daltons (the “heavy chain”). The four chains are joined by disulfide bonds in a “Y” configuration wherein the light chains bracket the heavy chains starting at the mouth of the “Y” configuration. The “branch” portion of the “Y” configuration is designated the Fab region; the stem portion of the “Y” configuration is designated the Fe region. The amino acid sequence orientation runs from the N-terminal end at the top of the “Y” configuration to the C-terminal end at the bottom of each chain. The N-terminal end possesses the variable region having specificity for the antigen that elicited it, and is approximately 100 amino acids in length, there being slight variations between light and heavy chain and from antibody to antibody. The variable region is linked in each chain to a constant region that extends the remaining length of the chain and that within a particular class of antibody does not vary with the specificity of the antibody (i.e., the antigen eliciting it). There are five known major classes of constant regions that determine the class of the immunoglobulin molecule (IgG, IgM, IgA, IgD, and IgE corresponding to γ, μ, α, δ, and ε (gamma, mu, alpha, delta, and epsilon, respectfully) heavy chain constant regions). The constant region or class determines subsequent effector function of the antibody, including activation of complement (see Kabat, E. A, Structural Concepts in Immunology and Immunochemistry, 2nd Ed., p. 413-436, New York, NY: Holt, Rinehart, Winston (1976)), and other cellular responses (see Andrews et al., Clinical Immunology, pp. 1-18, W. B. Sanders, Philadelphia, PA (1980); Kohl et al., Immunology, 48: 187 (1983)); while the variable region determines the antigen with which it will react. Light chains are classified as either κ (kappa) or λ (lambda). Each heavy chain class can be prepared with either kappa or lambda light chain. The light and heavy chains are covalently bonded to each other, and the “tail” portions of the two heavy chains are bonded to each other by covalent disulfide linkages when the immunoglobulins are generated either by hybridomas or by B-cells.

The term “antibody” as used herein encompasses murine, humanized, human and chimeric antibodies, and antibodies in a multimeric form, such as dimers, trimers, or higher-order multimers of monomeric antibodies. The term “antibody” as used herein encompasses monospecific and multispecific (e.g., bispecific) antibodies, and any other modified configuration of the immunoglobulin molecule that comprises an antigen recognition site of the required specificity. Further, the term “antibody” is not limited by any particular method of producing the antibody. For example, it includes monoclonal antibodies, recombinant antibodies, and polyclonal antibodies. The term “antibody” includes antibodies of all classes and subclasses, e.g., an IgG, IgA, IgD, IgE or IgM antibody, such as IgG1, IgG2, IgG3 or IgG4 antibody.

The term “antigen-binding fragment” of an antibody refers to one or more portions of a full-length antibody that are responsible for and involved in binding to the antigen. Examples of antigen-binding fragments include Fab fragments, F(ab′)₂ fragments, Fd fragments, Fv fragments, single chain Fv (scFv) molecules, a variable domain (VH or VL), a molecule comprising one or more VH and/or VL. An antigen-binding fragment can be synthetic, recombinantly produced, or enzymatically derived.

Typically, the variable domains of both the heavy and light immunoglobulin chains comprise three hypervariable regions, also called complementarity determining regions (CDRs), located within relatively conserved framework regions (FR). In general, from N-terminal to C-terminal, both light and heavy chains variable domains comprise FR1, CDR1, FR2, CDR2, FR3, CDR3 and FR4. In an embodiment of the disclosure, the assignment of amino acids to each domain is in accordance with the definitions of Sequences of Proteins of Immunological Interest, Kabat, et al., National Institutes of Health, Bethesda, Md.; 5th ed.; NIH Publ. No. 91-3242 (1991); Kabat, Adv. Prot. Chem. 32:1-75(1978); Kabat, et al., J. Biol. Chem. 252:6609-6616 (1977); Chothia, et al., J Mol. Biol. 196:901-917 (1987) or Chothia, et al., Nature 342:878-883 (1989).

The expression “variable region” or “VR” refers to the domains within each pair of light and heavy chains in an antibody that are involved directly in binding of the antibody to the antigen. Each heavy chain has at one end a variable region (VH) followed by a number of constant domains. Each light chain has a variable region (VL) at one end and a constant domain at its other end; the constant domain of the light chain is aligned with the first constant domain of the heavy chain, and the light chain variable domain is aligned with the variable domain of the heavy chain.

The expressions “complementarity-determining region,” “hypervariable region,” and “CDR” refer to one or more of the hyper-variable or complementarity-determining regions (“CDRs”) found in the variable regions of light or heavy chains of an antibody (See Kabat et al., Sequences of Proteins of Immunological Interest, 4th ed., Bethesda, MD: U.S. Dept. of Health and Human Services, Public Health Service, National Institutes of Health (1987)). These expressions include the hypervariable regions as defined by Kabat et al. (Sequences of Proteins of Immunological Interest, NIH Publication No. 91-3242, Bethesda, MD: U.S. Dept. of Health and Human Services, National Institutes of Health (1983)) or the hypervariable loops in 3-dimensional structures of antibodies (Chothia and Lesk, J Mal. Biol., 196:901-917 (1987)). The CDRs in each chain are held in close proximity by framework regions (“FRs”) and, with the CDRs from the other chain, contribute to the formation of the antigen binding site. Within the CDRs there are select amino acids that have been described as the selectivity determining regions (“SDRs”) that represent the critical contact residues used by the CDR in the antibody-antigen interaction (see Kashmiri et al., Methods, 36(1):25-34 (2005)).

The term “human antibody” refers to an antibody consisting of amino acid sequences of human immunoglobulin sequences only. A human antibody may contain murine carbohydrate chains if produced in a mouse, in a mouse cell or in a hybridoma derived from a mouse cell. Human antibodies may be prepared in a variety of ways known in the art.

The term “humanized antibody” includes an antibody that contains some or all of the CDRs from a non-human animal antibody while the framework and constant regions of the antibody contain amino acid residues derived from human antibody sequences. Humanized antibodies can be produced by grafting CDRs from a mouse antibody into human framework sequences, and in some instances followed by back substitution of certain human framework residues for the corresponding mouse residues from the source antibody. The term “humanized antibody” also includes an antibody of non-human origin in which, typically in one or more variable regions, one or more epitopes have been removed, that have a high propensity of constituting a human T-cell and/or B-cell epitope, for purposes of reducing immunogenicity. The amino acid sequence of the epitope can be removed in full or in part. However, typically the amino acid sequence is altered by substituting one or more of the amino acids constituting the epitope for one or more other amino acids, thereby changing the amino acid sequence into a sequence that does not constitute a human T-cell and/or B-cell epitope. The amino acids are substituted by amino acids that are present at the corresponding position(s) in a corresponding human variable heavy or variable light chain as the case may be.

The term “chimeric antibody” refers to an antibody that comprises amino acid sequences derived from two different species such as human and mouse, typically a combination of mouse variable (from heavy and light chains) regions and human constant (heavy and light chains) regions.

A “single-chain antibody” (scFv) consists of a single polypeptide chain comprising a VL domain linked to a VH domain wherein VL domain and VH domain are paired to form a monovalent molecule. Single chain antibody can be prepared according to method known in the art (see, for example, Bird et al., Science 242:423-426 (1988) and Huston et al., Proc. Natl. Acad. Sci. USA 85:5879-5883 (1988)).

A “diabody” consists of two chains, each chain comprising a heavy chain variable region connected to a light chain variable region on the same polypeptide chain connected by a short peptide linker, wherein the two regions on the same chain do not pair with each other but with complementary domains on the other chain to form a bispecific molecule. Methods of preparing diabodies are known in the art (see, e.g., Holliger P. et al., Proc. Natl. Acad. Sci. USA 90:6444-6448 (1993), and Poljak R. J. et al., Structure 2:1121-1123 (1994)).

“Domain antibodies” (dAbs) are small functional binding units of antibodies, corresponding to the variable regions of either the heavy or light chains of antibodies. Domain antibodies are well expressed in bacterial, yeast, and mammalian cell systems. Further details of domain antibodies and methods of production thereof are known in the art (see, for example, U.S. Pat. Nos. 6,291,158; 6,582,915; 6,593,081; WO04/003019 and WO03/002609).

A “nanobody” typically comprises a single variable domain and two constant domains (CH2 and CH3) and retains antigen-binding capacity of the original antibody. Nanobodies are derived from the heavy chains of an antibody. Nanobodies can be prepared by methods known in the art (see e.g., U.S. Pat. Nos. 6,765,087, 6,838,254, WO 06/079372).

“Unibodies” consist of one light chain and one heavy chain of an IgG4 antibody. Unibodies may be made by the removal of the hinge region of IgG4 antibodies. Further details of unibodies and methods of preparing them may be found in WO2007/059782.

The term “epitope” refers to the area or region of an antigen to which an antigen binding peptide (such as an antibody) specifically binds. “Epitope” is also referred to in the art as the “antigenic determinant”. An epitope generally consists of chemically active surface groupings of molecules such as amino acids or carbohydrate or sugar side chains. An epitope may be “linear” or “non-linear/conformational”. A protein epitope may comprise amino acid residues directly involved in the binding (also called immunodominant component of the epitope) and other amino acid residues, which are not directly involved in the binding, such as amino acid residues that are effectively blocked by the specifically antigen binding peptide (in other words, the amino acid residue is within the “footprint” of the specifically antigen binding peptide). Once a desired epitope is determined (e.g., by epitope mapping), antibodies to that epitope can be generated. The generation and characterization of antibodies may also provide information about desirable epitopes. From this information, it is then possible to screen antibodies for those which bind to the same epitope e.g., by conducting cross-competition studies to find antibodies that competitively bind with one another, i.e., the antibodies compete for binding to the antigen.

In particular, the term “epitope” includes the specific residues in a protein or peptide, e.g., ACE2, which are involved in the binding of an antibody to such protein or peptide as determined by known and accepted methods such as alanine scanning techniques or the use of various S protein portions with varying lengths.

Methods for determining the epitope of an antigen-binding protein, e.g., antibody or fragment or polypeptide, include alanine scanning mutational analysis, peptide blot analysis (Reineke Methods Mol. Biol. 248: 443-63(2004)), peptide cleavage analysis, crystallographic studies and NMR analysis. Epitope mapping is a method known which may be used in determining epitopes (DeLisser, Adhesion Protein Protocols. Methods Mol Biol. Vol. 96. pp. 11-20 (1999); Davidson and Doranz, Immunology. 143 (1): 13-20 (2014); Westwood and Hay eds., Epitope Mapping: A Practical Approach. Oxford, Oxfordshire: Oxford University Press (2001). In addition, methods such as epitope excision, epitope extraction and chemical modification of antigens can be employed (Tomer Prot. Sci. 9: 487-496 (2000)). Another method that can be used to identify the amino acids within a polypeptide with which an antigen-binding protein (e.g., antibody or fragment or polypeptide) interacts is hydrogen/deuterium exchange detected by mass spectrometry. See, e.g., Ehring Analytical Biochemistry 267: 252-259 (1999); Engen and Smith Anal. Chem. 73: 256A-265A (2001). An additional method for determining antibody epitopes is predicting protein epitopes through whole proteomes (Paull et al., PLoS ONE 14(9): e0217668 (2019)). Other methods such as yeast display, phage display (Mendonça, et al., PLOS ONE 11 (8): e0160544 (2016)) and limited proteolysis, provide high-throughput monitoring of antibody binding but lack resolution, especially for conformational epitopes (Flanagan, Genetic Engineering & Biotechnology News. 31 (10) (May 15, 2011).

The term “antibody derivative” or “derivative” of an antibody refers to a molecule that is capable of binding to the same antigen (i.e., human ACE2) that the antibody binds to and comprises an amino acid sequence of the antibody linked to an additional molecular entity. The amino acid sequence of the antibody that is contained in the antibody derivative may be the full-length antibody or may be any portion or portions of a full-length antibody. The additional molecular entity may be a biological or chemical molecule. Examples of additional molecular entities include chemical groups, peptides, proteins (such as enzymes, antibodies), amino acids, and chemical compounds. The additional molecular entity may be for use as a detection agent, marker label, therapeutic or pharmaceutical agent. The amino acid sequence of an antibody may be attached or linked to the additional entity by non-covalent association, chemical coupling, genetic fusion, or otherwise.

The term “host cell” refers to a cell into which an expression vector has been introduced. The term encompasses not only the particular subject cell but also the progeny of such a cell. Because certain modifications may occur in successive generations due to either environmental influences or mutation, such progeny may not be identical to the parent cell, but are still included within the scope of the term “host cell.”

The term “mammal” refers to any animal species of the Mammalian class. Examples of mammals include humans; laboratory animals such as rats, mice, simians and guinea pigs; domestic animals such as rabbits, cattle, sheep, goats, cats, dogs, horses, and pigs and the like.

The term “isolated nucleic acid” refers to a nucleic acid molecule of cDNA, or synthetic origin, or a combination thereof, which is separated from other nucleic acid molecules present in the natural source of the nucleic acid.

The term “Kd” or “K_{D}” refers to the equilibrium dissociation constant of a particular antibody-antigen interaction and is used to describe the binding affinity between a ligand (such as an antibody) and a protein (such as ACE2). The smaller the equilibrium dissociation constant, the more tightly bound the ligand is, or the higher the affinity between ligand and protein. A Kd can be measured by surface plasmon resonance, for example using the BIACORE 1 or the Octet system.

The term “variant”, as used herein in reference to a reference polypeptide (e.g., antibody, heavy chain, light chain, VH, or VL) refers to polypeptides whose amino acid sequences differ insubstantially from the reference polypeptide. In some embodiments, insubstantial differences include substitutions of 1, 2, 3, 4, 5, 6, 7, 8, 9, or 10 amino acids in a reference polypeptide, preferably, the substitutions do not adversely affect the properties of the reference polypeptide. In some embodiments, variants of a reference polypeptide include polypeptides comprising an amino acid sequence substantially identical to the reference polypeptide. In some embodiments, the sequence identity can be about 90%, 91%, 92%, 93%, 94%, 95%, 96%, 97%, 98%, 99% or higher. Percent identity can be determined for example by pairwise alignment using the default settings of the AlignX module of Vector NTI v.9.0.0 (Invitrogen, Carlsbad, Calif.). In some embodiments, the differences between a variant and a reference polypeptide involves one or more conservative amino acid substitutions with an amino acid having similar charge, hydrophobic, or stereo chemical characteristics in the antigen-binding site or in the framework without adversely altering the properties of the antibody. Conservative substitutions may also be made to improve antibody properties, for example stability or affinity. In some embodiments, 1, 2, 3, 4, 5, 6, 7, 8, 9, or 10 amino acid substitutions can be made to a reference VH or VL sequence. In some embodiments, 1, 2, or 3 substitutions are made to a reference VH or VL of an antibody described herein. Furthermore, any native residue in the polypeptide may also be substituted with alanine, as has been previously described for alanine scanning mutagenesis (MacLennan et al., Act Physiol. Scand. Suppl. 643:55-67 (1998); Sasaki et al., Adv. Biopsy's. 35:1-24 (1998)).

### Anti-Human ACE2 Antibodies and Antigen-Binding Fragments

Disclosed herein are anti-human ACE2 antibodies and antigen-binding fragments that inhibit ACE2 binding by a coronavirus.

Human angiotensin-converting enzyme 2 (ACE2) belongs to the angiotensin-converting enzyme family of dipeptidyl carboxydipeptidases and has considerable homology to human angiotensin 1 converting enzyme. The secreted protein catalyzes the cleavage of angiotensin I into angiotensin 1-9, and angiotensin II into the vasodilator angiotensin 1-7. ACE2 is known to be expressed in various human organs, and its organ- and cell-specific expression suggests that it may play a role in the regulation of cardiovascular and renal function, as well as fertility. Multiple splice variants have been found for this gene and the dACE2 (or MIRb-ACE2) splice variant has been found to be interferon inducible. Human ACE2 has a nucleotide sequence of SEQ ID NO: 93 and human ACE2 isoform 1 has an amino acid sequence of SEQ ID NO:94. The extracellular region of the ACE2 enzyme is composed of two domains. The first is a zinc metallopeptidase domain (residues 19-611). The second domain is located at the C-terminus (residues 612-740) and is 48% identical to human collectrin. The cytoplasmic tail contains several linear motifs such as LIR, PDZ-binding, PTB and endocytic sorting signal motifs that would allow interaction with proteins that mediate endocytic trafficking and autophagy.

Additionally, the encoded ACE2 protein is a functional receptor for the spike glycoprotein of the human coronavirus HCoV-NL63 and the human severe acute respiratory syndrome coronaviruses, SARS-CoV and SARS-CoV-2, the latter is the causative agent of coronavirus disease-2019 (COVID-19). Coronaviruses (CoVs) are a family of viruses that cause respiratory and intestinal illnesses in humans and animals. They usually cause mild colds in people but the emergence of the severe acute respiratory syndrome (SARS) epidemic in China in 2002-2003 and the Middle East respiratory syndrome (MERS) on the Arabian Peninsula in 2012 show they can also cause severe disease. Severe acute respiratory syndrome coronavirus 2 (SARS-CoV-2) is the coronavirus responsible for the outbreak of global pandemic of coronavirus disease (COVID-19), first identified in Wuhan, China. In some embodiments, the antibodies disclosed herein inhibit binding of human ACE2 by SARS-CoV-2, SARS-CoV, HCoV-NL63, HCoV-229E, HCoV-OC43, and/or HKU1, which are the human coronaviruses identified to date. In some embodiments, the antibodies disclosed herein inhibit binding of human ACE2 by any subgroup of the human coronavirus listed above, including alpha, beta, gamma, and delta subgroups.

The antibodies disclosed herein specifically bind to human ACE2. By “specifically” it means that the antibodies have a binding affinity to human ACE2 of at least about 10⁻⁹ M, 10⁻¹⁰M, 10⁻¹¹ M, or 10⁻¹² M; e.g., about 7.66 nM (expressed as K_{D}), as measured by an Octet® HTX biosensor, or by surface plasmon resonance, e.g., BIACORE™, or by solution-affinity ELISA.

In some embodiments, the antibody binds to the extracellular domain of human ACE2 (amino acids 1-740). In some embodiments, the antibody binds to the portion corresponding to amino acids Q24, T27, F28, D30, K31, H34, E35, E37, D38, Y41, Q42, L45, L79, M82, Y83, N90, Q325, E329, N330, K353, G354, R357 and R393 of the extracellular domain—this portion constituting the coronavirus binding site, separate from the enzymatic site of the protein. In some embodiments, the antibody binds to human ACE2 with a K_{D} of about 10⁻⁹ M, 10⁻¹⁰M, 10⁻¹¹ M, or 10⁻¹² M; e.g., about 7.66 nM.

Inhibition of binding to human ACE2 by a coronavirus inhibition can be determined, for example, through experiments described in Examples 3 and 4 of this disclosure. For example, to assess the antiviral activity, monoclonal antibodies can be incubated with target cells (such as Huh7.5cells), and thereafter, the target cells are infected with a coronavirus. IC₅₀ values can be determined as a parameter indicative of the effectiveness of the disclosed anti-ACE2 antibodies inhibition on coronavirus infectivity rates, providing for a quantitative measure indicating how much of the anti-ACE2 antibodies are necessary to inhibit the coronavirus mediated infection.

The antibodies disclosed herein inhibit binding of human ACE2 by a coronavirus, without affecting the enzymatic activity of human ACE2. The enzymatic activity of human ACE2 can be measured using an ACE2 Inhibitor Screening Assay Kit which is known in the art and commercially available from at least BPS Bioscience, as illustrated, for example, in Example 7 of this disclosure.

The antibodies disclosed herein inhibit binding of human ACE2 by a coronavirus without triggering endocytosis of human ACE2. The internalization of ACE2 can be measured through methodologies known in the art and is also illustrated in Example 8 of this disclosure.

In some embodiments, the antibody is an IgG, IgA, IgD, IgE or IgM antibody, such as IgG1, IgG2, IgG3 or IgG4 antibody. In some embodiments the antibody is an IgG1 or an IgG4 antibody.

The class (e.g., IgG, IgM, IgE, IgA, or IgD) and subclass (e.g., IgG1, IgG2, IgG3, or IgG4) of the anti-ACE2 antibodies may be determined by any suitable method such as by ELISA or Western Blot as well as other techniques. Alternatively, the class and subclass may be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies. The anti-ACE2 antibodies can be an IgG, an IgM, an IgE, an IgA, or an IgD molecule. For example, the anti-ACE2 antibodies can be an IgG that is an IgG1, IgG2, IgG3, or an IgG4 subclass. Thus, another embodiment of the disclosure provides a method for converting the class or subclass of an anti-ACE2 antibody to another class or subclass.

In some embodiments, the anti-ACE2 antibody is of the IgG4 isotype.

In some embodiments, the anti-ACE2 molecule is an antibody or antigen binding fragment wherein the antibody or antigen binding fragment thereof (i) binds to the extracellular domain of a human ACE2 protein, wherein binding of the human ACE2 protein by the antibody or antigen binding fragment thereof does not interfere with the enzymatic activity of the human ACE2 protein and does not induce internalization of the human ACE2 protein; and (ii) binds to the same epitope on the extracellular domain of the human ACE2 protein as a reference antibody or antigen-binding fragment thereof, or competes for binding to the extracellular domain of the human ACE2 protein with the reference antibody or antigen-binding fragment thereof. In some embodiments, the reference antibody or antigen-binding fragment thereof comprises a heavy chain variable domain comprising a complementarity determining region (CDR)1-H comprising the amino acid sequence GFTFSSYA (SEQ ID NO: 43) or a variant thereof, a CDR2-H comprising the amino acid sequence ISGSGDRT (SEQ ID NO: 44) or a variant thereof, and a CDR3-H comprising the amino acid sequence AKDWAMVGADAFDI (SEQ ID NO: 45) or a variant thereof. In some embodiments, the reference antibody or antigen-binding fragment thereof comprises a heavy chain variable domain comprising a CDR1-H comprising the amino acid sequence GFTFSSYA (SEQ ID NO: 48) or a variant thereof, a CDR2-H comprising the amino acid sequence ISISGGST (SEQ ID NO: 49) or a variant thereof, and a CDR3-H comprising the amino acid sequence VKDWYIVGADAFDI (SEQ ID NO: 50) or a variant thereof. In some embodiments, the reference antibody or antigen-binding fragment thereof comprises a heavy chain variable domain comprising a heavy chain variable domain comprising a CDR1-H comprising the amino acid sequence GFTFSSYA (SEQ ID NO: 53) or a variant thereof, a CDR2-H comprising the amino acid sequence INISGGST (SEQ ID NO: 54) or a variant thereof; and a CDR3-H comprising the amino acid sequence VKDWYIMGADAFDI (SEQ ID NO: 55) or a variant thereof. In some embodiments, the reference antibody or antigen-binding fragment thereof comprises a heavy chain variable domain comprising a heavy chain variable domain comprising a CDR1-H comprising the amino acid sequence GFTFSNYA (SEQ ID NO: 58) or a variant thereof, a CDR2-H comprising the amino acid sequence ISINGDRT (SEQ ID NO: 59) or a variant thereof; and a CDR3-H comprising the amino acid sequence AKDWAIVGADAFDV (SEQ ID NO: 60) or a variant thereof. In some embodiments, the reference antibody or antigen-binding fragment thereof comprises a heavy chain variable domain comprising a heavy chain variable domain comprising a CDR1-H comprising the amino acid sequence GFTFNIYA (SEQ ID NO: 63) or a variant thereof; a CDR2-H comprising the amino acid sequence FSGSRYNT (SEQ ID NO: 64) or a variant thereof; and a CDR3-H comprising the amino acid sequence of AKEAVAGQFDY (SEQ ID NO: 65) or a variant thereof. In some embodiments, the reference antibody or antigen-binding fragment thereof comprises a heavy chain variable domain comprising a heavy chain variable domain comprising a CDR1-H comprising the amino acid sequence DGSISGYY (SEQ ID NO: 68) or a variant thereof; a CDR2-H comprising the amino acid sequence IHYSGTT (SEQ ID NO: 69) or a variant thereof; and a CDR3-H comprising the amino acid sequence ARGPVFWYFDL (SEQ ID NO: 70) or a variant thereof. In some embodiments, the reference antibody or antigen-binding fragment thereof comprises a heavy chain variable domain comprising a heavy chain variable domain comprising a CDR1-H comprising the amino acid sequence DGSISGYY (SEQ ID NO: 73) or a variant thereof; a CDR2-H comprising then amino acid sequence IYYSGTT (SEQ ID NO: 74) or a variant thereof; and a CDR3-H comprising the amino acid sequence ARGPVFWYFDL (SEQ ID NO: 75) or a variant thereof. In some embodiments, the reference antibody or antigen-binding fragment thereof comprises a heavy chain variable domain comprising a heavy chain variable domain comprising a CDR1-H comprising the amino acid sequence GFTFSSYS (SEQ ID NO: 78) or a variant thereof; a CDR2-H comprising the amino acid sequence ITNGINNI (SEQ ID NO: 79) or a variant thereof; and a CDR3-H comprising the amino acid sequence ARYYYYYGMIDV (SEQ ID NO: 80) or a variant thereof. In some embodiments, the reference antibody or antigen-binding fragment thereof comprises a heavy chain variable domain comprising a heavy chain variable domain comprising a CDR1-H comprising the amino acid sequence GGSITSYY (SEQ ID NO: 83) or a variant thereof; a CDR2-H comprising the amino acid sequence IFSSGIT (SEQ ID NO: 84) or a variant thereof; and a CDR3-H comprising the amino acid sequence ARHDGFGWFDP (SEQ ID NO: 85) or a variant thereof. In some embodiments, the reference antibody or antigen-binding fragment thereof comprises a heavy chain variable domain comprising a heavy chain variable domain comprising a CDR1-H comprising the amino acid sequence GFSLSTPGVA (SEQ ID NO: 88) or a variant thereof, a CDR2-H comprising the amino acid sequence IFWNDDE (SEQ ID NO: 89) or a variant thereof; and a CDR3-H comprising the amino acid sequence AHLALYWYFDF (SEQ ID NO: 90) or a variant thereof. In some embodiments, the reference antibody or antigen-binding fragment thereof comprises a light chain variable domain comprising a CDR1-L comprising the amino acid sequence QGISNY (SEQ ID NO: 46) or a variant thereof, a CDR2-L comprising the amino acid sequence AAS or a variant thereof, and a CDR3-L comprising the amino acid sequence LQHSYYPYT (SEQ ID NO: 47) or a variant thereof. In some embodiments, the reference antibody or antigen-binding fragment thereof comprises a light chain variable domain comprising a CDR1-L comprising the amino acid sequence QGISNY (SEQ ID NO: 51) or a variant thereof, a CDR2-L comprising the amino acid sequence ATS or a variant thereof, and a CDR3-L comprising the amino acid sequence LQHSNYPYT (SEQ ID NO: 52) or a variant thereof. In some embodiments, the reference antibody or antigen-binding fragment thereof comprises a light chain variable domain comprising a CDR1-L comprising the amino acid sequence QGISNY (SEQ ID NO: 56) or a variant thereof, a CDR2-L comprising the amino acid sequence ATS or a variant thereof, and a CDR3-L comprising the amino acid sequence LQHSNYPYT (SEQ ID NO: 57) or a variant thereof. In some embodiments, the reference antibody or antigen-binding fragment thereof comprises a light chain variable domain comprising a CDR1-L comprising the amino acid sequence QVISNY (SEQ ID NO: 61) or a variant thereof, a CDR2-L comprising the amino acid sequence AGS or a variant thereof, and a CDR3-L comprising the amino acid sequence LQHNNYPYT (SEQ ID NO: 62) or a variant thereof. In some embodiments, the reference antibody or antigen-binding fragment thereof comprises a light chain variable domain comprising a CDR1-L comprising the amino acid sequence QSISSW (SEQ ID NO: 66) or a variant thereof, a CDR2-L comprising the amino acid sequence KAS or a variant thereof, and a CDR3-L comprising the amino acid sequence QQYNTYSRT (SEQ ID NO: 67) or a variant thereof. In some embodiments, the reference antibody or antigen-binding fragment thereof comprises a light chain variable domain comprising a CDR1-L comprising the amino acid sequence SGSINNNY (SEQ ID NO: 71) or a variant thereof, a CDR2-L comprising the amino acid sequence DDN or a variant thereof, and a CDR3-L comprising the amino acid sequence QSYDSSSRV (SEQ ID NO: 72) or a variant thereof. In some embodiments, the reference antibody or antigen-binding fragment thereof comprises a light chain variable domain comprising a CDR1-L comprising the amino acid sequence SGSISNSY (SEQ ID NO: 76) or a variant thereof, a CDR2-L comprising the amino acid sequence DDN or a variant thereof, and a CDR3-L comprising the amino acid sequence QSYDSSSRV (SEQ ID NO: 77) or a variant thereof. In some embodiments, the reference antibody or antigen-binding fragment thereof comprises a light chain variable domain comprising a CDR1-L comprising the amino acid sequence SLRNYY (SEQ ID NO: 81) or a variant thereof, a CDR2-L comprising the amino acid sequence GKN or a variant thereof, and a CDR3-L comprising the amino acid sequence NSRDSGGNHVV (SEQ ID NO: 82). In some embodiments, the reference antibody or antigen-binding fragment thereof comprises a light chain variable domain comprising a CDR1-L comprising the amino acid sequence QSVSSSY (SEQ ID NO: 86) or a variant thereof, a CDR2-L comprising the amino acid sequence GAS or a variant thereof, and a CDR3-L comprising the amino acid sequence QQYGSSPRT (SEQ ID NO: 87) or a variant thereof. In some embodiments, the reference antibody or antigen-binding fragment thereof comprises a light chain variable domain comprising a CDR1-L comprising the amino acid sequence SSDVGSYNL (SEQ ID NO: 91) or a variant thereof, a CDR2-L comprising the amino acid sequence EGS ( ) or a variant thereof, and a CDR3-L comprising the amino acid sequence CSYAGSNIVV (SEQ ID NO: 92) or a variant thereof.

In some embodiments of the disclosure, variants of the anti-ACE2 antigen-binding proteins, e.g., antibodies and antigen-binding fragments thereof of the present disclosure, include a heavy chain immunoglobulin or variable region thereof having at least 80% (e.g., 80%, 85%, 90%, 91%, 92%, 93%, 94%, 95%, 96%, 97%, 98%, or 99%) amino acid sequence identity to the amino acids set forth in SEQ ID NOs: 6, 10, 14, 18, 22, 26, 30, 34, 38, or 42; and/or a light chain immunoglobulin or variable region thereof having at least 80% (e.g., 80%, 85%, 90%, 91%, 92%, 93%, 94%, 95%, 96%, 97%, 98%, or 99%) amino acid sequence identity to the amino acids set forth in SEQ ID NOs: 4, 8, 12, 16, 20, 24, 28, 32, 36, or 40.

The present disclosure includes antigen-binding proteins that compete for binding to human ACE2, with an antibody specifically disclosed herein, e.g., 05B04, 05D06, 05E10, 05G01, 05H02, 2G7A1, 2F6A6, 4A12A4, 2C12H3, 1C9H1, or 05B04/05D06, or an antigen-binding fragment thereof. The term “competes” as used herein, refers to antibody or antigen-binding fragment thereof that binds to an antigen (e.g., human ACE2) and inhibits or blocks the binding of another antibody or antigen-binding fragment thereof to the antigen. The term also includes competition between two antigen-binding proteins e.g., antibodies, in both orientations, i.e., a first antibody that binds and blocks binding of second antibody and vice versa. In some embodiments, the first antibody and second antibody may bind to the same epitope. Alternatively, the first and second antibodies may bind to epitopes that are not identical but overlap, wherein binding of one inhibits or blocks the binding of the second antibody, e.g., via steric hindrance. Competition between antibodies may be measured by methods known in the art through competitive binding assays.

In some embodiments, a variant of an antibody or antigen-binding fragment retains the ability to specifically bind to human ACE2 of the antibody or antigen-binding fragment, e.g., retains at least 50% of its ACE2 binding activity. In some embodiments, a variant of an antibody or antigen-binding fragment possesses at least 70%, 80%, 90%, 95% or 100% or more of the human ACE2 binding affinity as the antibody antigen-binding fragment. Variants of an antibody or antigen-binding fragment of the disclosure may include conservative or non-conservative amino acid substitutions (referred to as “conservative variants” or “function conserved variants” of the antibody) that do not substantially alter its biologic activity.

A “variant” of an immunoglobulin chain, e.g., 05B04, 05D06, 05E10, 05G01, 05H02, 2G7A1, 2F6A6, 4A12A4, 2C12H3, 1C9H1, or 05B04/05D06 VH, VL, HC or LC, refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein (e.g., any of SEQ ID NOs: 1, 2, 4, 6, 8, 10, 12, 14, 16, 18, 20, 22, 24, 26, 28, 30, 32, 34, 36, 38, 40, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, or 102); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

A “variant” of a polynucleotide refers to a polynucleotide comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence that is set forth herein (e.g., any of SEQ ID NOs: 3, 5, 7, 9, 11, 13, 15, 17, 19, 21, 23, 25, 27, 29, 31, 33, 35, 37, 39, and/or 41); when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1, −2; gap costs: linear).

The following references relate to BLAST algorithms often used for sequence analysis: BLAST ALGORITHMS: Altschul et al. FEBS J. 272(20): 5101-5109 (2005); Altschul, S. F., et al., J. Mol. Biol. 215:403-410 (1990); Gish, W., et al., Nature Genet. 3:266-272 (1993); Madden, T. L., et al., Meth. Enzymol. 266:131-141 (1996); Altschul, S. F., et al., Nucleic Acids Res. 25:3389-3402 (1997); Zhang, J., et al., Genome Res. 7:649-656 (1997); Wootton et al., Comput. Chem. 17:149-163 (1993); Hancock et al., Comput. Appl. Biosci. 10:67-70 (1994); ALIGNMENT SCORING SYSTEMS: Dayhoff, et al., “A model of evolutionary change in proteins.” in Atlas of Protein Sequence and Structure, vol. 5, suppl. 3. M. O. Dayhoff (ed.), pp. 345-352, Natl. Biomed. Res. Found., Washington, D.C. (1978); Schwartz, et al., “Matrices for detecting distant relationships.” in Atlas of Protein Sequence and Structure, vol. 5, suppl. 3.” M. O. Dayhoff (ed.), pp. 353-358, Natl. Biomed. Res. Found., Washington, D.C. (1978); Altschul, J. Mol. Biol. 219:555-565 (1991); States, et al., Methods 3:66-70 (1991); Henikoff, et al., Proc. Natl. Acad. Sci. USA 89:10915-10919 (1992); Altschul, et al., J. Mol. Evol. 36:290-300 (1993); ALIGNMENT STATISTICS: Karlin, et al., Proc. Natl. Acad. Sci. USA 87:2264-2268 (1990); Karlin, et al., Proc. Natl. Acad. Sci. USA 90:5873-5877 (1993); Dembo, A., et al., Ann. Prob. 22:2022-2039 (1994); and Altschul, “Evaluating the statistical significance of multiple distinct local alignments.” in Theoretical and Computational Methods in Genome Research (S. Suhai, ed.), pp. 1-14, Plenum, N.Y. (1997).

In addition, an anti-ACE2 antibody may include a polypeptide comprising an amino acid sequence that is set forth herein except for one or more (e.g., 1, 2, 3, 4, 5, 6, 7, 8, 9 or 10) mutations such as, for example, missense mutations (e.g., conservative substitutions), non-sense mutations, deletions, or insertions. For example, the present disclosure includes anti-ACE2 antigen-binding proteins which include an immunoglobulin light chain (or VL) variant comprising the amino acid sequence set forth in SEQ ID NOs: 4, 8, 12, 16, 20, 24, 28, 32, 36, or 40 but having one or more of such mutations and/or an immunoglobulin heavy chain (or VH) variant comprising the amino acid sequence set forth in SEQ ID NOs: 6, 10, 14, 18, 22, 26, 30, 34, 38, or 42 but having one or more of such mutations. In an embodiment of the disclosure, an anti-ACE2 antigen-binding protein includes an immunoglobulin light chain variant comprising CDR-L1, CDR-L2 and CDR-L3 wherein one or more (e.g., 1 or 2 or 3) of such CDRs has one or more of such mutations (e.g., conservative substitutions) and/or an immunoglobulin heavy chain variant comprising CDR-H1, CDR-H2 and CDR-H3 wherein one or more (e.g., 1 or 2 or 3) of such CDRs has one or more of such mutations (e.g., conservative substitutions).

The phrase that an antibody binds “substantially” the same epitope as a reference antibody means that the epitope binding site for the antibody comprises at least 50%, 60%, 70%, 80%, 90%, or more of the amino acid residues on the antigen that constitute the epitope binding site of the reference antibody. An antibody that binds the same epitope as a reference antibody means that the epitope binding site for the antibody comprises the same amino acid residues on the antigen that constitute the epitope binding site of the reference antibody. An antibody binds the same or substantially the same epitope as a reference antibody competes in binding to the antigen.

The identification of one or more antibodies that bind(s) to substantially the same epitope as the monoclonal antibodies described herein can be readily determined using alanine scanning. Additionally, any one of variety of immunological screening assays in which antibody competition can be assessed. A number of such assays are routinely practiced and well known in the art (see, e.g., U.S. Pat. No. 5,660,827, issued Aug. 26, 1997, which is specifically incorporated herein by reference). It will be understood that actually determining the epitope to which an antibody described herein binds is not in any way required to identify an antibody that binds to the same or substantially the same epitope as the monoclonal antibody described herein.

Embodiments of the present disclosure also include antigen-binding proteins, e.g., anti-ACE2 antibodies and antigen-binding fragments thereof, that comprise immunoglobulin VHS and VLS; or HCs and LCs, which comprise a variant amino acid sequence having 80% or more (e.g., 80%, 85%, 90%, 91%, 92%, 93%, 94%, 95%, 96%, 97%, 98%, or 99%) overall amino acid sequence identity or similarity to the amino acid sequences of the corresponding VHS, VLS, HCs or LCs specifically set forth herein, but wherein the CDR-L1, CDR-L2, CDR-L3, CDR-H1, CDR-H2 and CDR-H3 of such immunoglobulins are not variants and comprise the amino acid sequences specifically set forth herein. Thus, in such embodiments, the CDRs within variant antigen-binding proteins are not, themselves, variants.

A “conservatively modified variant” or a “conservative substitution”, e.g., of an immunoglobulin chain set forth herein, refers to a variant wherein there is one or more substitutions of amino acids in a polypeptide with other amino acids having similar characteristics (e.g. charge, side-chain size, hydrophobicity/hydrophilicity, backbone conformation and rigidity, etc.). Such changes can frequently be made without significantly disrupting the biological activity of the antibody or fragment. Those of skill in the art recognize that, in general, single amino acid substitutions in non-essential regions of a polypeptide do not substantially alter biological activity (see, e.g., Watson et al. Molecular Biology of the Gene, The Benjamin/Cummings Pub. Co., p. 224 (4th Ed.) (1987)). In addition, substitutions of structurally or functionally similar amino acids are less likely to significantly disrupt biological activity. The present disclosure includes anti-ACE2 antigen-binding proteins comprising such conservatively modified variant immunoglobulin chains.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. Science 256: 1443 45 (1992).

In some embodiments, the anti-ACE2 antibodies or antigen binding fragments thereof compete for binding to the same epitope as the coronavirus spike proteins. In some embodiments, the antibody or antigen binding fragment thereof binds to the same epitope on the extracellular domain of the human ACE2 protein as does the receptor binding domain (RBD) of the SARS-CoV-2 spike protein, or the RBD of any other member of the betacoronavirus genus or sarbecovirus sub-genus that uses ACE2 to enter cells or any member of the alphacoronavirus genus. A “receptor-binding domain” or “RBD” is a short immunogenic fragment from a virus that binds to a specific endogenous receptor sequence in order for the virus to gain entry into cells and cause infection. RBD is a critical component of the viral spike glycoprotein that is found on coronaviruses. Specifically, RBDs refer to a part of the ‘spike’ glycoprotein (S-domain) which is needed to interact with endogenous receptors to facilitate membrane fusion and delivery to the cytoplasm. In some embodiments, the anti-ACE2 antibodies or antigen binding fragments thereof bind to the same epitope on the extracellular domain of the human ACE2 protein as does the RBD of the alphacoronavirus human coronavirus HCoV-NL-63.

A competitive binding assay can be used to measure the binding of a labeled ligand to a target protein in the presence of a second, competing but unlabeled ligand. Such an assay can be used to assess qualitative binding information as well as relative affinities for two or binding molecules for one target. Such competition assays are known in the art. In some embodiments, the antibodies or antigen binding fragments thereof inhibit coronavirus in substantially the same effectiveness as a reference antibody. Inhibition of coronavirus can be measured as described in this disclosure and in the art.

### Multispecific Antibodies

Multispecific antibodies are monoclonal antibodies that have binding specificities for at least two different sites (i.e., different antigenic epitopes). In some embodiments, bispecific antibodies may bind to two different epitopes of human ACE2.

In some embodiments, the antibodies or antigen-binding fragments disclosed herein are bispecific antibodies that comprise a first variable domain and a second variable domain, wherein the first and second variable domains are different and are selected from the group consisting of
(a) a heavy chain variable domain comprising a complementarity determining region (CDR)1-H comprising the amino acid sequence GFTFSSYA (SEQ ID NO: 43) or a variant thereof, a CDR2-H comprising the amino acid sequence ISGSGDRT (SEQ ID NO: 44) or a variant thereof, and a CDR3-H comprising the amino acid sequence AKDWAMVGADAFDI (SEQ ID NO: 45) or a variant thereof, and a light chain variable domain comprising a CDR1-L comprising the amino acid sequence QGISNY (SEQ ID NO: 46) or a variant thereof, a CDR2-L comprising the amino acid sequence AAS or a variant thereof, and a CDR3-L comprising the amino acid sequence LQHSYYPYT (SEQ ID NO: 47) or a variant thereof;
(b) a heavy chain variable domain comprising a CDR1-H comprising the amino acid sequence GFTFSSYA (SEQ ID NO: 48) or a variant thereof, a CDR2-H comprising the amino acid sequence ISISGGST (SEQ ID NO: 49) or a variant thereof, and a CDR3-H comprising the amino acid sequence VKDWYIVGADAFDI (SEQ ID NO: 50) or a variant thereof, and a light chain variable domain comprising a CDR1-L comprising the amino acid sequence QGISNY (SEQ ID NO: 51) or a variant thereof, a CDR2-L comprising the amino acid sequence ATS (SEQ ID NO: 52) or a variant thereof, and a CDR3-L comprising the amino acid sequence LQHSNYPYT (SEQ ID NO: 52) or a variant thereof;
(c) a heavy chain variable domain comprising a CDR1-H comprising then amino acid sequence GFTFSSYA (SEQ ID NO: 53) or a variant thereof; a CDR2-H comprising the amino acid sequence INISGGST (SEQ ID NO: 54) or a variant thereof, and a CDR3-H comprising the amino acid sequence VKDWYIMGADAFDI (SEQ ID NO: 55) or a variant thereof, and a light chain variable domain comprising a CDR1-L comprising the amino acid sequence QGISNY (SEQ ID NO: 56) or a variant thereof, a CDR2-L comprising the amino acid sequence ATS or a variant thereof; and a CDR3-L comprising the amino acid sequence LQHSNYPYT (SEQ ID NO: 57) or a variant thereof;
(d) a heavy chain variable domain comprising a CDR1-H comprising the amino acid sequence GFTFSNYA (SEQ ID NO: 58) or a variant thereof, a CDR2-H comprising the amino acid sequence ISINGDRT (SEQ ID NO: 59) or a variant thereof, and a CDR3-H comprising the amino acid sequence AKDWAIVGADAFDV (SEQ ID NO: 60) or a variant thereof; and a light chain variable domain comprising a CDR1-L comprising the amino acid sequence QVISNY (SEQ ID NO: 61) or a variant thereof, a CDR2-L comprising the amino acid sequence AGS or a variant thereof; and a CDR3-L comprising the amino acid sequence LQHNNYPYT (SEQ ID NO: 62) or a variant thereof;
(e) a heavy chain variable domain comprising a CDR1-H comprising the amino acid sequence GFTFNIYA (SEQ ID NO: 63) or a variant thereof; a CDR2-H comprising the amino acid sequence FSGSRYNT (SEQ ID NO: 64) or a variant thereof; and a CDR3-H comprising the amino acid sequence of AKEAVAGQFDY (SEQ ID NO: 65) or a variant thereof; and a light chain variable domain comprising a CDR1-L comprising the amino acid sequence QSISSW (SEQ ID NO: 66) or a variant thereof; a CDR2-L comprising the amino acid sequence KAS or a variant thereof; and a CDR3-L comprising the amino acid sequence QQYNTYSRT (SEQ ID NO: 67) or a variant thereof;
(f) heavy chain variable domain comprising a CDR1-H comprising the amino acid sequence DGSISGYY (SEQ ID NO: 68) or a variant thereof; a CDR2-H comprising the amino acid sequence IHYSGTT (SEQ ID NO: 69) or a variant thereof; and a CDR3-H comprising the amino acid sequence ARGPVFWYFDL (SEQ ID NO: 70) or a variant thereof; and a light chain variable domain comprising a CDR1-L comprising the amino acid sequence SGSINNNY (SEQ ID NO: 71) or a variant thereof; a CDR2-L comprising the amino acid sequence DDN or a variant thereof; and a CDR3-L comprising the amino acid sequence QSYDSSSRV (SEQ ID NO: 72) or a variant thereof;
(g) a heavy chain variable domain comprising a CDR1-H comprising the amino acid sequence DGSISGYY (SEQ ID NO: 73) or a variant thereof; a CDR2-H comprising then amino acid sequence IYYSGTT (SEQ ID NO: 74) or a variant thereof; and a CDR3-H comprising the amino acid sequence ARGPVFWYFDL (SEQ ID NO: 75) or a variant thereof; and a light chain variable domain comprising a CDR1-L comprising the amino acid sequence SGSISNSY (SEQ ID NO: 76) or a variant thereof; a CDR2-L comprising the amino acid sequence DDN or a variant thereof; and a CDR3-L comprising the amino acid sequence QSYDSSSRV (SEQ ID NO: 77) or a variant thereof;
(h) a heavy chain variable domain comprising a CDR1-H comprising the amino acid sequence GFTFSSYS (SEQ ID NO: 78) or a variant thereof; a CDR2-H comprising the amino acid sequence ITNGINNI (SEQ ID NO: 79) or a variant thereof; and a CDR3-H comprising the amino acid sequence ARYYYYYGMDV (SEQ ID NO: 80) or a variant thereof; and a light chain variable domain comprising a CDR1-L comprising the amino acid sequence SLRNYY (SEQ ID NO: 81) or a variant thereof; a CDR2-L comprising the amino acid sequence GKN or a variant thereof; and a CDR3-L comprising the amino acid sequence NSRDSGGNHVV (SEQ ID NO: 82);
(i) a heavy chain variable domain comprising a CDR1-H comprising the amino acid sequence GGSITSYY (SEQ ID NO: 83) or a variant thereof; a CDR2-H comprising the amino acid sequence IFSSGIT (SEQ ID NO: 84) or a variant thereof; and a CDR3-H comprising the amino acid sequence ARHDGFGWFDP (SEQ ID NO: 85) or a variant thereof; and a light chain variable domain comprising a CDR1-L comprising the amino acid sequence QSVSSSY (SEQ ID NO: 86) or a variant thereof; a CDR2-L comprising the amino acid sequence GAS or a variant thereof; and a CDR3-L comprising the amino acid sequence QQYGSSPRT (SEQ ID NO: 87) or a variant thereof; and/or
(j) a heavy chain variable domain comprising a CDR1-H comprising the amino acid sequence GFSLSTPGVA (SEQ ID NO: 88) or a variant thereof; a CDR2-H comprising the amino acid sequence IFWNDDE (SEQ ID NO: 89) or a variant thereof; and a CDR3-H comprising the amino acid sequence AHLALYWYFDF (SEQ ID NO: 90) or a variant thereof; and a light chain variable domain comprising a CDR1-L comprising the amino acid sequence SSDVGSYNL (SEQ ID NO: 91) or a variant thereof; a CDR2-L comprising the amino acid sequence EGS or a variant thereof; and a CDR3-L comprising the amino acid sequence CSYAGSNIVV (SEQ ID NO: 92) or a variant thereof.

### VH/VL Pairing

In accordance with the present disclosure, the VH CDR1, CDR2, and CDR3 sequences and VL CDR1, CDR2, and CDR3 sequences can be “mixed and matched”. For example, CDRs from different anti-ACE2 antibodies originally identified can be mixed and matched. The binding of such “mixed and matched” antibodies to ACE2 can be tested using the binding assays described in the Examples (e.g., ELISAs, Biacore analysis). In some embodiments, when VH CDR sequences are mixed and matched, the CDR1, CDR2 and/or CDR3 sequence from a particular VH sequence is replaced with structurally similar CDR sequence(s). Likewise, when VL CDR sequences are mixed and matched, the CDR1, CDR2 and/or CDR3 sequence from a particular VL sequence typically is replaced with a structurally similar CDR sequence(s).

### Fc Variants

The Fc region of an antibody, i.e., the terminal ends of the heavy chains of antibody spanning domains CH2, CH3 and a portion of the hinge region, is limited in variability and is involved in effecting the physiological roles played by the antibody. The effector functions attributable to the Fc region of an antibody vary with the class and subclass of antibody and include binding of the antibody via the Fc region to a specific Fc receptor (“FcR”) on a cell which triggers various biological responses. Several strategies exist for improving antibody Fc-mediated effector functions. Multiple point mutations have been identified that improve binding affinity of Fc for specific FcγRs. In some instances, a single FcγR, such as FcγRIIIa is the receptor of interest. Directed evolution, alanine scanning, or structure-guided design have been used to identify these mutations. An example of these mutations is the DLE (Ser239Asp/Ile332Glu/Ala330Leu) set of mutations (Ahmed, et al., J Struct Biol. 194:78-89(2016)). These mutations improve ADCC activity. Additionally, mutations can be inserted that improve antibody circulation in vivo. LS mutations, Met428Leu/Asn434Ser, are one example of antibody half-life extension mutations (Saunders, Conceptual Approaches to Modulating Antibody Effector Functions and Circulation Half-Life, Frontiers in Immunology, 10 (2019)). Antibody effector functions can be enhanced by glycoengineering the Fc domain. The Fc domain contains a N-linked glycan at position 297. Expression of antibody in wildtype cells results in a fucosylated complex glycans present at N297. However, specialized cells have been created with fucosyltransferase knocked out, which results in afucosylated glycans at Asn297 (Shields, et al., J Biol Chem. 277:26733-40 (2002)). Antibodies with afucosylated glycans exhibit up to 50-fold more potent ADCC than the same antibody with a fucosylated glycan at Asn297 (Shields, et al., J Biol Chem. 277:26733-40 (2002)). Antibody effector functions can be improved by expanding the breadth of Fc receptors capable of interacting with Fc. To improve antibody effector function the Fc of a single antibody can be engineered to bind to Fc receptors for multiple antibody isotypes. This concept has led to the design of cross-isotype IgGA antibodies where the IgG1 CH2 al loop residues 245-258 and the IgG1 CH3 domain were exchanged with the structurally analogous regions of IgA (Kelton, et al., Chem Biol. 21:1603-9 (2014)). Regions can be combined to create a chimeric cross-isotype Fe where the cross-isotype Fc is capable of binding to FcγRI and FcαRI, hence either of these Fc receptors can be used to recruit diverse effector cells to target cells.

In some embodiments, the anti-ACE2 antibodies or antigen binding fragments thereof comprise substitutions at L234 L235 (LALA) that abolish FcR-gamma interaction, and/or substitutions at M428 N434 (LS) that enhance interaction with the neonatal Fc receptor to prolong antibody half-life in human. In some embodiments, the anti-ACE2 antibodies or antigen binding fragments thereof comprise the substitutions/mutations above for improving antibody Fc-mediated effector functions.

### Compositions and Methods for Making the Antibodies

The disclosure further provides a nucleic acid molecule encoding an antibody disclosed herein. Further provided is a nucleic acid molecule encoding a heavy chain variable region, a heavy chain, a light chain variable region, or a light chain of a humanized antibody of the disclosure.

One aspect of the disclosure provides an isolated nucleic acid encoding an immunoglobulin chain or variable region thereof of the ACE2 antibody according to the disclosure. The disclosure also includes an isolated nucleic acid that encodes an ACE2 antibody polypeptide, fragment, homolog, analog, or derivative thereof. In some embodiments, the nucleic acid molecule comprises the nucleotide sequence of a naturally occurring allelic nucleic acid variant. In another embodiment, the nucleic acid encodes a variant polypeptide, wherein the variant polypeptide has the polypeptide sequence of a naturally occurring polypeptide variant. In some embodiments, the nucleic acid molecule differs by a single nucleotide from a nucleotide that encodes an ACE2 antibody polypeptide, fragment, homolog, analog, or derivative thereof. In some embodiments, the nucleic acid molecule differs by 1, 2, 3, 4, 5, 6, 7, 8, 9, or 10 nucleotides from a nucleic acid sequence that encodes an ACE2 antibody polypeptide, fragment, homolog, analog, or derivative thereof. In some embodiments, an isolated nucleic acid molecule of the disclosure comprises a nucleic acid molecule that is a complement of the nucleotide sequence shown in SEQ ID NOS: 3, 5, 7, 9, 11, 13, 15, 17, 19, 21, 23, 25, 27, 29, 31, 33, 35, 37, 39, and/or 41. Some embodiments of the disclosure provide a vector comprising an isolated nucleic acid that encodes an ACE2 antibody, fragment, homolog, analog, or derivative thereof.

Other embodiments of the disclosure provide a host cell comprising an isolated nucleic acid that encodes an ACE2 antibody, fragment, homolog, analog, or derivative thereof. Some embodiments of the disclosure provide a host cell comprising a vector comprising an isolated nucleic acid that encodes an ACE2 antibody, fragment, homolog, analog, or derivative thereof.

Eukaryotic and prokaryotic host cells, including mammalian cells, may be used as hosts for expression of an anti-IL36R antigen-binding protein (e.g., antibody or antigen-binding fragment thereof). Such host cells are well known in the art and many are available from the American Type Culture Collection (ATCC). These host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells, Expi 293 cells, and a number of other cell lines. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia, Pichia pastoris, Pichia finlandica, Pichia trehalophila, Pichia koclamae, Pichia membranaefaciens, Pichia minuta (Ogataea minuta, Pichia lindnen), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenuda polymorpha, Kluveromyces sp., Kluveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. The present disclosure includes an isolated host cell (e.g., a CHO cell or any type of host cell set forth above) comprising an antigen-binding protein, such as 05B04, 05D06, 05E10, 05G01, 05H02, 2G7A1, 2F6A6, 4A12A4, 2C12H3, 1C9H1, or 05B04/05D06, or an antigen-binding fragment thereof; and/or a polynucleotide encoding one or more immunoglobulin chains thereof.

Some embodiments of the disclosure are directed to methods by which to produce anti-ACE2 antigen-binding proteins, such as an antibody or antigen-binding fragment thereof, and such methods are known in the art. One example of a method for recombinant production of antibodies is disclosed in U.S. Pat. No. 4,816,567.

Some embodiments of the disclosure include introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei Additionally, nucleic acid molecules may be introduced into mammalian cells through the use of viral vectors. Methods of transforming cells are well known in the art. See, for example, U.S. Pat. Nos. 4,399,216, 4,912,040; 4,740,461 and 4,959,455. The present disclosure includes recombinant methods for making an anti-ACE2 antigen-binding protein, such as an antibody or antigen-binding fragment thereof of the present disclosure, or an immunoglobulin chain thereof, comprising (i) introducing one or more nucleic acids encoding an immunoglobulin chain of antibody or antigen-binding fragment thereof (e.g., including the nucleotide sequence in any one or more of SEQ ID NOS: 3, 5, 7, 9, 11, 13, 15, 17, 19, 21, 23, 25, 27, 29, 31, 33, 35, 37, 39, and/or 41; or a variant thereof) encoding light and/or heavy immunoglobulin chains of the antigen-binding protein, e.g., 05B04, 05D06, 05E10, 05G01, 05H02, 2G7A1, 2F6A6, 4A12A4, 2C12H3, 1C91-1, or 05B04/05D06 For example, wherein the polynucleotide is in a vector, and/or integrated into a host cell chromosome and/or is operably linked to a promoter (ii) culturing the host cell (e.g., CHO or Pichia or Pichia pastoris) in a medium under conditions favorable to express the immunoglobulin chain(s) and, (iii) optionally, isolating the immunoglobulin chain or antibody or antigen-binding fragment thereof from the host cell and/or medium in which the host cell is grown. When making an antigen-binding protein (e.g., antibody or antigen-binding fragment) comprising more than one immunoglobulin chain, e.g., an antibody that comprises two heavy immunoglobulin chains and two light immunoglobulin chains, co-expression of the chains in a single host cell leads to association of the chains, e.g., in the cell or on the cell surface or outside the cell if such chains are secreted, so as to form the antigen-binding protein (e.g., antibody or antigen-binding fragment) The methods of the present disclosure include those wherein only a heavy immunoglobulin chain or only a light immunoglobulin chain or both (e.g., any of those discussed herein including mature fragments and/or variable domains thereof) are expressed in a cell. Such single chains can be useful, for example, as intermediates when expressing an antibody or antigen-binding fragment including that chain.

For example, the present disclosure also includes anti-ACE2 antigen-binding proteins, such as antibodies and antigen-binding fragments thereof, comprising a heavy chain immunoglobulin (or variable domain thereof or comprising the CDRs thereof) encoded by a polynucleotide comprising the nucleotide sequences set forth in SEQ ID NO: 5, 9, 13, 17, 21, 25, 29, 33, 37, and/or 41; and a light chain immunoglobulin (or variable domain thereof or comprising the CDRs thereof) encoded by the nucleotide sequence set forth in SEQ ID NOS: 3, 7, 11, 15, 19, 23, 27, 31, 35, and/or 39 which are the product of such production methods, and, optionally, the purification methods set forth herein. For example, in an embodiment of the disclosure, the product of the method is an anti-ACE2 antigen-binding protein which is an antibody or fragment comprising a heavy chain immunoglobulin or V_{H} comprising the amino acid sequence set forth in SEQ ID NO: 6, 10, 14, 18, 22, 26, 30, 34, 38, or 42; and a light chain immunoglobulin or V_{L} comprising the amino acid sequence set forth in SEQ ID NOS. 4, 8, 12, 16, 20, 24, 28, 32, 36, or 40.

One embodiment of the disclosure is a method for making an anti-ACE2 antigen-binding protein, e.g., antibody or antigen-binding fragment thereof, includes a method of purifying the antigen-binding protein, e.g., by column chromatography, precipitation and/or filtration. The current disclosure also includes the product of such a method.

### Pharmaceutical Compositions and Therapeutic Methods

This disclosure further provides a pharmaceutical composition or formulation comprising an antibody or antigen-binding fragment thereof disclosed herein and a pharmaceutically acceptable carrier.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

To prepare pharmaceutical compositions of the anti-ACE2 antigen-binding proteins, e.g., antibodies and antigen-binding fragments thereof (e.g., 05B04, 05D06, 05E10, 05G01, 05H02, 2G7A1, 2F6A6, 4A12A4, 2C12H3, 1C9H1, or 05B04/05D06), antigen-binding protein is admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY: Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y. In an embodiment of the disclosure, the pharmaceutical composition is sterile. Such compositions are part of the present disclosure.

Pharmaceutical compositions of the present disclosure include pharmaceutically acceptable carriers, diluents, excipients and/or stabilizers, such as, for example, water, buffering agents, stabilizing agents, preservatives, isotonifiers, non-ionic detergents, antioxidants and/or other miscellaneous additives.

The scope of the present disclosure includes desiccated, e.g., freeze-dried, compositions comprising an anti-ACE2 antigen-binding protein, e.g., antibody or antigen-binding fragment thereof (e.g. 05B04, 05D06, 05E10, 05G01, 05H02, 2G7A1, 2F6A6, 4A12A4, 2C12H3, 1C9H1, or 05B04/05D06), or a pharmaceutical composition thereof that includes a pharmaceutically acceptable carrier but substantially lacks water.

This disclosure additionally provides a method for treating or preventing infection caused by coronavirus in a subject in need thereof, comprising administering to the subject a therapeutically effective amount of an antibody or antigen-binding fragment thereof disclosed herein.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the disclosure are used to delay development of a disease or to slow the progression of a disease. For example, by “treating an infection caused by a coronavirus”, it means that one or more signs and/or symptoms and/or clinical indicia of infection caused by the coronavirus regresses or is eliminated and/or the progression thereof is inhibited (e.g., the disease in the subject is stabilized, reduced or eliminated); and by “preventing an infection caused by a coronavirus”, it include complete and partial prevention, e.g., the occurrence of an infection by a coronavirus is reduced or eliminated.

In some embodiments, the present disclosure provides methods for treating or preventing an infection caused by coronavirus by administering a therapeutically effective amount of anti-ACE2 antigen-binding protein, e.g., antibody or antigen-binding fragment, (e.g., 05B04, 05D06, 05E10, 05G01, 05H02, 2G7A1, 2F6A6, 4A12A4, 2C12H3, 1C9H1, or 05B04/05D06) to a subject (e.g., a human) in need of such treatment or prevention.

An effective or therapeutically effective dose of anti-ACE2 antigen-binding protein, e.g., antibody or antigen-binding fragment (e.g., 05B04, 05D06, 05E10, 05G01, 05H02, 2G7A1, 2F6A6, 4A12A4, 2C12H3, 1C9H1, or 05B04/05D06), for treating or preventing a coronavirus-mediated disease refers to the amount of the antibody or fragment sufficient to alleviate one or more of the clinical indicia, signs and/or symptoms of the disease in the treated subject, whether by inducing the regression or elimination of such indicia, signs and/or symptoms or by inhibiting the progression of such indicia, signs and/or symptoms. The dose amount may vary depending upon the age and the size of a subject to be administered, target disease, conditions, route of administration, and the like. In an embodiment of the disclosure, an effective or therapeutically effective dose of antibody or antigen-binding fragment thereof of the present disclosure, for treating or preventing coronavirus mediated disease, e.g., in an adult human subject, is about 1 mg/kg or more, e.g., about 1 mg/kg to about 25 mg/kg. Depending on the severity of the infection, the frequency and the duration of the treatment can be adjusted. In certain embodiments, the antigen-binding protein of the present disclosure can be administered at an initial dose, followed by one or more secondary doses. In certain embodiments, the initial dose may be followed by administration of a second or a plurality of subsequent doses of antigen-binding protein in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

The mode of administration of an antigen-binding protein or composition thereof can vary. Routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal or intra-arterial.

The present disclosure includes combinations including an anti-ACE2 antigen-binding protein, e.g., antibody or antigen-binding fragment thereof of the present disclosure (e.g., 05B04, 05D06, 05E10, 05G01, 05H02, 2G7A1, 2F6A6, 4A12A4, 2C12H3, 1C9H1, or 05B04/05D06), in association with one or more further therapeutic agents. The anti-ACE2 antigen-binding protein and the further therapeutic agent can be in a single composition or in separate compositions.

Methods for treating or preventing a coronavirus-mediated disease in a subject in need of treatment or prevention by administering an anti-ACE2 antigen-binding protein, e.g., 05B04, 05D06, 05E10, 05G01, 05H02, 2G7A1, 2F6A6, 4A12A4, 2C12H3, 1C9H1, and 05B04/05D06, in association with a further therapeutic agent are part of the present disclosure.

The term “in association with” indicates that components, an anti-ACE2 antigen-binding protein, e.g., antibody or antigen-binding fragment thereof of the present disclosure, along with another agent such as methotrexate, can be formulated into a single composition, e.g., for simultaneous delivery, or formulated separately into two or more compositions (e.g., a kit including each component). Each component can be administered to a subject at a different time than when the other component is administered; for example, each administration may be given non-simultaneously (e.g., separately or sequentially) at intervals over a given period of time. Moreover, the separate components may be administered to a subject by the same or by a different route.

### EXAMPLES

The steps of the method described in the various examples disclosed herein are sufficient to carry out the methods of the present disclosure. Thus, in an example, a method consists essentially of a combination of the steps of the methods disclosed herein. In another example, a method consists of such steps.

The following examples are presented to illustrate the present disclosure. The examples are not intended to be limiting in any manner.

### Example 1. Plasmid Construction

Plasmids expressing the spike proteins from SARS-CoV, SARS-CoV-2 (Wuhan-hu-1, Beta (B.1.351), Delta (B.1.617.2), and Omicron (B.1.1.529) variants), the horseshoe bat (Rhinolophus affinis) coronavirus bCoV-RaTG13, the pangolin (Manis javanica) coronaviruses from Guangdong, China (pCoV-GD) and Guanxi, China (pCoV-GX) have been previously described. Human codon-optimized cDNAs encoding spike proteins from the rufous horseshoe bat (Rhinolophus sinicus) coronaviruses Rs4231 and Rs7327 were generated using GenSmart™ Codon Optimization, synthesized by IDT gBlocks, and inserted into the pCR3.1 expression vector using NheI and XbaI and NEBuilder® HiFi DNA Assembly.

### Spike-NanoLuc Luciferase Expression Plasmids

To construct the plasmids expressing NanoLuc-fused to conformationally stabilized versions of the SARS-CoV-2 Wuhan-hu-1 or Omicron variant spike proteins, the HexaPro (6P) modified cDNAs was fused at its C-terminus with DNA encoding a trimerization domain, a GGSGG spacer sequence, NanoLuc luciferase (NLuc), a human rhinovirus 3C protease cleavage site and a polyhistidine tag (8×His). This cDNA, termed (S-6P-NanoLuc), was inserted into the pCR3.1 expression vector.

hACE2 and hACE-2 Fc Expression Plasmids

To construct the plasmid expressing catalytically inactive, His-tagged, soluble ectodomain of ACE2 (1-740 aa), H374N and H378N substitutions were introduced by overlap extension PCR into a hACE2 cDNA and, an His-tag was fused to its C-terminus, and the purified PCR product was inserted into the pCAGGS expression vector (ACE2-1-740aa-His). To construct the expression plasmid encoding hACE2 (1-740aa)-NanoLuc-8×His, the ACE2 1-740aa and NanoLuc-8×His fragments were PCR amplified using ACE2-1-740aa-His and S-6P-NanoLuc as templates, respectively, followed by Gibson assembly and insertion into the pCR3.1 expression vector.

### Human Antibody Expression Plasmids

DNA encoding the variable regions of the heavy (VH) and light (VL) from hybridomas was PCR amplified from hybridomas. For the 05B04 and 05D06 antibodies, the DNA sequences encoding VH and VL were human codon-optimized using GenSmart™ Codon Optimization, synthesized by IDT. For each antibody, DNA encoding VH were fused to cDNA encoding the Fc domain of human IgG1, in which Fc domain was modified to include the substitutions at L234 L235 (LALA) that abolish FcR-gamma interaction, and substitutions at M428 N434 (LS) that enhance interaction with the neonatal Fc receptor to prolong antibody half-life in humans. To construct the expression plasmids for and subcloned into heavy and light chain antibody expression vectors (von Boehmer L et al., 2016) using NEBuilder® HiFi DNA Assembly.

### Example 2. Protein Expression and Purification

To express the monomeric, His-tagged human ACE2 extracellular domain (residues 1-740) used as immunogen, Expi293 cells were transfected with the expression plasmid ACE2-1-740aa-8×His using ExpiFectamine 293 (ThermoFisher Scientific). Four days later, the supernatant was filtered with 0.22-um membrane filter and loaded on Ni-NTA agarose (Qiagen) and, after washing, ACE2 (1-740aa) proteins were eluted with 200 mM imidazole in PBS. Dimeric, Fc-fused ACE2 extracellular domain was also expressed in Expi293 cells in the same way. The secreted proteins in supernatant were first incubated with Protein G Sepharose 4 Fast Flow overnight at 4° C., loaded into column, and, after washing, eluted with 0.1 M glycine, pH 2.9 into tubes containing 1/10th volume of 1 M Tris, pH8.0.

For expression of monoclonal antibodies, Expi293 cells were transfected with the corresponding light chain and heavy chain expression plasmids at the ratio of 1:1 using ExpiFectamine 293. Four days later, the antibodies in the supernatant were purified through Protein G Sepharose 4 Fast Flow and eluted with 0.1 M glycine, pH 2.9 as described above.

To express S-6P-NanoLuc proteins, Expi293 cells were transfected with S-6P-NanoLuc expression plasmids that included for the original Wuhan-hu-1 or Omicron spike variants using ExpiFectamine 293. Three days later, the supernatant was harvested and loaded on Ni-NTA agarose and, after thorough wash, S-6P-NanoLuc proteins were released after HRV 3C protease (TaKaRa) treatment overnight at 4° C.

To express the His-tagged ACE2 (1-740aa)-NanoLuc proteins, Expi293 cells were transfected with expression plasmids ACE2(1-740aa)-NanoLuc-8×His using ExpiFectamine 293. Four days later, the supernatant was harvested and loaded on Ni-NTA agarose and, after washing, ACE2(1-740aa)-NanoLuc proteins were eluted with 200 mM imidazole in PBS. All recombinant proteins, including purified antibodies, were dialyzed against PBS before used in further experiments.

### Example 3. Generation of Sarbecovirus Spike-Bearing Pseudotyped Virus and Infectivity Inhibition Assay

To generate HIV-1 virions pseudotyped with Sarbecovirus spikes, including SARS-CoV, SARS-CoV-2 (Wuhan-hu-1, Beta, Delta, and Omicron variants), pangolin coronavirus pCoV-GD, pangolin coronavirus pCoV-GX, bat coronavirus RaTG13a, bat SARSr-CoV 4231, and bat SARSr-CoV 7327, ten million 293T cells in a 15-cm dish were transfected with 25 g of an HIV-1 envelope-deficient proviral plasmid expressing NanoLuc along with 7.5 g of spike expression plasmids, in which the C terminal 19aa was truncated (A19). Cells were washed twice with PBS the next morning and virions were harvested at 48 hours post transfection, filtered (0.22 m), and purified by Lenti-X Concentrator (TaKaRa).

To measure the infectivity, viral stocks were two-fold serially diluted and added to Huh7.5 cells, a SARS-CoV-2 susceptible cell line, in 96-well plates seeded one day prior to infection. Infection assay performed as described in Schmidt et al. J Exp Med 2020 Nov. 2; 217(11):e20201181. PMID: 32692348, except that the anti-ACE2 antibodies were preincubated with cells rather than viruses prior to infection. The 05B04 LC/05D06 HC antibody is a hybrid antibody generated by coexpression of the 05B04 light chain with the heavy chain of a clonally related antibody, 05D06. Cells were then harvested at 48 hours post infection for measuring NanoLuc activity using the Nano-Glo Luciferase Assay System and GloMax® Navigator Microplate Luminometer (Promega).

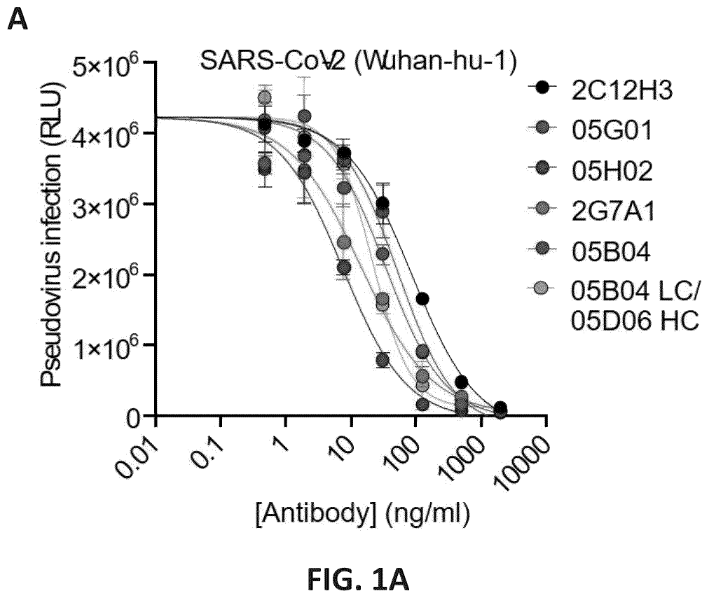

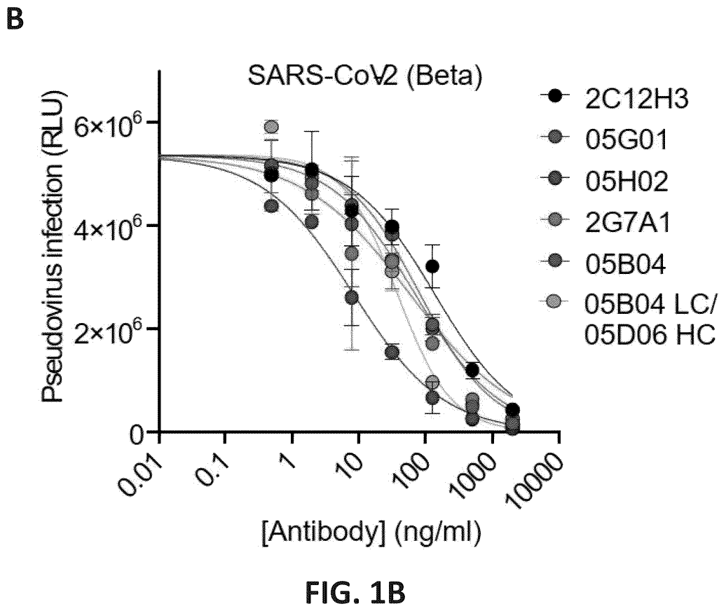

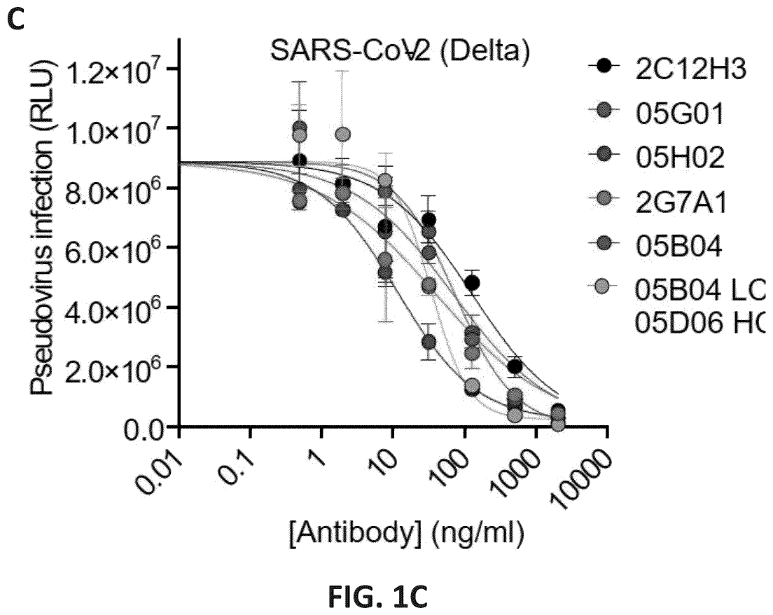

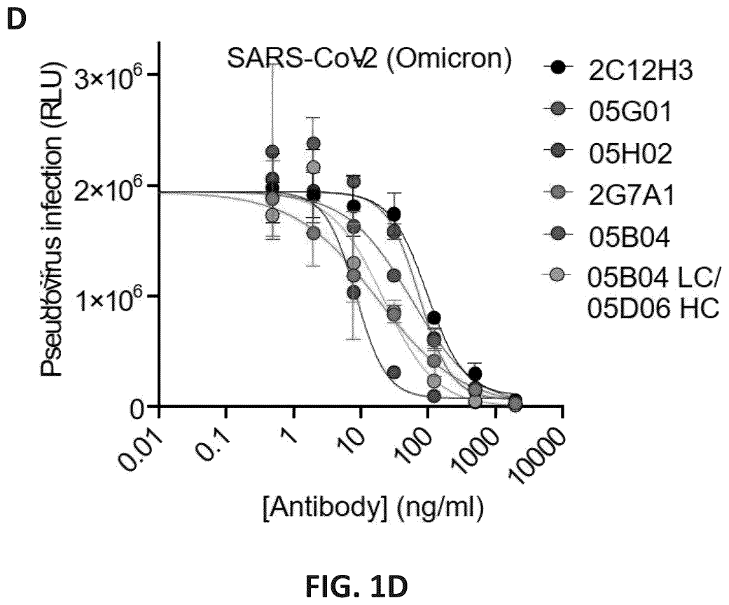

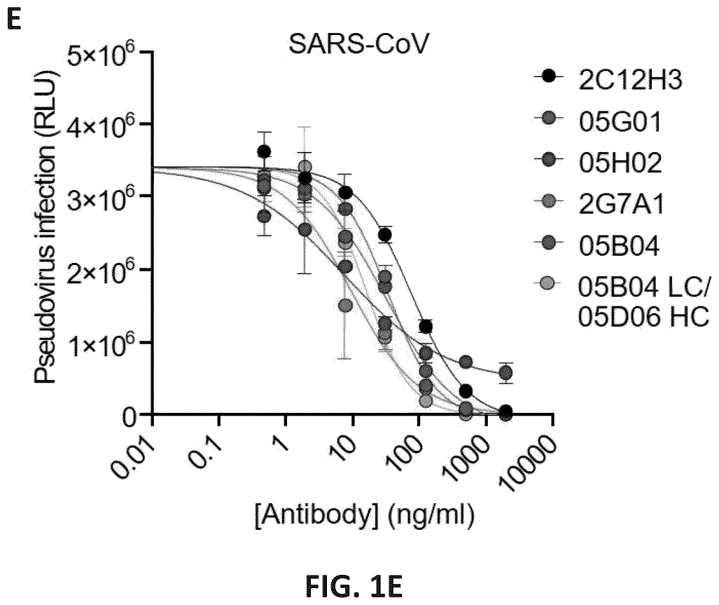

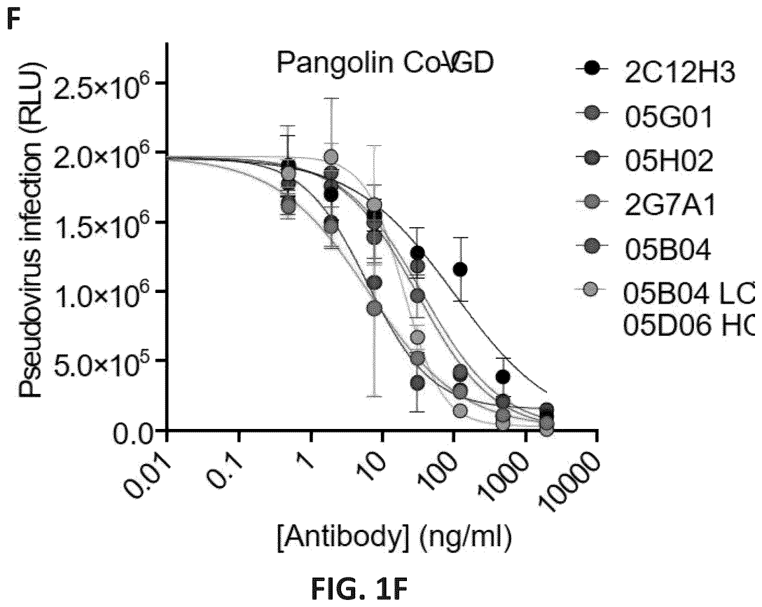

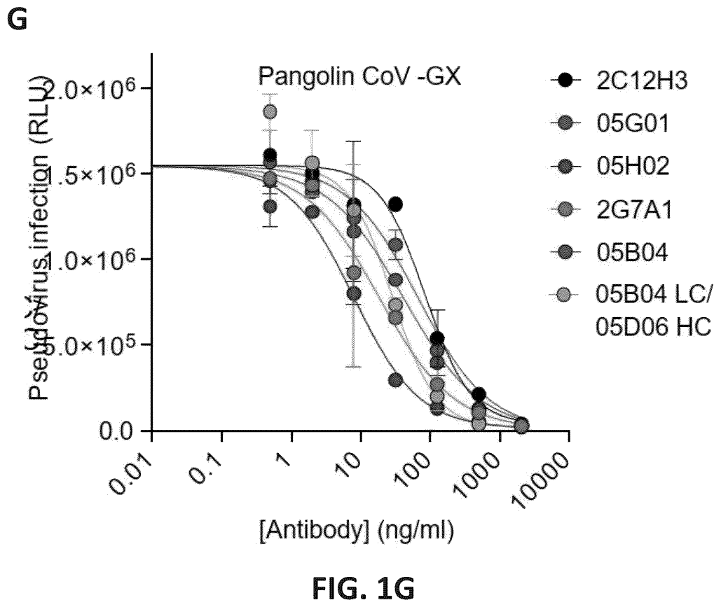

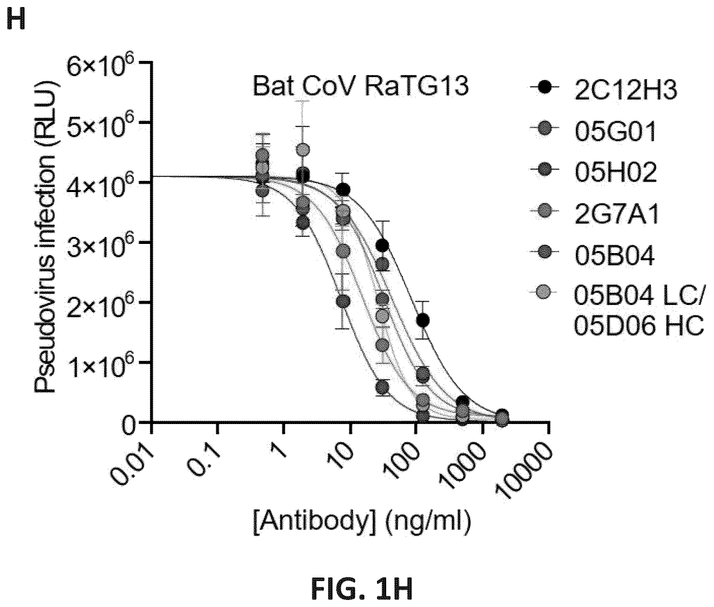

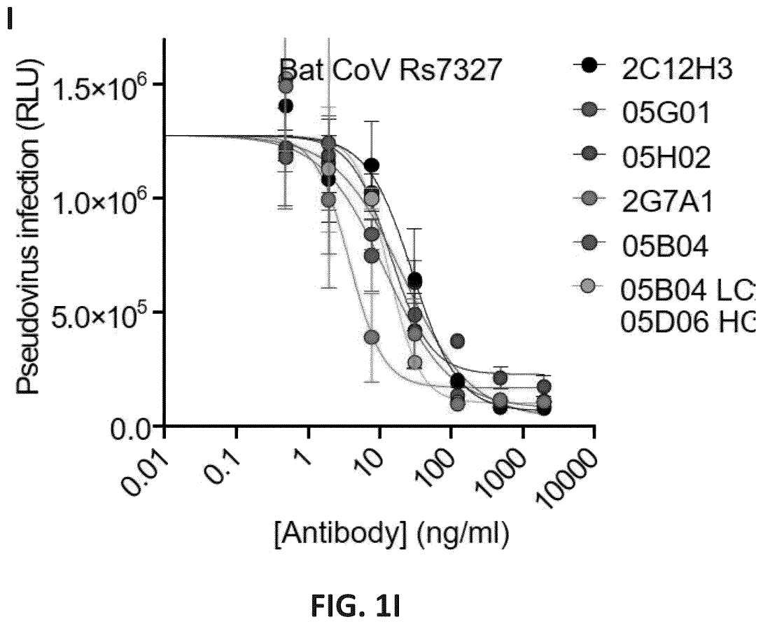

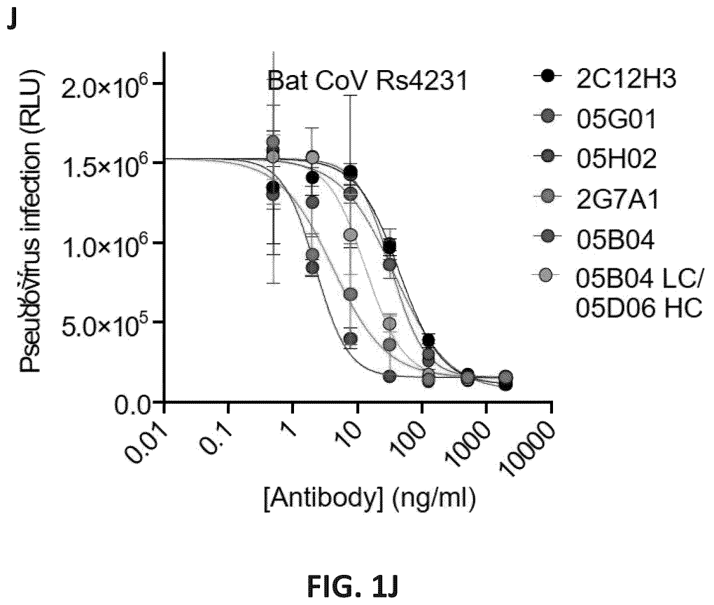

To assess the antiviral activity, monoclonal antibodies, beginning with 2 μg/ml were four-fold serially diluted in 96-well plates over seven dilutions and incubated with Huh7.5 target cells for 1 hour at 37° C. Thereafter, Huh7.5 cells were infected with sarbecovirus spike pseudotyped viruses. Cells were harvested 48 hours post infection for and NanoLuc luciferase activity measured in infected cells as described above. FIG. 1A-JJ show the inhibition curves for anti-hACE2 antibody inhibition of pseudotyped virus infection determined using HIV-1 based pseudoviruses and Huh7 target cells where FIG. 1A-1D show the inhibition of SARS-CoV-2 variants Wuhan-hu-1, Beta, Delta, and Omicron; FIG. 1E shows inhibition for SARS-CoV; FIG. 1F-1G show inhibition for pangolin sarbecoviruses; and FIG. 1H-1J show inhibition curves for bat sarbecoviruses.

The IC₅₀ values for anti-hACE2 antibody inhibition of pseudotyped virus infection were determined using HIV-1 based pseudoviruses and Huh7 target cells as described in Schmidt et al. J Exp Med 2020 Nov. 2; 217(11):e20201181. PMID: 32692348, with the exception of the anti-hACE2 antibodies were preincubated with cells rather than viruses prior to infection. Table 1 shows the IC₅₀ results of the assay where the 05B04 LC/05D06 HC is a hybrid antibody generated by coexpression of the light chain of the 05B04 antibody with the heavy chain of the clonally related 05D06 antibody.

TABLE 1
IC₅₀ values (ng/ml) for antiviral activity of fully human anti-ACE2 antibodies

| | | | | | | 05B04 |
|---|---|---|---|---|---|---|
| | | | | | | LC/ |
| | | | | | | 05D06 |
| | 2C12H3 | 05G01 | 05H02 | 2G7A1 | 05B04 | HC |
| SARS-CoV-2 (Wuhan-Hu-1) | 86.14 | 52.68 | 7.513 | 14.21 | 34.78 | 21.71 |
| SARS-CoV-2 (Beta) | 138.5 | 82.06 | 8.366 | 58.61 | 70.13 | 37.78 |
| SARS-CoV-2 (Delta) | 125.9 | 71.97 | 11.27 | 39.26 | 67.81 | 33.62 |
| SARS-CoV-2 (Omicron) | 98.13 | 74.17 | 8.49 | 17.97 | 56.65 | 22.13 |
| SARS-CoV | 76.57 | 34.67 | 7.563 | 9.602 | 31.54 | 16.26 |
| Pangolin CoV-GD | 102.9 | 31.28 | 6.329 | 6.595 | 37.03 | 20.35 |
| Pangolin CoV-GX | 82.89 | 66.09 | 7.823 | 18.23 | 41.01 | 28.06 |
| Bat CoV RaTG13 | 85.39 | 43.29 | 7.345 | 15.27 | 32 | 25.83 |
| Bat CoV Rs7327 | 29.47 | 21.85 | 14.37 | 3.848 | 12.32 | 13.47 |
| Bat CoV Rs4231 | 44.14 | 33.02 | 2.221 | 4.139 | 37.6 | 13.38 |

### Example 4. Inhibition of Authentic SARS-CoV-2 Infection by Anti-Human ACE2 Antibodies

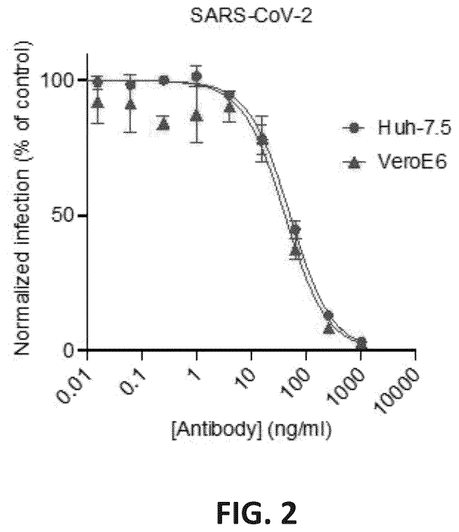

FIG. 2. Inhibition of authentic SARS-CoV-2 infection by anti-human ACE2 antibodies shown through inhibition curves for anti-hACE2 antibody (05B04) inhibition of authentic SARS-CoV-2 infection determined using SARS-CoV-2, USA_WA/2020 P3, and Vero E6 or Huh7 target cells. The anti-hACE2 antibody inhibition of virus infection was determined using authentic SARS-CoV-2 and Huh7 or Vero target cells as described in Schmidt et al. J Exp Med 2020 Nov. 2; 217(11):e20201181. PMID: 32692348, with the exception of the anti-hACE2 antibodies were preincubated with cells rather than viruses prior to infection.

### Example 5. Specific Binding to hACE2 by Human Anti ACE2 Antibodies

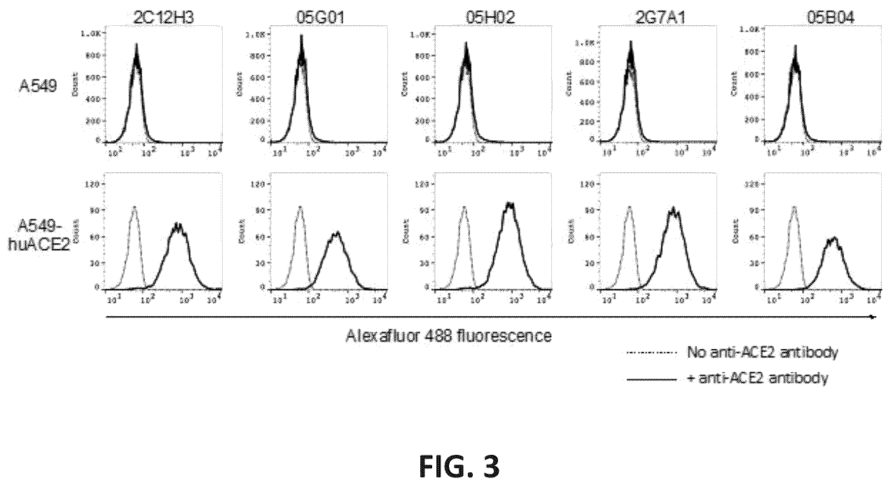

FIG. 3 shows the flow cytometry analysis results representing the specific binding to hACE2 by human anti-ACE2 antibodies. To evaluate the ability of anti-ACE2 antibodies to detect cell surface ACE2 by flow cytometry, A549 cells (human alveolar basal epithelial cells) were engineered to express human ACE2 ectodomain. The cells expressing human ACE2 (105) were detached from plates with 10 mM EDTA in PBS and incubated in the absence or the presence of human anti-ACE2 antibodies (2 μg/ml) for 2 hours at 4° C. After washing, the cells were then incubated with AlexaFluor™ 488 conjugated goat anti-human IgG (ThermoFisher Scientific). Flow cytometry was performed using Attune® NxT Acoustic Focusing Cytometer (ThermoFisher Scientific). The same procedure was applied to parental, unmodified A549 cells as a negative control for nonspecific cell surface binding.

### Example 6. Binding to hACE2 by Human Anti ACE2 Antibodies

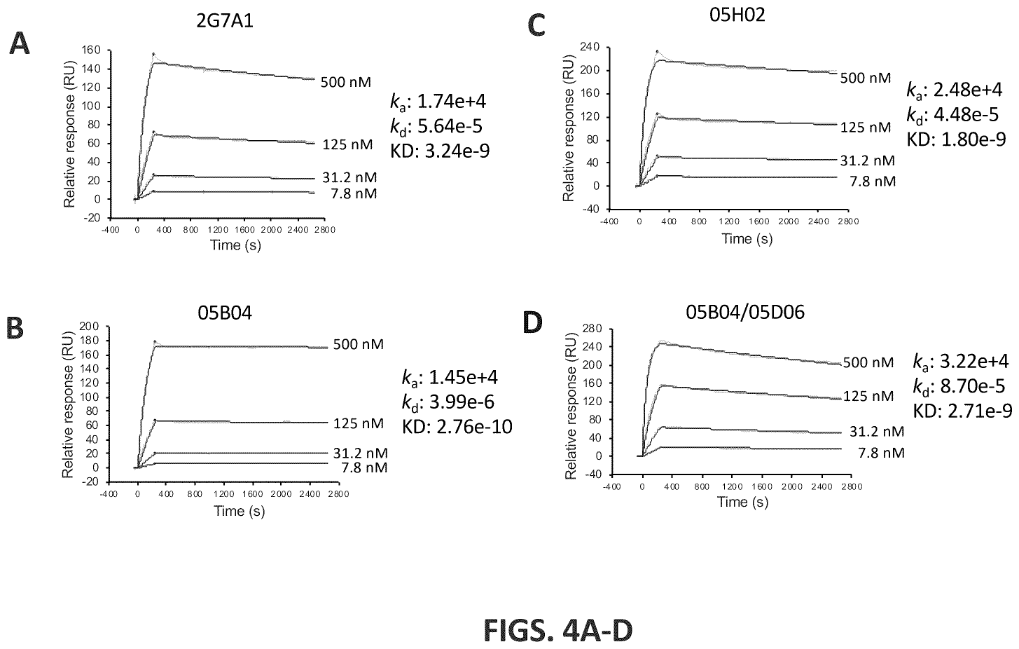

SPR experiments were performed using a Biacore 8K instrument (GE Healthcare). Human antibodies 2G7A1, 05B04, 05H02 and hybrid antibody 05B04 LC/05D06 HC were captured onto a Series S Sensor ship Protein G (Cytiva) at the concentration of 20 nM at the flow rate of 10 μl/min for 60 s. Flow cell one was kept empty and used as a negative control. A concentration series of His-tagged ACE2 1-740aa proteins (4-fold dilutions from a maximum concentration of 500 nM) was injected at 30 μl/min for 240 s followed by a dissociation phase of 2400 s at a flow rate of 30 μl/min. Binding reactions were allowed to reach equilibrium and KD values were calculated from the ratio of association and dissociation constants (KD=kd/ka), which were derived using a 1:1 binding model that was globally fit to all curves in a data set. Flow cells were regenerated with 10 mM glycine pH 1.5 at a flow rate of 30 μl/min for 30 s. FIG. 4 shows the relative response of human each anti-ACE2 antibody and their K_{D}.

### Example 7. Anti-Human ACE2 Antibodies do not Inhibit ACE2 Enzymatic Activity

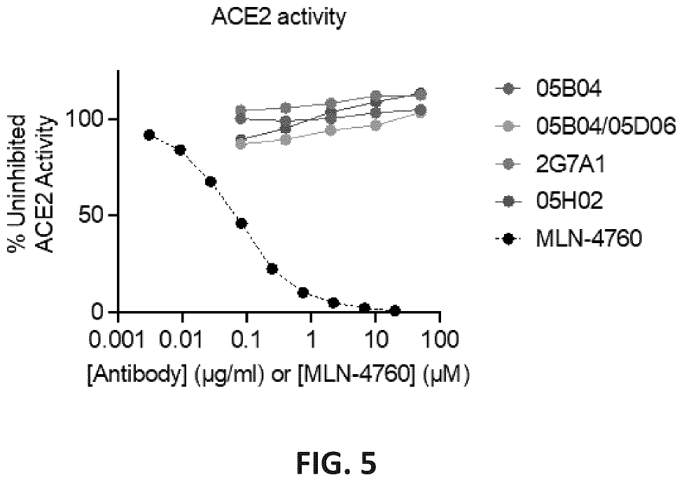

To measure the effect of anti-ACE2 antibodies on the catalytic activity of ACE2, various concentrations of 2G7A1, 05B04, 051102, and 05B04LC/05D06HC (2 μg/ml, 10 μg/ml, 50 μg/ml) were mixed with ACE2 (0.2 μg/ml). ACE2 enzymatic activity was measured using the ACE2 Inhibitor Screening Assay Kit (BPS Bioscience) following the normal protocol. The intensity of the fluorescent product of the ACE2 reaction product was detected at 555 nm/585 nm (excitation/emission) with Clariostar Plus Microplate Reader (BMG Labtech). MLN-4760 (Sigma, #5306160001) served as a positive control ACE2 inhibitor. FIG. 5 shows the enzymatic activity of ACE2 when 2G7A1, 05B04, 05H02, and 05B04LC/05D06HC antibodies were added at 0.08 to 50 μg/ml as well as the MLN-4760 positive control of an ACE2 inhibitor. These results show that the anti-human ACE2 antibodies do not inhibit the enzymatic activity of ACE2.

### Example 8. Anti-Human ACE2 Antibodies do not Induce ACE2 Internalization

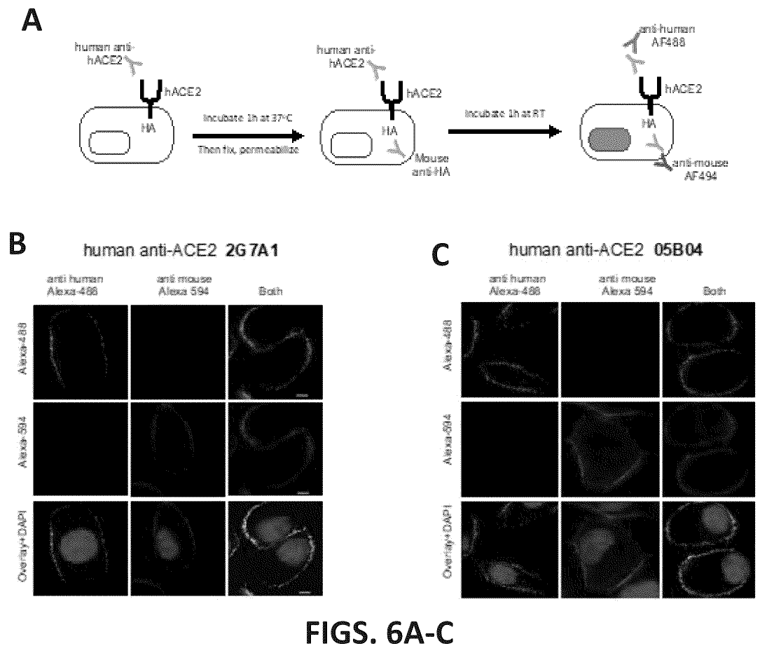

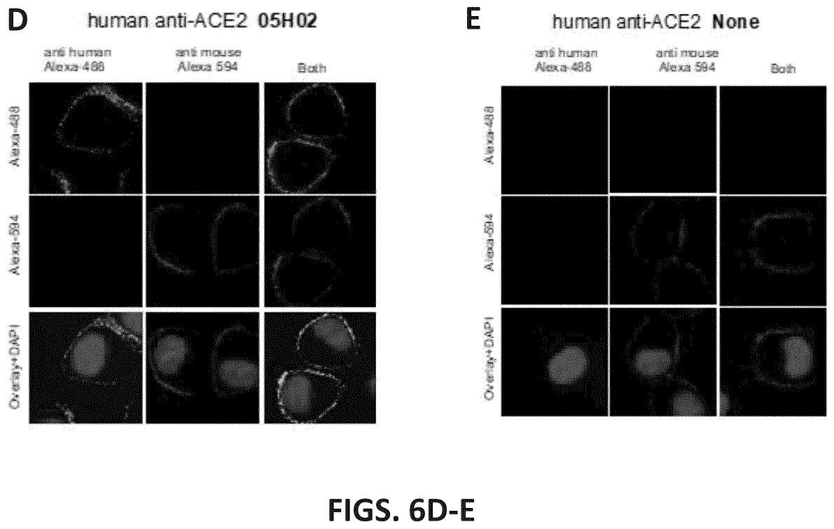

FIG. 6A shows a schematic of the internalization experiment used to determine whether the anti-ACE2 antibodies induced ACE2 internalization upon binding, live A549 cells expressing a human ACE2 receptor with an HA-epitope tag appended to its intracellular C-terminus were incubated with anti-human ACE2 antibodies (1 μg/ml) for 2 hours at 37° C. Cells were then fixed with 4% PFA/PBS, treated with 10 mM glycine, and permeabilized with 0.1% Triton X-100. Total human ACE2-HA was then detected with mouse anti-HA.11 antibody (1 μg/ml). The internalization of the ACE2-HA protein and human anti-ACE2 antibodies was then evaluated by staining with goat anti-mouse Alexa Fluor™ 594 and or goat anti-human Alexa Fluor™ 488 antibodies (ThermoFisher Scientific). Images were captured using an DeltaVision OMX SR imaging system (GE Healthcare). Internalization staining of hACE2 antibodies are shown in FIG. 6B (2G7A1), FIG. 6C (05B04), and FIG. 6D (05H02). A control is shown in FIG. 6E where no human ACE2 antibody was used. The results show how the binding of human anti-ACE2 antibodies do not cause internalization of ACE2.

### Example 9. Human ACE2 Antibodies Pharmacokinetics in Mice

Six week old hACE2-knock-in female mice, in which human ACE2 cDNA replaces the endogenous mouse ACE2 sequences, were obtained from Jackson Labs (B6.129S2(Cg)-Ace2tml(ACE2)Dwnt/J, strain 035000). After acclimatization for 2 weeks, these mice received subcutaneous injections of 250 g of human anti-ACE2 antibodies per mouse (n=5). Mice were bled on day 0, day 1, day 3, day 7, and day 14 with blood collected into Microvette® CB 300 Serum (Sarstedt).

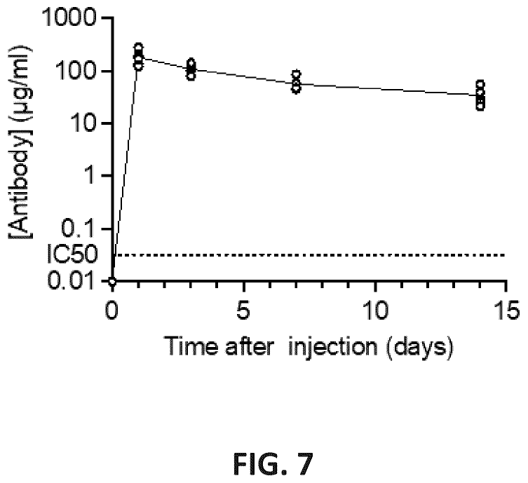

Serially diluted mouse plasma (five-fold serial dilution over four dilutions from a maximum volume of 0.5 μl) was diluted in PBS buffer containing 2% BSA and mixed with 30 ng of His-tagged ACE2(1-740aa)-NanoLuc protein. After 1 hour incubation at 4° C., the mixture was incubated with 3 μl of Dynabeads™ Protein G magnetic beads (ThermoFisher Scientific). After 1 hour rotation at 4° C., the beads were washed three times and bound NanoLuc activity measured using Nano-Glo Luciferase Assay System and a GloMax® Navigator Microplate Luminometer (Promega). In order to construct standard calibration curves for measurement of antibody levels in plasma, 100 ng of antibodies (2G7A1, 05B04, 05H02, or 05B04LC/05D06HC) were five-fold serially over seven dilutions were and mixed with ACE2(1-740aa)-NanoLuc proteins. Antibody: ACE2(1-740aa)-NanoLuc complexes were captured and quantified in parallel with those formed using the mouse plasma samples. FIG. 7 shows the plasma antibody concentrations in mice following subcutaneous injection of 250 g of 05B04 antibody on day 0. The dashed line represents the IC50 for inhibition of SARS-CoV-2 pseudovirus infection.

### Example 10. Protection of hACE2 Knock-In Mice from SARS-CoV-2 Infection

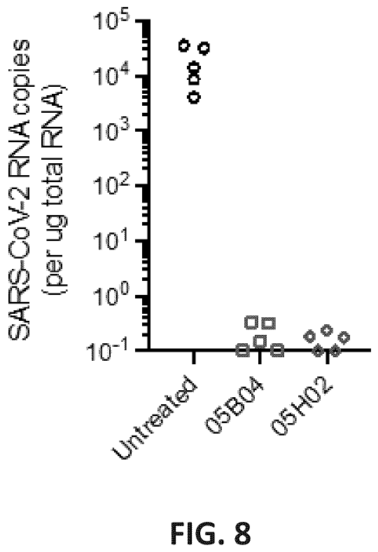

Six week old hACE2-knock-in female mice, in which human ACE2 cDNA replaces the endogenous mouse ACE2 sequences, were obtained from Jackson Labs (B6.129S2(Cg)-Ace2tml(ACE2)Dwnt/J, strain 035000). After acclimatization for 2 weeks, the mice (five mice per treatment group) were injected retro-orbitally with 250 g (equivalent to ˜12.5 mg/kg) of anti-human ACE2 antibodies. At 2 days after antibody injection, mice were challenged intranasally with SARS-CoV-2, USA_WA/2020 P3, 1×105 PFU/mouse (virus titers determined on VeroE6 cells). At 3 days after infection, mouse lungs were dissected and homogenized in TRIzol. Chloroform was added to induce phase separation. After centrifugation, RNA in the aqueous phase was precipitated with isopropanol and, after wash with ice-cold 75% ethanol, dissolved in nuclease-free water. The number of viral genomes per microgram of total lung RNA was measured by qRT-PCR, using Power SYBR Green RNA-to-CT 1-Step Kit (ThermoFisher Scientific) on StepOne Plus Real-Time PCR system (Applied Biosystems). The primers used target RNA sequences encoding the nucleocapsid protein: 2019-nCoV_N1-F: 5′-GACCCCAAAATCAGCGAAAT-3′ (SEQ ID NO: 95) and 2019-nCoV_N1-R: 5′-TCTGGTTACTGCCAGTTGAATCTG-3′ (SEQ ID NO: 96). The standard was obtained from Integrated DNA technologies (2019-nCoV_N_Positive Control 10006625). FIG. 8 shows how anti-ACE2 antibodies 05B04 and 05H02 protected hACE2 knock-in mice from SARS-CoV-2 infection.

TABLE 2
Sequence description

| SEQ ID | | |
|---|---|---|
| NO | Type | Description |
| 1 | Amino Acid | Human ACE2 (1-740aa)-Fc |
| 2 | Amino Acid | Human ACE2 (1-740aa)-8xHis |
| 3 | Nucleic Acid | 05B04 - Light chain |
| 4 | Amino Acid | 05B04 - Light chain |
| 5 | Nucleic Acid | 05B04 - Heavy chain |
| 6 | Amino Acid | 05B04 - Heavy chain |
| 7 | Nucleic Acid | 05D06 - Light chain |
| 8 | Amino Acid | 05D06 - Light chain |
| 9 | Nucleic Acid | 05D06 - Heavy chain |
| 10 | Amino Acid | 05D06 - Heavy chain |
| 11 | Nucleic Acid | 05E10 - Light chain |
| 12 | Amino Acid | 05E10- Light chain |
| 13 | Nucleic Acid | 05E10- Heavy chain |
| 14 | Amino Acid | 05E10- Heavy chain |
| 15 | Nucleic Acid | 05G01 - Light chain |
| 16 | Amino Acid | 05G01- Light chain |
| 17 | Nucleic Acid | 05G01- Heavy chain |
| 18 | Amino Acid | 05G01- Heavy chain |
| 19 | Nucleic Acid | 05H02 - Light chain |
| 20 | Amino Acid | 05H02- Light chain |
| 21 | Nucleic Acid | 05H02- Heavy chain |
| 22 | Amino Acid | 05H02- Heavy chain |
| 23 | Nucleic Acid | 2G7A1 - Light chain |
| 24 | Amino Acid | 2G7A1 - Light chain |
| 25 | Nucleic Acid | 2G7A1 - Heavy chain |
| 26 | Amino Acid | 2G7A1 - Heavy chain |
| 27 | Nucleic Acid | 2F6A6 - Light chain |
| 28 | Amino Acid | 2F6A6 - Light chain |
| 29 | Nucleic Acid | 2F6A6 - Heavy chain |
| 30 | Amino Acid | 2F6A6 - Heavy chain |
| 31 | Nucleic Acid | 4A12A4 - Light chain |
| 32 | Amino Acid | 4A12A4 - Light chain |
| 33 | Nucleic Acid | 4A12A4 - Heavy chain |
| 34 | Amino Acid | 4A12A4 - Heavy chain |
| 35 | Nucleic Acid | 2C12H3 - Light chain |
| 36 | Amino Acid | 2C12H3 - Light chain |
| 37 | Nucleic Acid | 2C12H3 - Heavy chain |
| 38 | Amino Acid | 2C12H3 - Heavy chain |
| 39 | Nucleic Acid | 1C9H1 - Light chain |
| 40 | Amino Acid | 1C9H1 - Light chain |
| 41 | Nucleic Acid | 1C9H1 - Heavy chain |
| 42 | Amino Acid | 1C9H1 - Heavy chain |
| 43 | Amino Acid | 05B04 - Heavy chain CDR1 |
| 44 | Amino Acid | 05B04 - Heavy chain CDR2 |
| 45 | Amino Acid | 05B04 - Heavy chain CDR3 |
| 46 | Amino Acid | 05B04 - Light chain CDR1 |
| 47 | Amino Acid | 05B04 - Light chain CDR3 |
| 48 | Amino Acid | 05D06 - Heavy chain CDR1 |
| 49 | Amino Acid | 05D06 - Heavy chain CDR2 |
| 50 | Amino Acid | 05D06 - Heavy chain CDR3 |
| 51 | Amino Acid | 05D06 - Light chain CDR1 |
| 52 | Amino Acid | 05D06 - Light chain CDR3 |
| 53 | Amino Acid | 05E10 - Heavy chain CDR1 |
| 54 | Amino Acid | 05E10 - Heavy chain CDR2 |
| 55 | Amino Acid | 05E10 - Heavy chain CDR3 |
| 56 | Amino Acid | 05E10 - Light chain CDR1 |
| 57 | Amino Acid | 05E10 - Light chain CDR3 |
| 58 | Amino Acid | 05G01 - Heavy chain CDR1 |
| 59 | Amino Acid | 05G01 - Heavy chain CDR2 |
| 60 | Amino Acid | 05G01 - Heavy chain CDR3 |
| 61 | Amino Acid | 05G01 - Light chain CDR1 |
| 62 | Amino Acid | 05G01 - Light chain CDR3 |
| 63 | Amino Acid | 05H02 - Heavy chain CDR1 |
| 64 | Amino Acid | 05H02 - Heavy chain CDR2 |
| 65 | Amino Acid | 05H02 - Heavy chain CDR3 |
| 66 | Amino Acid | 05H02 - Light chain CDR1 |
| 67 | Amino Acid | 05H02 - Light chain CDR3 |
| 68 | Amino Acid | 2G7A1 - Heavy chain CDR1 |
| 69 | Amino Acid | 2G7A1 - Heavy chain CDR2 |
| 70 | Amino Acid | 2G7A1 - Heavy chain CDR3 |
| 71 | Amino Acid | 2G7A1 - Light chain CDR1 |
| 72 | Amino Acid | 2G7A1 - Light chain CDR3 |
| 73 | Amino Acid | 2F6A6 - Heavy chain CDR1 |
| 74 | Amino Acid | 2F6A6 - Heavy chain CDR2 |
| 75 | Amino Acid | 2F6A6 - Heavy chain CDR3 |
| 76 | Amino Acid | 2F6A6 - Light chain CDR1 |
| 77 | Amino Acid | 2F6A6 - Light chain CDR3 |
| 78 | Amino Acid | 4A12A4 - Heavy chain CDR1 |
| 79 | Amino Acid | 4A12A4 - Heavy chain CDR2 |
| 80 | Amino Acid | 4A12A4 - Heavy chain CDR3 |
| 81 | Amino Acid | 4A12A4 - Light chain CDR1 |
| 82 | Amino Acid | 4A12A4 - Light chain CDR3 |
| 83 | Amino Acid | 2C12H3 - Heavy chain CDR1 |
| 84 | Amino Acid | 2C12H3 - Heavy chain CDR2 |
| 85 | Amino Acid | 2C12H3 - Heavy chain CDR3 |
| 86 | Amino Acid | 2C12H3 - Light chain CDR1 |
| 87 | Amino Acid | 2C12H3 - Light chain CDR3 |
| 88 | Amino Acid | 1C9H1 - Heavy chain CDR1 |
| 89 | Amino Acid | 1C9H1 - Heavy chain CDR2 |
| 90 | Amino Acid | 1C9H1 - Heavy chain CDR3 |
| 91 | Amino Acid | 1C9H1 - Light chain CDR1 |
| 92 | Amino Acid | 1C9H1 - Light chain CDR3 |
| 93 | Nucleic Acid | Human ACE2 |
| 94 | Amino Acid | Human ACE2 isoform 1 |
| 95 | Nucleic Acid | Primer 2019-nCoV_N1 Forward |
| 96 | Nucleic Acid | Primer 2019-nCoV_N1 Reverse |

1. An isolated antibody or antigen binding fragment thereof, wherein the antibody or antigen binding fragment thereof
(i) binds to the extracellular domain of a human angiotensin converting enzyme 2 (ACE2) protein, wherein binding of the human ACE2 protein by the antibody or antigen binding fragment thereof does not interfere with the enzymatic activity of the human ACE2 protein and does not induce internalization of the human ACE2 protein; and
(ii) binds to the same epitope on the extracellular domain of the human ACE2 protein as a reference antibody or antigen-binding fragment thereof, or competes for binding to the extracellular domain of the human ACE2 protein with the reference antibody or antigen-binding fragment thereof, wherein the reference antibody or antigen-binding fragment thereof comprises:
a) a heavy chain variable domain selected from the group consisting of:
a heavy chain variable domain comprising a complementarity determining region (CDR)1-H comprising the amino acid sequence GFTFSSYA (SEQ ID NO: 43) or a variant thereof, a CDR2-H comprising the amino acid sequence ISGSGDRT (SEQ ID NO: 44) or a variant thereof, and a CDR3-H comprising the amino acid sequence AKDWAMVGADAFDI (SEQ ID NO: 45) or a variant thereof;
a heavy chain variable domain comprising a CDR1-H comprising the amino acid sequence GFTFSSYA (SEQ ID NO: 48) or a variant thereof; a CDR2-H comprising the amino acid sequence ISISGGST (SEQ ID NO: 49) or a variant thereof; and a CDR3-H comprising the amino acid sequence VKDWYIVGADAFDI (SEQ ID NO: 50) or a variant thereof;
a heavy chain variable domain comprising a CDR1-H comprising then amino acid sequence GFTFSSYA (SEQ ID NO: 53) or a variant thereof; a CDR2-H comprising the amino acid sequence INISGGST (SEQ ID NO: 54) or a variant thereof; and a CDR3-H comprising the amino acid sequence VKDWYIMGADAFDI (SEQ ID NO: 55) or a variant thereof;
a heavy chain variable domain comprising a CDR1-H comprising the amino acid sequence GFTFSNYA (SEQ ID NO: 58) or a variant thereof; a CDR2-H comprising the amino acid sequence ISINGDRT (SEQ ID NO: 59) or a variant thereof; and a CDR3-H comprising the amino acid sequence AKDWAIVGADAFDV (SEQ ID NO: 60) or a variant thereof;
a heavy chain variable domain comprising a CDR1-H comprising the amino acid sequence GFTFNIYA (SEQ ID NO: 63) or a variant thereof; a CDR2-H comprising the amino acid sequence FSGSRYNT (SEQ ID NO: 64) or a variant thereof; and a CDR3-H comprising the amino acid sequence of AKEAVAGQFDY (SEQ ID NO: 65) or a variant thereof;
a heavy chain variable domain comprising a CDR1-H comprising the amino acid sequence DGSISGYY (SEQ ID NO: 68) or a variant thereof; a CDR2-H comprising the amino acid sequence IHYSGTT (SEQ ID NO: 69) or a variant thereof; and a CDR3-H comprising the amino acid sequence ARGPVFWYFDL (SEQ ID NO: 70) or a variant thereof;
a heavy chain variable domain comprising a CDR1-H comprising the amino acid sequence DGSISGYY (SEQ ID NO: 73) or a variant thereof; a CDR2-H comprising then amino acid sequence IYYSGTT (SEQ ID NO: 74) or a variant thereof; and a CDR3-H comprising the amino acid sequence ARGPVFWYFDL (SEQ ID NO: 75) or a variant thereof;
a heavy chain variable domain comprising a CDR1-H comprising the amino acid sequence GFTFSSYS (SEQ ID NO: 78) or a variant thereof; a CDR2-H comprising the amino acid sequence ITNGINNI (SEQ ID NO: 79) or a variant thereof; and a CDR3-H comprising the amino acid sequence ARYYYYYGMDV (SEQ ID NO: 80) or a variant thereof;
a heavy chain variable domain comprising a CDR1-H comprising the amino acid sequence GGSITSYY (SEQ ID NO: 83) or a variant thereof; a CDR2-H comprising the amino acid sequence IFSSGIT (SEQ ID NO: 84) or a variant thereof; and a CDR3-H comprising the amino acid sequence ARHDGFGWFDP (SEQ ID NO: 85) or a variant thereof;
a heavy chain variable domain comprising a CDR1-H comprising the amino acid sequence GFSLSTPGVA (SEQ ID NO: 88) or a variant thereof; a CDR2-H comprising the amino acid sequence IFWNDDE (SEQ ID NO: 89) or a variant thereof; and a CDR3-H comprising the amino acid sequence AHLALYWYFDF (SEQ ID NO: 90) or a variant thereof;
and/or
b) a light chain variable domain selected from the group consisting of:
a light chain variable domain comprising a CDR1-L comprising the amino acid sequence QGISNY (SEQ ID NO: 46) or a variant thereof; a CDR2-L comprising the amino acid sequence AAS or a variant thereof; and a CDR3-L comprising the amino acid sequence LQHSYYPYT (SEQ ID NO: 47) or a variant thereof;
a light chain variable domain comprising a CDR1-L comprising the amino acid sequence QGISNY (SEQ ID NO: 51) or a variant thereof; a CDR2-L comprising the amino acid sequence ATS or a variant thereof; and a CDR3-L comprising the amino acid sequence LQHSNYPYT (SEQ ID NO: 52) or a variant thereof;
a light chain variable domain comprising a CDR1-L comprising the amino acid sequence QGISNY (SEQ ID NO: 56) or a variant thereof; a CDR2-L comprising the amino acid sequence ATS or a variant thereof; and a CDR3-L comprising the amino acid sequence LQHSNYPYT (SEQ ID NO: 57) or a variant thereof;
a light chain variable domain comprising a CDR1-L comprising the amino acid sequence QVISNY (SEQ ID NO: 61) or a variant thereof; a CDR2-L comprising the amino acid sequence AGS or a variant thereof; and a CDR3-L comprising the amino acid sequence LQHNNYPYT (SEQ ID NO: 62) or a variant thereof;
a light chain variable domain comprising a CDR1-L comprising the amino acid sequence QSISSW (SEQ ID NO: 66) or a variant thereof; a CDR2-L comprising the amino acid sequence KAS or a variant thereof; and a CDR3-L comprising the amino acid sequence QQYNTYSRT (SEQ ID NO: 67) or a variant thereof;
a light chain variable domain comprising a CDR1-L comprising the amino acid sequence SGSINNNY (SEQ ID NO: 71) or a variant thereof; a CDR2-L comprising the amino acid sequence DDN or a variant thereof; and a CDR3-L comprising the amino acid sequence QSYDSSSRV (SEQ ID NO: 72) or a variant thereof;
a light chain variable domain comprising a CDR1-L comprising the amino acid sequence SGSISNSY (SEQ ID NO: 76) or a variant thereof; a CDR2-L comprising the amino acid sequence DDN or a variant thereof; and a CDR3-L comprising the amino acid sequence QSYDSSSRV (SEQ ID NO: 77) or a variant thereof;
a light chain variable domain comprising a CDR1-L comprising the amino acid sequence SLRNYY (SEQ ID NO: 81) or a variant thereof; a CDR2-L comprising the amino acid sequence GKN or a variant thereof; and a CDR3-L comprising the amino acid sequence NSRDSGGNHVV (SEQ ID NO: 82);
a light chain variable domain comprising a CDR1-L comprising the amino acid sequence QSVSSSY (SEQ ID NO: 86) or a variant thereof; a CDR2-L comprising the amino acid sequence GAS or a variant thereof; and a CDR3-L comprising the amino acid sequence QQYGSSPRT (SEQ ID NO: 87) or a variant thereof; and
a light chain variable domain comprising a CDR1-L comprising the amino acid sequence SSDVGSYNL (SEQ ID NO: 91) or a variant thereof; a CDR2-L comprising the amino acid sequence EGS or a variant thereof; and a CDR3-L comprising the amino acid sequence CSYAGSNIVV (SEQ ID NO: 92) or a variant thereof.

2. An isolated antibody or antigen binding fragment thereof, wherein the antibody or antigen binding fragment thereof comprises:
a) a heavy chain variable domain selected from the group consisting of:
a heavy chain variable domain comprising a complementarity determining region (CDR)1-H comprising the amino acid sequence GFTFSSYA (SEQ ID NO: 43) or a variant thereof, a CDR2-H comprising the amino acid sequence ISGSGDRT (SEQ ID NO: 44) or a variant thereof, and a CDR3-H comprising the amino acid sequence AKDWAMVGADAFDI (SEQ ID NO: 45) or a variant thereof;
a heavy chain variable domain comprising a CDR1-H comprising the amino acid sequence GFTFSSYA (SEQ ID NO: 48) or a variant thereof; a CDR2-H comprising the amino acid sequence ISISGGST (SEQ ID NO: 49) or a variant thereof; and a CDR3-H comprising the amino acid sequence VKDWYIVGADAFDI (SEQ ID NO: 50) or a variant thereof;
a heavy chain variable domain comprising a CDR1-H comprising then amino acid sequence GFTFSSYA (SEQ ID NO: 53) or a variant thereof; a CDR2-H comprising the amino acid sequence INISGGST (SEQ ID NO: 54) or a variant thereof; and a CDR3-H comprising the amino acid sequence VKDWYIMGADAFDI (SEQ ID NO: 55) or a variant thereof;
a heavy chain variable domain comprising a CDR1-H comprising the amino acid sequence GFTFSNYA (SEQ ID NO: 58) or a variant thereof; a CDR2-H comprising the amino acid sequence ISINGDRT (SEQ ID NO: 59) or a variant thereof; and a CDR3-H comprising the amino acid sequence AKDWAIVGADAFDV (SEQ ID NO: 60) or a variant thereof;
a heavy chain variable domain comprising a CDR1-H comprising the amino acid sequence GFTFNIYA (SEQ ID NO: 63) or a variant thereof; a CDR2-H comprising the amino acid sequence FSGSRYNT (SEQ ID NO: 64) or a variant thereof; and a CDR3-H comprising the amino acid sequence of AKEAVAGQFDY (SEQ ID NO: 65) or a variant thereof;
a heavy chain variable domain comprising a CDR1-H comprising the amino acid sequence DGSISGYY (SEQ ID NO: 68) or a variant thereof; a CDR2-H comprising the amino acid sequence IHYSGTT (SEQ ID NO: 69) or a variant thereof; and a CDR3-H comprising the amino acid sequence ARGPVFWYFDL (SEQ ID NO: 70) or a variant thereof;
a heavy chain variable domain comprising a CDR1-H comprising the amino acid sequence DGSISGYY (SEQ ID NO: 73) or a variant thereof; a CDR2-H comprising then amino acid sequence IYYSGTT (SEQ ID NO: 74) or a variant thereof; and a CDR3-H comprising the amino acid sequence ARGPVFWYFDL (SEQ ID NO: 75) or a variant thereof;
a heavy chain variable domain comprising a CDR1-H comprising the amino acid sequence GFTFSSYS (SEQ ID NO: 78) or a variant thereof; a CDR2-H comprising the amino acid sequence ITNGINNI (SEQ ID NO: 79) or a variant thereof; and a CDR3-H comprising the amino acid sequence ARYYYYYGMDV (SEQ ID NO: 80) or a variant thereof;
a heavy chain variable domain comprising a CDR1-H comprising the amino acid sequence GGSITSYY (SEQ ID NO: 83) or a variant thereof; a CDR2-H comprising the amino acid sequence IFSSGIT (SEQ ID NO: 84) or a variant thereof; and a CDR3-H comprising the amino acid sequence ARHDGFGWFDP (SEQ ID NO: 85) or a variant thereof;
a heavy chain variable domain comprising a CDR1-H comprising the amino acid sequence GFSLSTPGVA (SEQ ID NO: 88) or a variant thereof; a CDR2-H comprising the amino acid sequence IFWNDDE (SEQ ID NO: 89) or a variant thereof; and a CDR3-H comprising the amino acid sequence AHLALYWYFDF (SEQ ID NO: 90) or a variant thereof;
and/or
b) a light chain variable domain selected from the group consisting of:
a light chain variable domain comprising a CDR1-L comprising the amino acid sequence QGISNY (SEQ ID NO: 46) or a variant thereof; a CDR2-L comprising the amino acid sequence AAS or a variant thereof; and a CDR3-L comprising the amino acid sequence LQHSYYPYT (SEQ ID NO: 47) or a variant thereof;
a light chain variable domain comprising a CDR1-L comprising the amino acid sequence QGISNY (SEQ ID NO: 51) or a variant thereof; a CDR2-L comprising the amino acid sequence ATS or a variant thereof; and a CDR3-L comprising the amino acid sequence LQHSNYPYT (SEQ ID NO: 52) or a variant thereof;
a light chain variable domain comprising a CDR1-L comprising the amino acid sequence QGISNY (SEQ ID NO: 56) or a variant thereof; a CDR2-L comprising the amino acid sequence ATS or a variant thereof; and a CDR3-L comprising the amino acid sequence LQHSNYPYT (SEQ ID NO: 57) or a variant thereof;
a light chain variable domain comprising a CDR1-L comprising the amino acid sequence QVISNY (SEQ ID NO: 61) or a variant thereof; a CDR2-L comprising the amino acid sequence AGS or a variant thereof; and a CDR3-L comprising the amino acid sequence LQHNNYPYT (SEQ ID NO: 62) or a variant thereof;
a light chain variable domain comprising a CDR1-L comprising the amino acid sequence QSISSW (SEQ ID NO: 66) or a variant thereof; a CDR2-L comprising the amino acid sequence KAS or a variant thereof; and a CDR3-L comprising the amino acid sequence QQYNTYSRT (SEQ ID NO: 67) or a variant thereof;
a light chain variable domain comprising a CDR1-L comprising the amino acid sequence SGSINNNY (SEQ ID NO: 71) or a variant thereof; a CDR2-L comprising the amino acid sequence DDN or a variant thereof; and a CDR3-L comprising the amino acid sequence QSYDSSSRV (SEQ ID NO: 72) or a variant thereof;
a light chain variable domain comprising a CDR1-L comprising the amino acid sequence SGSISNSY (SEQ ID NO: 76) or a variant thereof; a CDR2-L comprising the amino acid sequence DDN or a variant thereof; and a CDR3-L comprising the amino acid sequence QSYDSSSRV (SEQ ID NO: 77) or a variant thereof;
a light chain variable domain comprising a CDR1-L comprising the amino acid sequence SLRNYY (SEQ ID NO: 81) or a variant thereof; a CDR2-L comprising the amino acid sequence GKN or a variant thereof; and a CDR3-L comprising the amino acid sequence NSRDSGGNHVV (SEQ ID NO: 82);
a light chain variable domain comprising a CDR1-L comprising the amino acid sequence QSVSSSY (SEQ ID NO: 86) or a variant thereof; a CDR2-L comprising the amino acid sequence GAS or a variant thereof; and a CDR3-L comprising the amino acid sequence QQYGSSPRT (SEQ ID NO: 87) or a variant thereof; and
a light chain variable domain comprising a CDR1-L comprising the amino acid sequence SSDVGSYNL (SEQ ID NO: 91) or a variant thereof; a CDR2-L comprising the amino acid sequence EGS or a variant thereof; and a CDR3-L comprising the amino acid sequence CSYAGSNIVV (SEQ ID NO: 92) or a variant thereof.

3. The isolated antibody or antigen binding fragment thereof of claim 1, wherein the antibody or antigen binding fragment thereof comprises:
a heavy chain variable domain comprising an amino acid sequence having at least 90% sequence identity to the amino acid sequence as set forth in SEQ ID NOS: 6, 10, 14, 18, 22, 26, 30, 34, 38, or 42; and/or
a light chain variable domain comprising an amino acid sequence having at least 90% sequence identity to the amino acid sequence as set forth in SEQ ID NOS: 4, 8, 12, 16, 20, 24, 28, 32, 36, or 40.

4. The isolated antibody or antigen binding fragment thereof of claim 1, wherein the antibody or antigen binding fragment thereof comprises:
a heavy chain variable domain comprising
a (CDR)1-H comprising the amino acid sequence GFTFSSYA (SEQ ID NO: 43) or a variant thereof, a CDR2-H comprising the amino acid sequence ISGSGDRT (SEQ ID NO: 44) or a variant thereof, and a CDR3-H comprising the amino acid sequence AKDWAMVGADAFDI (SEQ ID NO: 45) or a variant thereof; and/or
a CDR1-H comprising the amino acid sequence GFTFSSYA (SEQ ID NO: 48) or a variant thereof; a CDR2-H comprising the amino acid sequence ISISGGST (SEQ ID NO: 49) or a variant thereof; and a CDR3-H comprising the amino acid sequence VKDWYIVGADAFDI (SEQ ID NO: 50) or a variant thereof; and/or
a CDR1-H comprising then amino acid sequence GFTFSSYA (SEQ ID NO: 53) or a variant thereof; a CDR2-H comprising the amino acid sequence INISGGST (SEQ ID NO: 54) or a variant thereof; and a CDR3-H comprising the amino acid sequence VKDWYIMGADAFDI (SEQ ID NO: 55) or a variant thereof; and/or
a CDR1-H comprising the amino acid sequence GFTFSNYA (SEQ ID NO: 58) or a variant thereof; a CDR2-H comprising the amino acid sequence ISINGDRT (SEQ ID NO: 59) or a variant thereof; and a CDR3-H comprising the amino acid sequence AKDWAIVGADAFDV (SEQ ID NO: 60) or a variant thereof; and/or
a CDR1-H comprising the amino acid sequence GFTFNIYA (SEQ ID NO: 63) or a variant thereof; a CDR2-H comprising the amino acid sequence FSGSRYNT (SEQ ID NO: 64) or a variant thereof; and a CDR3-H comprising the amino acid sequence of AKEAVAGQFDY (SEQ ID NO: 65) or a variant thereof; and/or
a CDR1-H comprising the amino acid sequence DGSISGYY (SEQ ID NO: 68) or a variant thereof; a CDR2-H comprising the amino acid sequence IHYSGTT (SEQ ID NO: 69) or a variant thereof; and a CDR3-H comprising the amino acid sequence ARGPVFWYFDL (SEQ ID NO: 70) or a variant thereof; and/or
a CDR1-H comprising the amino acid sequence DGSISGYY (SEQ ID NO: 73) or a variant thereof; a CDR2-H comprising then amino acid sequence IYYSGTT (SEQ ID NO: 74) or a variant thereof; and a CDR3-H comprising the amino acid sequence ARGPVFWYFDL (SEQ ID NO: 75) or a variant thereof; and/or
a CDR1-H comprising the amino acid sequence GFTFSSYS (SEQ ID NO: 78) or a variant thereof; a CDR2-H comprising the amino acid sequence ITNGINNI (SEQ ID NO: 79) or a variant thereof; and a CDR3-H comprising the amino acid sequence ARYYYYYGMDV (SEQ ID NO: 80) or a variant thereof; and/or
a CDR1-H comprising the amino acid sequence GGSITSYY (SEQ ID NO: 83) or a variant thereof; a CDR2-H comprising the amino acid sequence IFSSGIT (SEQ ID NO: 84) or a variant thereof; and a CDR3-H comprising the amino acid sequence ARHDGFGWFDP (SEQ ID NO: 85) or a variant thereof; and/or
a CDR1-H comprising the amino acid sequence GFSLSTPGVA (SEQ ID NO: 88) or a variant thereof; a CDR2-H comprising the amino acid sequence IFWNDDE (SEQ ID NO: 89) or a variant thereof; and a CDR3-H comprising the amino acid sequence AHLALYWYFDF (SEQ ID NO: 90) or a variant thereof;
and/or
a light chain variable domain comprising:
a CDR1-L comprising the amino acid sequence QGISNY (SEQ ID NO: 46) or a variant thereof; a CDR2-L comprising the amino acid sequence AAS or a variant thereof; and a CDR3-L comprising the amino acid sequence LQHSYYPYT (SEQ ID NO: 47) or a variant thereof; and/or
a CDR1-L comprising the amino acid sequence QGISNY (SEQ ID NO: 51) or a variant thereof; a CDR2-L comprising the amino acid sequence ATS or a variant thereof; and a CDR3-L comprising the amino acid sequence LQHSNYPYT (SEQ ID NO: 52) or a variant thereof; and/or
a CDR1-L comprising the amino acid sequence QGISNY (SEQ ID NO: 56) or a variant thereof; a CDR2-L comprising the amino acid sequence ATS or a variant thereof; and a CDR3-L comprising the amino acid sequence LQHSNYPYT (SEQ ID NO: 57) or a variant thereof; and/or
a CDR1-L comprising the amino acid sequence QVISNY (SEQ ID NO: 61) or a variant thereof; a CDR2-L comprising the amino acid sequence AGS or a variant thereof; and a CDR3-L comprising the amino acid sequence LQHNNYPYT (SEQ ID NO: 62) or a variant thereof; and/or
a CDR1-L comprising the amino acid sequence QSISSW (SEQ ID NO: 66) or a variant thereof; a CDR2-L comprising the amino acid sequence KAS or a variant thereof; and a CDR3-L comprising the amino acid sequence QQYNTYSRT (SEQ ID NO: 67) or a variant thereof; and/or
a CDR1-L comprising the amino acid sequence SGSINNNY (SEQ ID NO: 71) or a variant thereof; a CDR2-L comprising the amino acid sequence DDN or a variant thereof; and a CDR3-L comprising the amino acid sequence QSYDSSSRV (SEQ ID NO: 72) or a variant thereof; and/or
a CDR1-L comprising the amino acid sequence SGSISNSY (SEQ ID NO: 76) or a variant thereof; a CDR2-L comprising the amino acid sequence DDN or a variant thereof; and a CDR3-L comprising the amino acid sequence QSYDSSSRV (SEQ ID NO: 77) or a variant thereof; and/or
a CDR1-L comprising the amino acid sequence SLRNYY (SEQ ID NO: 81) or a variant thereof; a CDR2-L comprising the amino acid sequence GKN or a variant thereof; and a CDR3-L comprising the amino acid sequence NSRDSGGNHVV (SEQ ID NO: 82); and/or
a CDR1-L comprising the amino acid sequence QSVSSSY (SEQ ID NO: 86) or a variant thereof; a CDR2-L comprising the amino acid sequence GAS or a variant thereof; and a CDR3-L comprising the amino acid sequence QQYGSSPRT (SEQ ID NO: 87) or a variant thereof; and/or
a CDR1-L comprising the amino acid sequence SSDVGSYNL (SEQ ID NO: 91) or a variant thereof; a CDR2-L comprising the amino acid sequence EGS or a variant thereof; and a CDR3-L comprising the amino acid sequence CSYAGSNIVV (SEQ ID NO: 92) or a variant thereof.

5. The isolated antibody or antigen-binding fragment thereof according to claim 1, comprising:
(a) a heavy chain variable domain comprising a complementarity determining region (CDR)1-H comprising the amino acid sequence GFTFSSYA (SEQ ID NO: 43) or a variant thereof, a CDR2-H comprising the amino acid sequence ISGSGDRT (SEQ ID NO: 44) or a variant thereof, and a CDR3-H comprising the amino acid sequence AKDWAMVGADAFDI (SEQ ID NO: 45) or a variant thereof; and
a light chain variable domain comprising a CDR1-L comprising the amino acid sequence QGISNY (SEQ ID NO: 46) or a variant thereof; a CDR2-L comprising the amino acid sequence AAS or a variant thereof; and a CDR3-L comprising the amino acid sequence LQHSYYPYT (SEQ ID NO: 47) or a variant thereof;
(b) a heavy chain variable domain comprising a CDR1-H comprising the amino acid sequence GFTFSSYA (SEQ ID NO: 48) or a variant thereof; a CDR2-H comprising the amino acid sequence ISISGGST (SEQ ID NO: 49) or a variant thereof; and a CDR3-H comprising the amino acid sequence VKDWYIVGADAFDI (SEQ ID NO: 50) or a variant thereof; and
a light chain variable domain comprising a CDR1-L comprising the amino acid sequence QGISNY (SEQ ID NO: 51) or a variant thereof; a CDR2-L comprising the amino acid sequence ATS or a variant thereof; and a CDR3-L comprising the amino acid sequence LQHSNYPYT (SEQ ID NO: 52) or a variant thereof;
(c) a heavy chain variable domain comprising a CDR1-H comprising then amino acid sequence GFTFSSYA (SEQ ID NO: 53) or a variant thereof; a CDR2-H comprising the amino acid sequence INISGGST (SEQ ID NO: 54) or a variant thereof; and a CDR3-H comprising the amino acid sequence VKDWYIMGADAFDI (SEQ ID NO: 55) or a variant thereof; and
a light chain variable domain comprising a CDR1-L comprising the amino acid sequence QGISNY (SEQ ID NO: 56) or a variant thereof; a CDR2-L comprising the amino acid sequence ATS or a variant thereof; and a CDR3-L comprising the amino acid sequence LQHSNYPYT (SEQ ID NO: 57) or a variant thereof;
(d) a heavy chain variable domain comprising a CDR1-H comprising the amino acid sequence GFTFSNYA (SEQ ID NO: 58) or a variant thereof; a CDR2-H comprising the amino acid sequence ISINGDRT (SEQ ID NO: 59) or a variant thereof; and a CDR3-H comprising the amino acid sequence AKDWAIVGADAFDV (SEQ ID NO: 60) or a variant thereof; and
a light chain variable domain comprising a CDR1-L comprising the amino acid sequence QVISNY (SEQ ID NO: 61) or a variant thereof; a CDR2-L comprising the amino acid sequence AGS or a variant thereof; and a CDR3-L comprising the amino acid sequence LQHNNYPYT (SEQ ID NO: 62) or a variant thereof;
(e) a heavy chain variable domain comprising a CDR1-H comprising the amino acid sequence GFTFNIYA (SEQ ID NO: 63) or a variant thereof; a CDR2-H comprising the amino acid sequence FSGSRYNT (SEQ ID NO: 64) or a variant thereof; and a CDR3-H comprising the amino acid sequence of AKEAVAGQFDY (SEQ ID NO: 65) or a variant thereof; and
a light chain variable domain comprising a CDR1-L comprising the amino acid sequence QSISSW (SEQ ID NO: 66) or a variant thereof; a CDR2-L comprising the amino acid sequence KAS or a variant thereof; and a CDR3-L comprising the amino acid sequence QQYNTYSRT (SEQ ID NO: 67) or a variant thereof;
(f) a heavy chain variable domain comprising a CDR1-H comprising the amino acid sequence DGSISGYY (SEQ ID NO: 68) or a variant thereof; a CDR2-H comprising the amino acid sequence IHYSGTT (SEQ ID NO: 69) or a variant thereof; and a CDR3-H comprising the amino acid sequence ARGPVFWYFDL (SEQ ID NO: 70) or a variant thereof; and
a light chain variable domain comprising a CDR1-L comprising the amino acid sequence SGSINNNY (SEQ ID NO: 71) or a variant thereof; a CDR2-L comprising the amino acid sequence DDN or a variant thereof; and a CDR3-L comprising the amino acid sequence QSYDSSSRV (SEQ ID NO: 72) or a variant thereof;
(g) a heavy chain variable domain comprising a CDR1-H comprising the amino acid sequence DGSISGYY (SEQ ID NO: 73) or a variant thereof; a CDR2-H comprising then amino acid sequence IYYSGTT (SEQ ID NO: 74) or a variant thereof; and a CDR3-H comprising the amino acid sequence ARGPVFWYFDL (SEQ ID NO: 75) or a variant thereof; and
a light chain variable domain comprising a CDR1-L comprising the amino acid sequence SGSISNSY (SEQ ID NO: 76) or a variant thereof; a CDR2-L comprising the amino acid sequence DDN or a variant thereof; and a CDR3-L comprising the amino acid sequence QSYDSSSRV (SEQ ID NO: 77) or a variant thereof;
(h) a heavy chain variable domain comprising a CDR1-H comprising the amino acid sequence GFTFSSYS (SEQ ID NO: 78) or a variant thereof; a CDR2-H comprising the amino acid sequence ITNGINNI (SEQ ID NO: 79) or a variant thereof; and a CDR3-H comprising the amino acid sequence ARYYYYYGMDV (SEQ ID NO: 80) or a variant thereof; and
a light chain variable domain comprising a CDR1-L comprising the amino acid sequence SLRNYY (SEQ ID NO: 81) or a variant thereof; a CDR2-L comprising the amino acid sequence GKN or a variant thereof; and a CDR3-L comprising the amino acid sequence NSRDSGGNHVV (SEQ ID NO: 82);
(i) a heavy chain variable domain comprising a CDR1-H comprising the amino acid sequence GGSITSYY (SEQ ID NO: 83) or a variant thereof; a CDR2-H comprising the amino acid sequence IFSSGIT (SEQ ID NO: 84) or a variant thereof; and a CDR3-H comprising the amino acid sequence ARHDGFGWFDP (SEQ ID NO: 85) or a variant thereof; and
a light chain variable domain comprising a CDR1-L comprising the amino acid sequence QSVSSSY (SEQ ID NO: 86) or a variant thereof; a CDR2-L comprising the amino acid sequence GAS or a variant thereof; and a CDR3-L comprising the amino acid sequence QQYGSSPRT (SEQ ID NO: 87) or a variant thereof; and
or
(j) a heavy chain variable domain comprising a CDR1-H comprising the amino acid sequence GFSLSTPGVA (SEQ ID NO: 88) or a variant thereof; a CDR2-H comprising the amino acid sequence IFWNDDE (SEQ ID NO: 89) or a variant thereof; and a CDR3-H comprising the amino acid sequence AHLALYWYFDF (SEQ ID NO: 90) or a variant thereof; and
a light chain variable domain comprising a CDR1-L comprising the amino acid sequence SSDVGSYNL (SEQ ID NO: 91) or a variant thereof; a CDR2-L comprising the amino acid sequence EGS or a variant thereof; and a CDR3-L comprising the amino acid sequence CSYAGSNIVV (SEQ ID NO: 92) or a variant thereof.

6. The isolated antibody or antigen-binding fragment thereof according to claim 1, comprising:
a heavy chain variable domain comprising the amino acid sequence as set forth in SEQ ID NOS: 6, 10, 14, 18, 22, 26, 30, 34, 38, or 42; and/or
a light chain variable domain comprising the amino acid sequence as set forth in SEQ ID NO: 4, 8, 12, 16, 20, 24, 28, 32, 36, or 40.

7. The isolated antibody or antigen-binding fragment thereof according to claim 1, comprising:
(a) a heavy chain variable domain comprising an amino acid sequence having at least 90% identity to the amino acid sequence as set forth in SEQ ID NO: 6, and a light chain variable domain comprising an amino acid sequence having at least 90% identity to the amino acid sequence as set forth in SEQ ID NO: 4;
(b) a heavy chain variable domain comprising an amino acid sequence having at least 90% identity to the amino acid sequence as set forth in SEQ ID NO: 10, and a light chain variable domain comprising an amino acid sequence having at least 90% identity to the amino acid sequence as set forth in SEQ ID NO: 8;
(c) a heavy chain variable domain comprising an amino acid sequence having at least 90% identity to the amino acid sequence as set forth in SEQ ID NO: 14, and a light chain variable domain comprising an amino acid sequence having at least 90% identity to the amino acid sequence as set forth in SEQ ID NO: 12;
(d) a heavy chain variable domain comprising an amino acid sequence having at least 90% identity to the amino acid sequence as set forth in SEQ ID NO: 18 and a light chain variable domain comprising an amino acid sequence having at least 90% identity to the amino acid sequence as set forth in SEQ ID NO: 16;
(e) a heavy chain variable domain comprising an amino acid sequence having at least 90% identity to the amino acid sequence as set forth in SEQ ID NO: 22, and a light chain variable domain comprising an amino acid sequence having at least 90% identity to the amino acid sequence as set forth in SEQ ID NO: 20;
(f) a heavy chain variable domain comprising an amino acid sequence having at least 90% identity to the amino acid sequence as set forth in SEQ ID NO: 26, and a light chain variable domain comprising an amino acid sequence having at least 90% identity to the amino acid sequence as set forth in SEQ ID NO: 24;
(g) a heavy chain variable domain comprising an amino acid sequence having at least 90% identity to the amino acid sequence as set forth in SEQ ID NO: 30, and a light chain variable domain comprising an amino acid sequence having at least 90% identity to the amino acid sequence as set forth in SEQ ID NO: 28;
(h) a heavy chain variable domain comprising an amino acid sequence having at least 90% identity to the amino acid sequence as set forth in SEQ ID NO: 34, and a light chain variable domain comprising an amino acid sequence having at least 90% identity to the amino acid sequence as set forth in SEQ ID NO: 32;
(i) a heavy chain variable domain comprising an amino acid sequence having at least 90% identity to the amino acid sequence as set forth in SEQ ID NO: 38, and a light chain variable domain comprising an amino acid sequence having at least 90% identity to the amino acid sequence as set forth in SEQ ID NO: 36; and/or
(j) a heavy chain variable domain comprising an amino acid sequence having at least 90% identity to the amino acid sequence as set forth in SEQ ID NO: 42, and a light chain variable domain comprising an amino acid sequence having at least 90% identity to the amino acid sequence as set forth in SEQ ID NO: 40.

8. The antibody or antigen-binding fragment thereof according to claim 1, wherein the heavy chain immunoglobulin variable domain is linked to an IgG, IgG1 or IgG4 heavy chain constant region and the light chain immunoglobulin variable region is linked to a human kappa or lambda light chain constant region.

9. The antibody or antigen-binding fragment thereof of claim 8, wherein the heavy chain immunoglobulin variable domain is linked to a human IgG1 constant region comprising substitutions at L234 L235 (LALA) that abolish FcR-gamma interaction, and/or substitutions at M428 N434 (LS) that enhance interaction with the neonatal Fc receptor to prolong antibody half-life in human.

10. The antibody or antigen-binding fragment thereof according to claim 1, wherein the antibody or antigen-binding fragment thereof is multispecific.

11. The antibody or antigen-binding fragment thereof according to claim 1, wherein the antibody or antigen-binding fragment thereof comprises one or more of the following additional properties:
(a) binds to the human ACE2 protein with a KD of about 10⁻⁹ M, 10⁻¹⁰ M, 10⁻¹¹ M, or 10⁻¹² M; e.g., about 7.66 nM;
(b) blocks binding of a coronavirus to the human ACE2 protein; and
(c) inhibits infection by a coronavirus.

12. The antibody or antigen-binding fragment thereof of claim 11, wherein the coronavirus is a SARS-CoV, a SARS-CoV-2, a Pangolin CoV, a Bat CoV, any other member of the betacoronavirus genus or sarbecovirus sub-genus that uses ACE2 to enter cells or any member of the alphacoronavirus genus.

13. The antibody or antigen-binding fragment thereof of claim 12, wherein the SARS-CoV-2 is a Wuhan-hu-1 variant, an Alpha variant, a Beta variant, a Delta variant, an Omicron variant, a derivative thereof, or a combination thereof.

14. The antibody or antigen binding fragment thereof of claim 1, wherein the antibody or antigen binding fragment thereof binds to the same epitope on the extracellular domain of the human ACE2 protein as does the receptor binding domain (RBD) of the SARS-CoV-2 spike protein, or the RBD of any other member of the betacoronavirus genus or sarbecovirus sub-genus that uses ACE2 to enter cells or any member of the alphacoronavirus genus.

15. The antibody or antigen binding fragment thereof of claim 14, wherein the member of the alphacoronavirus member is human coronavirus HCoV-NL-63.

16. An isolated nucleic acid encoding an immunoglobulin chain or variable region thereof of the antibody according to claim 1.

17. A vector comprising the isolated nucleic acid of claim 16.

18. A host cell comprising the isolated nucleic acid of claim 16.

19. The host cell of claim 18, wherein the host cell is a Expi 293 cell.

20. A method for making an antibody or antigen-binding fragment thereof according to claim 1 or an immunoglobulin chain thereof; comprising:
(a) introducing one or more nucleic acids encoding an immunoglobulin chain of antibody or antigen-binding fragment thereof into a host cell;
(b) culturing the host cell in a medium to express the immunoglobulin chain(s); and
(c) optionally, isolating the immunoglobulin chain or antibody or antigen-binding fragment thereof from the host cell and/or the medium.

21. The method of claim 20, wherein the host cell is a Expi 293 cell.

22. A pharmaceutical composition comprising an antibody or antigen-binding fragment thereof according to claim 1 and a pharmaceutically acceptable carrier.

23. A method for treating or preventing infection caused by coronavirus in a subject in need thereof, comprising administering to the subject a therapeutically effective amount of an antibody or antigen-binding fragment thereof according to claim 1.

24. The method of claim 23, wherein the coronavirus is a SARS-CoV, a SARS-CoV-2, a Pangolin CoV, a Bat CoV, any other member of the betacoronavirus genus or sarbecovirus sub-genus that uses ACE2 to enter cells or any.

25. The method of claim 24, wherein the member of the alphacoronavirus genus is human coronavirus HCoV-NL-63.

26. The method of claim 23, wherein the antibody or antigen-binding fragment thereof is injected into the subject subcutaneously, intravenously or intramuscularly.


SEQUENCE LISTING (96 sequences)

SEQ ID NO: 1 - protein, 967 aa, synthetic construct
  REGION 1..967: note = ACE2 (1-740aa)-Fc
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 2 - protein, 754 aa, synthetic construct
  REGION 1..754: note = ACE2 (1-740aa)-8His
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGSGGHHHHHHHH

SEQ ID NO: 3 - DNA, 696 bp, synthetic construct
  misc_feature 1..696: note = 05B04 - Light chain
  source 1..696: mol_type = other DNA
atgggatggtcatgtatcatcctttttctagtagcaactgcaaccggttccgatatccagatgacccagtctccttccgccatgagcgccagcgtgggcgatagagtgacaatcacctgcagagcctcccagggcatcagcaattacttcgcctggtttcagcagaaacctggcaaggtgcccaagcggctgatctacgccgcttctagcctgcagagcggcgtgccttctagattcagcggaagcggctctggcacagagttcaccctgaccattagcaacctgcaacctgaggacttcgctacatattactgcctgcaacacagctactacccctacaccttcggccagggcacaaagctcgagatcaagggaaccgtggccgcccctagcgtgttcatctttccaccttccgacgagcagctgaagtccggcacagcctctgtggtgtgcctgctgaacaacttctacccacgggaagccaaggtccagtggaaggtggacaacgccctgcagagcggcaacagccaggagagcgttaccgagcaggacagcaaggacagcacatactccctgagcagcaccctcaccctgtctaaagccgattacgagaagcacaaggtgtacgcttgtgaagtgacccaccagggcctgagcagccccgtgaccaagagcttcaatagaggagaatgctag

SEQ ID NO: 4 - protein, 231 aa, synthetic construct
  REGION 1..231: note = 05B04 - Light chain
MGWSCIILFLVATATGSDIQMTQSPSAMSASVGDRVTITCRASQGISNYFAWFQQKPGKVPKRLIYAASSLQSGVPSRFSGSGSGTEFTLTISNLQPEDFATYYCLQHSYYPYTFGQGTKLEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1413 bp, synthetic construct
  misc_feature 1..1413: note = 05B04 - Heavy chain
  source 1..1413: mol_type = other DNA
atgggatggtcatgtatcatcctttttctagtagcaactgcaaccggtgtacattctgaggtgcagctgctggagagcggcggaggcctggtgcaacctggaggaagcctgagactgagctgtgccgcttctggcttcaccttcagctcctacgccatgagctgggtgcggcaggcccctggcaagggcctggaatgggtctccggcatcagcggcagcggagatagaacatactatcctgacagcgtgaagggcagattcaccatcagcagagataacagcaaaaacaccctgtacctgcagatgaacagcctgcgggccgaggacaccgccgtgtactactgcgccaaggactgggccatggtgggcgccgatgccttcgacatctggggccccggaacaatggtgaccgtgagctccgcgtcgaccaagggccctagcgtctttccactggccccaagctctaagtccaccagcggcggcaccgcagctctgggctgcctggtgaaagactactttcctgagcctgtgacagtgtcttggaattccggtgctctgacaagcggcgtgcacacattccccgccgtgctgcagagctctggcctgtacagcctcagcagcgtggtgaccgttccttctagcagcctgggcacccagacatacatctgcaacgtgaaccacaaaccctctaataccaaggtggacaagaaggtggaacccaagtcttgcgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctccatgaggctctgcactcccactacacgcagaagagcctctccctgtctccgggtaaatga

SEQ ID NO: 6 - protein, 470 aa, synthetic construct
  REGION 1..470: note = 05B04 - Heavy chain
MGWSCIILFLVATATGVHSEVQLLESGGGLVQPGGSLRLSCAASGFTFSSYAMSWVRQAPGKGLEWVSGISGSGDRTYYPDSVKGRFTISRDNSKNTLYLQMNSLRAEDTAVYYCAKDWAMVGADAFDIWGPGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 7 - DNA, 696 bp, synthetic construct
  misc_feature 1..696: note = 05D06 Light chain
  source 1..696: mol_type = other DNA
atgggatggtcatgtatcatcctttttctagtagcaactgcaaccggttccgatatccagatgacccagagccctagtgctatgagcgcttctgtgggcgatagagtgacaatcacctgtagagcctcccagggcatcagcaattacctggcctggtttcagcagaaacctggcaaggtgcctaagcggctgatctacgccacaagctccctgcagtctggagtgccctctagattcagcggcagcggatctggcaccgaattcaccctgaccattagctccctgcaacctgaggacttcgccacctactactgcctccaacacagcaactacccttatacatttggccagggcacaaaactcgagatcaagggcaccgtggccgctcctagcgtgttcatcttcccccccagcgacgagcagctgaagagcggcaccgccagcgtggtgtgcctgctgaacaacttctaccctagagaagccaaggtccagtggaaggtggacaacgccctgcagagcggcaatagccaggagagcgtgaccgaacaggacagcaaagatagcacctacagcctgagctctacactgacactgagcaaggccgactacgagaagcacaaggtttacgcctgcgaggtgacccaccagggactttcttctccagtgaccaagtccttcaaccggggcgagtgttag

SEQ ID NO: 8 - protein, 231 aa, synthetic construct
  REGION 1..231: note = 05D06 Light chain
MGWSCIILFLVATATGSDIQMTQSPSAMSASVGDRVTITCRASQGISNYLAWFQQKPGKVPKRLIYATSSLQSGVPSRFSGSGSGTEFTLTISSLQPEDFATYYCLQHSNYPYTFGQGTKLEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 1413 bp, synthetic construct
  misc_feature 1..1413: note = 05D06 Heavy chain
  source 1..1413: mol_type = other DNA
atgggatggtcatgtatcatcctttttctagtagcaactgcaaccggtgtacattctgaggtgcagctcctggaaagcggcggcggcctggttcagcctggcggaagcctgcggctgagctgtgccgcttctggctttaccttcagcagctacgccatgagctgggtcagacaagctcccggaaaaggcctggaatgggtgctgggcatctctatcagcggcggatctacctatttcgccgatagcgtgaagggcagattcatcatctcccgggacaacagcaagtccaccctgtacctgcagatgaacagcctgagagccgaggatacagccgtgtactactgcgtgaaggactggtacattgtgggcgccgacgccttcgacatctggggccagggcaccatggtgacagtgtccgccgcgtcgaccaagggccctagcgtctttccactggccccaagctctaagtccaccagcggcggcaccgcagctctgggctgcctggtgaaagactactttcctgagcctgtgacagtgtcttggaattccggtgctctgacaagcggcgtgcacacattccccgccgtgctgcagagctctggcctgtacagcctcagcagcgtggtgaccgttccttctagcagcctgggcacccagacatacatctgcaacgtgaaccacaaaccctctaataccaaggtggacaagaaggtggaacccaagtcttgcgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctccatgaggctctgcactcccactacacgcagaagagcctctccctgtctccgggtaaatga

SEQ ID NO: 10 - protein, 470 aa, synthetic construct
  REGION 1..470: note = 05D06 Heavy chain
MGWSCIILFLVATATGVHSEVQLLESGGGLVQPGGSLRLSCAASGFTFSSYAMSWVRQAPGKGLEWVLGISISGGSTYFADSVKGRFIISRDNSKSTLYLQMNSLRAEDTAVYYCVKDWYIVGADAFDIWGQGTMVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 11 - DNA, 702 bp, synthetic construct
  misc_feature 1..702: note = 05E10 Light chain
  source 1..702: mol_type = other DNA
atgggatggtcatgtatcatcctttttctagtagcaactgcaaccggtgccaggtgtgacatccagatgacccagtctccatctgccatgtctgcatctgtaggagacagagtcaccatcacttgtcgggcgagtcagggcattagcaattatttagcctggtttcagcagaaaccagggaaagtccctaagcgcctgatctatgctacatccagtttgcaaagtggggtcccatcaaggttcagcggcagtggatctgggacagaattcactctcacaatcagcagcctgcagcctgaagattttgcaacttattactgtctacagcatagtaattacccgtacacttttggccaggggaccaagctcgagatcaagggaaccgtggccgcccctagcgtgttcatctttccaccttccgacgagcagctgaagtccggcacagcctctgtggtgtgcctgctgaacaacttctacccacgggaagccaaggtccagtggaaggtggacaacgccctgcagagcggcaacagccaggagagcgttaccgagcaggacagcaaggacagcacatactccctgagcagcaccctcaccctgtctaaagccgattacgagaagcacaaggtgtacgcttgtgaagtgacccaccagggcctgagcagccccgtgaccaagagcttcaatagaggagaatgctag

SEQ ID NO: 12 - protein, 233 aa, synthetic construct
  REGION 1..233: note = 05E10 Light chain
MGWSCIILFLVATATGARCDIQMTQSPSAMSASVGDRVTITCRASQGISNYLAWFQQKPGKVPKRLIYATSSLQSGVPSRFSGSGSGTEFTLTISSLQPEDFATYYCLQHSNYPYTFGQGTKLEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1413 bp, synthetic construct
  misc_feature 1..1413: note = 05E10 Heavy chain
  source 1..1413: mol_type = other DNA
atgggatggtcatgtatcatcctttttctagtagcaactgcaaccggtgtccagtgtgaggtgcagttgttggagtctgggggaggcttggtacagccgggggggtccctgagactctcctgtgcagcctctggattcacctttagcagctatgccatgagctgggtccgccaggctccagggaaggggctggagtgggtcttaggtatcaatataagtggtggtagtacatacttcgcagactccgtgaagggccggttcatcatctccagagacaattccaagagcacgctgtatctgcaaatgaacagcctgagagccgaggacacggccgtgtattactgtgtgaaagattggtatataatgggagccgatgcttttgatatctggggccaagggacaatggtcaccgtctctgcagcgtcgaccaagggccctagcgtctttccactggccccaagctctaagtccaccagcggcggcaccgcagctctgggctgcctggtgaaagactactttcctgagcctgtgacagtgtcttggaattccggtgctctgacaagcggcgtgcacacattccccgccgtgctgcagagctctggcctgtacagcctcagcagcgtggtgaccgttccttctagcagcctgggcacccagacatacatctgcaacgtgaaccacaaaccctctaataccaaggtggacaagaaggtggaacccaagtcttgcgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctccatgaggctctgcactcccactacacgcagaagagcctctccctgtctccgggtaaatga

SEQ ID NO: 14 - protein, 470 aa, synthetic construct
  REGION 1..470: note = 05E10 Heavy chain
MGWSCIILFLVATATGVQCEVQLLESGGGLVQPGGSLRLSCAASGFTFSSYAMSWVRQAPGKGLEWVLGINISGGSTYFADSVKGRFIISRDNSKSTLYLQMNSLRAEDTAVYYCVKDWYIMGADAFDIWGQGTMVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 15 - DNA, 702 bp, synthetic construct
  misc_feature 1..702: note = 05G01 Light chain
  source 1..702: mol_type = other DNA
atgggatggtcatgtatcatcctttttctagtagcaactgcaaccggtgccaggtgtgacatccagatgacccagtctccatctgccatgtctgcatctgttggagacagagtcaccatcacctgtcggacgagtcaggtcattagtaattattttgcctggtttcagcagaaaccagggagagtccctaagcgcctgatctatgctggatccactttgcaaagtggggtcccatcaaggttcagcggcggtggatctgggacagaattcactctcacaatcaccagcctgcagcctgaagattttgcaacttattactgtctacagcataataattacccgtacacttttggccaggggaccaagctcgagatcaagggaaccgtggccgcccctagcgtgttcatctttccaccttccgacgagcagctgaagtccggcacagcctctgtggtgtgcctgctgaacaacttctacccacgggaagccaaggtccagtggaaggtggacaacgccctgcagagcggcaacagccaggagagcgttaccgagcaggacagcaaggacagcacatactccctgagcagcaccctcaccctgtctaaagccgattacgagaagcacaaggtgtacgcttgtgaagtgacccaccagggcctgagcagccccgtgaccaagagcttcaatagaggagaatgctag

SEQ ID NO: 16 - protein, 233 aa, synthetic construct
  REGION 1..233: note = 05G01 Light chain
MGWSCIILFLVATATGARCDIQMTQSPSAMSASVGDRVTITCRTSQVISNYFAWFQQKPGRVPKRLIYAGSTLQSGVPSRFSGGGSGTEFTLTITSLQPEDFATYYCLQHNNYPYTFGQGTKLEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 1413 bp, synthetic construct
  misc_feature 1..1413: note = 05G01 Heavy chain
  source 1..1413: mol_type = other DNA
atgggatggtcatgtatcatcctttttctagtagcaactgcaaccggtgtccagtgtgaggtgcaactgttggagtctgggggaggcttggtacagccgggggggtccctgagactctcctgtgcagcctctggattcacctttagcaactatgccatgagctgggtccgccaggctccagggaaggggctggaatgggtctcaggtattagtattaatggtgataggacatactacgcagactccgtgaagggccggttcaccgtctccagagactattccaagaacacgctgtatctgcaaatgaacagcctgagagccgaggacacggccgtatattattgtgcgaaagactgggcgatagtgggagccgatgcttttgatgtctggggccaagggacaatggtcaccgtctcttcagcgtcgaccaagggccctagcgtctttccactggccccaagctctaagtccaccagcggcggcaccgcagctctgggctgcctggtgaaagactactttcctgagcctgtgacagtgtcttggaattccggtgctctgacaagcggcgtgcacacattccccgccgtgctgcagagctctggcctgtacagcctcagcagcgtggtgaccgttccttctagcagcctgggcacccagacatacatctgcaacgtgaaccacaaaccctctaataccaaggtggacaagaaggtggaacccaagtcttgcgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctccatgaggctctgcactcccactacacgcagaagagcctctccctgtctccgggtaaatga

SEQ ID NO: 18 - protein, 470 aa, synthetic construct
  REGION 1..470: note = 05G01 Heavy chain
MGWSCIILFLVATATGVQCEVQLLESGGGLVQPGGSLRLSCAASGFTFSNYAMSWVRQAPGKGLEWVSGISINGDRTYYADSVKGRFTVSRDYSKNTLYLQMNSLRAEDTAVYYCAKDWAIVGADAFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 19 - DNA, 702 bp, synthetic construct
  misc_feature 1..702: note = 05H02 Light chain
  source 1..702: mol_type = other DNA
atgggatggtcatgtatcatcctttttctagtagcaactgcaaccggtgccaaatgtgacatccagatgacccagtctccttccaccctgtctgcatctgtaggagacagagtcaccatcacttgccgggccagtcagagtattagtagttggttggcctggtatcagcagaaaccagggaaagcccctaagctcctaatctataaggcgtctagtttagaaagtggggtcccatcaaggttcagcggcagtggatctgggacagaattcactctcaccatcagcagcctgcagcctgatgattttgcaacttatttctgccaacagtataatacttattctcggacgttcggcccagggaccaagctcgagatcaagggaaccgtggccgcccctagcgtgttcatctttccaccttccgacgagcagctgaagtccggcacagcctctgtggtgtgcctgctgaacaacttctacccacgggaagccaaggtccagtggaaggtggacaacgccctgcagagcggcaacagccaggagagcgttaccgagcaggacagcaaggacagcacatactccctgagcagcaccctcaccctgtctaaagccgattacgagaagcacaaggtgtacgcttgtgaagtgacccaccagggcctgagcagccccgtgaccaagagcttcaatagaggagaatgctag

SEQ ID NO: 20 - protein, 233 aa, synthetic construct
  REGION 1..233: note = 05H02 Light chain
MGWSCIILFLVATATGAKCDIQMTQSPSTLSASVGDRVTITCRASQSISSWLAWYQQKPGKAPKLLIYKASSLESGVPSRFSGSGSGTEFTLTISSLQPDDFATYFCQQYNTYSRTFGPGTKLEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 1404 bp, synthetic construct
  misc_feature 1..1404: note = 05H02 Heavy chain
  source 1..1404: mol_type = other DNA
atgggatggtcatgtatcatcctttttctagtagcaactgcaaccggtgtccagtgtgaggtgctgctgttggagtctgggggaggcttggtgcagcctggggggtccctgagcctctcctgtacagcctctggattcacctttaacatctatgccatgaactgggtccgccaggctccagggaaggggctggagtgggtctcaacttttagtggtagtcgttataacacttactacgcagactccgtgaagggccggttcaccatctccagagacaattccaagaactcgctgtatctgcaaatgaacagcctgagagccgaggacacggccgtatatttctgtgcgaaagaagcagtggctgggcagtttgactactggggccagggaaccctggtcaccgtctcctcagcgtcgaccaagggccctagcgtctttccactggccccaagctctaagtccaccagcggcggcaccgcagctctgggctgcctggtgaaagactactttcctgagcctgtgacagtgtcttggaattccggtgctctgacaagcggcgtgcacacattccccgccgtgctgcagagctctggcctgtacagcctcagcagcgtggtgaccgttccttctagcagcctgggcacccagacatacatctgcaacgtgaaccacaaaccctctaataccaaggtggacaagaaggtggaacccaagtcttgcgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctccatgaggctctgcactcccactacacgcagaagagcctctccctgtctccgggtaaatga

SEQ ID NO: 22 - protein, 467 aa, synthetic construct
  REGION 1..467: note = 05H02 Heavy chain
MGWSCIILFLVATATGVQCEVLLLESGGGLVQPGGSLSLSCTASGFTFNIYAMNWVRQAPGKGLEWVSTFSGSRYNTYYADSVKGRFTISRDNSKNSLYLQMNSLRAEDTAVYFCAKEAVAGQFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 23 - DNA, 708 bp, synthetic construct
  misc_feature 1..708: note = 2G7A1 Light chain
  source 1..708: mol_type = other DNA
atgggatggtcatgtatcatcctttttctagtagcaactgcaaccggttcttgggccaattttatgttgactcagccccactctgtgtcggagtctccggggaagacggtaaccatctcctgcacccgcagcagtggcagcattaacaacaactatgtgcagtggttccaacagcgccccggcagttcccccaccactgtgatctatgacgataaccacagaccctctggggtccctgatcggttctctggctccatcgacaggtcctccaactctgcctccctcaccatctctggactgaagactgaggacgaggctgactactactgtcagtcttatgatagcagcagtcgggtgttcggcggagggaccaaactgaccgtcctaggtcagcccaaggctgccccctcggtcactctgttcccgccctcgagtgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 24 - protein, 235 aa, synthetic construct
  REGION 1..235: note = 2G7A1 Light chain
MGWSCIILFLVATATGSWANFMLTQPHSVSESPGKTVTISCTRSSGSINNNYVQWFQQRPGSSPTTVIYDDNHRPSGVPDRFSGSIDRSSNSASLTISGLKTEDEADYYCQSYDSSSRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - DNA, 1401 bp, synthetic construct
  misc_feature 1..1401: note = 2G7A1 Heavy chain
  source 1..1401: mol_type = other DNA
atgggatggtcatgtatcatcctttttctagtagcaactgcaaccggtgtcctgtcccaggtgcaactacaggagtcgggcccaggactggtgaagccttcggagaccctgtccctcacctgcactgtctctgatggctccatcagtggttactactggaattggatccggcagtccccagggaagggactgaaatggattggatatatccattacagtgggaccaccacctacgactcctccctcaagagtcgagtcaccttatcagtggacatgtccaagaaccagttctccctgaagatgacctctgtgaccgttgcagactcggccgtgtattactgtgcgagaggcccagttttctggtacttcgatctctggggccatggcaccctggtcactgtctcctcagcgtcgaccaagggccctagcgtctttccactggccccaagctctaagtccaccagcggcggcaccgcagctctgggctgcctggtgaaagactactttcctgagcctgtgacagtgtcttggaattccggtgctctgacaagcggcgtgcacacattccccgccgtgctgcagagctctggcctgtacagcctcagcagcgtggtgaccgttccttctagcagcctgggcacccagacatacatctgcaacgtgaaccacaaaccctctaataccaaggtggacaagaaggtggaacccaagtcttgcgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctccatgaggctctgcactcccactacacgcagaagagcctctccctgtctccgggtaaatga

SEQ ID NO: 26 - protein, 466 aa, synthetic construct
  REGION 1..466: note = 2G7A1 Heavy chain
MGWSCIILFLVATATGVLSQVQLQESGPGLVKPSETLSLTCTVSDGSISGYYWNWIRQSPGKGLKWIGYIHYSGTTTYDSSLKSRVTLSVDMSKNQFSLKMTSVTVADSAVYYCARGPVFWYFDLWGHGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 27 - DNA, 708 bp, synthetic construct
  misc_feature 1..708: note = 2F6A6 Light chain
  source 1..708: mol_type = other DNA
atgggatggtcatgtatcatcctttttctagtagcaactgcaaccggttcttgggccaattttatattgactcagccccactctgtgtcggagtctccggggaagacggtaaccatctcctgcacccgcagcagtggcagcattagcaacagctatgtgcagtggttccaacagcgccccggcagttcccccaccactgtgatctatgacgataaccacagaccctctggggtccctgatcggttctctggctccatcgacaggtcctccaactctgcctccctcaccatctctggactgaagactgaggacgaggctgactactactgtcagtcttatgatagcagcagtcgggtgttcggcggagggaccaagctgaccgtcctaggtcagcccaaggctgccccctcggtcactctgttcccgccctcgagtgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 28 - protein, 235 aa, synthetic construct
  REGION 1..235: note = 2F6A6 Light chain
MGWSCIILFLVATATGSWANFILTQPHSVSESPGKTVTISCTRSSGSISNSYVQWFQQRPGSSPTTVIYDDNHRPSGVPDRFSGSIDRSSNSASLTISGLKTEDEADYYCQSYDSSSRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 29 - DNA, 1401 bp, synthetic construct
  misc_feature 1..1401: note = 2F6A6 Heavy chain
  source 1..1401: mol_type = other DNA
atgggatggtcatgtatcatcctttttctagtagcaactgcaaccggtgtcctgtcccaggtgcaactgcaggagtcgggcccaggactggtgaagccttcggagaccctgtccctcacctgcactgtctctgatggctccatcagtggttattactggaactggatccggcagcccccagggaagggactggaatggattggatatatctattacagtgggaccaccacctacaacccctccctcaagagtcgagtcaccttatcagtggacacgtccaagaatcagttctccctgaagttgacctctgtgaccgctgcagactcggccgtgtattactgtgcgagaggcccagttttctggtacttcgatctctggggccgtggcaccctggtcactgtctcctcagcgtcgaccaagggccctagcgtctttccactggccccaagctctaagtccaccagcggcggcaccgcagctctgggctgcctggtgaaagactactttcctgagcctgtgacagtgtcttggaattccggtgctctgacaagcggcgtgcacacattccccgccgtgctgcagagctctggcctgtacagcctcagcagcgtggtgaccgttccttctagcagcctgggcacccagacatacatctgcaacgtgaaccacaaaccctctaataccaaggtggacaagaaggtggaacccaagtcttgcgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctccatgaggctctgcactcccactacacgcagaagagcctctccctgtctccgggtaaatga

SEQ ID NO: 30 - protein, 466 aa, synthetic construct
  REGION 1..466: note = 2F6A6 Heavy chain
MGWSCIILFLVATATGVLSQVQLQESGPGLVKPSETLSLTCTVSDGSISGYYWNWIRQPPGKGLEWIGYIYYSGTTTYNPSLKSRVTLSVDTSKNQFSLKLTSVTAADSAVYYCARGPVFWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 31 - DNA, 702 bp, synthetic construct
  misc_feature 1..702: note = 4A12A4 Light chain
  source 1..702: mol_type = other DNA
atgggatggtcatgtatcatcctttttctagtagcaactgcaaccggttctgtggtttcttctgagctgactcaggaccctgctgtgtctgtggccttgggacagacagtcaggatcacatgccaaggagacagcctcagaaactattatgccagctggtaccagcagaagccaggacaggcccctgtacttgtcatctttggtaaaaacaaccggccctcagggatcccagaccgattctctggctccagctcagggaacacggcttccttgaccatcactggggctcaggcggaagatgaggctgactattactgtaactcccgggacagcggtggtaaccatgtggtattcggcggagggaccaaactgaccgtcctaggtcagcccaaggctgccccctcggtcactctgttcccgccctcgagtgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 32 - protein, 233 aa, synthetic construct
  REGION 1..233: note = 4A12A4 Light chain
MGWSCIILFLVATATGSVVSSELTQDPAVSVALGQTVRITCQGDSLRNYYASWYQQKPGQAPVLVIFGKNNRPSGIPDRFSGSSSGNTASLTITGAQAEDEADYYCNSRDSGGNHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 33 - DNA, 1404 bp, synthetic construct
  misc_feature 1..1404: note = 4A12A4 Heavy chain
  source 1..1404: mol_type = other DNA
atgggatggtcatgtatcatcctttttctagtagcaactgcaaccggtgtccaatgtgaggaccagctggtggagtctgggggaggcctggtcaagcctggggggtccctgagactctcctgtgcagcctctggattcaccttcagtagctatagcatgaactgggtccgccaggctccagggaagggactggagtgggtctcatccattaccaatggtattaataacatacactacgcagactcagtgaagggccgattcaccatctccagagacatcgccaaaaattcactgtatctgcaaatgaacggcctgagagccgaggacacggctgtgtattactgtgcgagatactattactactacggtatggacgtctggggccaagggaccacggtctccgtctcctcagcgtcgaccaagggccctagcgtctttccactggccccaagctctaagtccaccagcggcggcaccgcagctctgggctgcctggtgaaagactactttcctgagcctgtgacagtgtcttggaattccggtgctctgacaagcggcgtgcacacattccccgccgtgctgcagagctctggcctgtacagcctcagcagcgtggtgaccgttccttctagcagcctgggcacccagacatacatctgcaacgtgaaccacaaaccctctaataccaaggtggacaagaaggtggaacccaagtcttgcgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctccatgaggctctgcactcccactacacgcagaagagcctctccctgtctccgggtaaatga

SEQ ID NO: 34 - protein, 467 aa, synthetic construct
  REGION 1..467: note = 4A12A4 Heavy chain
MGWSCIILFLVATATGVQCEDQLVESGGGLVKPGGSLRLSCAASGFTFSSYSMNWVRQAPGKGLEWVSSITNGINNIHYADSVKGRFTISRDIAKNSLYLQMNGLRAEDTAVYYCARYYYYYGMDVWGQGTTVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 35 - DNA, 696 bp, synthetic construct
  misc_feature 1..696: note = 2C12H3 Light chain
  source 1..696: mol_type = other DNA
atgggatggtcatgtatcatcctttttctagtagcaactgcaaccggtgaaactgtgttgacgcagtctccagacaccctgtctttgtctccaggggaaagagccaccctctcctgcagggccagtcagagtgttagcagcagctacttagcctggtaccagcagaaacctggccaggctcccaggctcctcatctatggtgcatccagcagggcctctggcatcccagacaggttcagtggcagtgggtctgggacagacttcactctcaccatcaacagactggagcctgaagatttttcagtgtattactgtcagcagtatggtagttcacctcggacgttcggccaagggaccaagctcgagatcaagggaaccgtggccgcccctagcgtgttcatctttccaccttccgacgagcagctgaagtccggcacagcctctgtggtgtgcctgctgaacaacttctacccacgggaagccaaggtccagtggaaggtggacaacgccctgcagagcggcaacagccaggagagcgttaccgagcaggacagcaaggacagcacatactccctgagcagcaccctcaccctgtctaaagccgattacgagaagcacaaggtgtacgcttgtgaagtgacccaccagggcctgagcagccccgtgaccaagagcttcaatagaggagaatgctag

SEQ ID NO: 36 - protein, 231 aa, synthetic construct
  REGION 1..231: note = 2C12H3 Light chain
MGWSCIILFLVATATGETVLTQSPDTLSLSPGERATLSCRASQSVSSSYLAWYQQKPGQAPRLLIYGASSRASGIPDRFSGSGSGTDFTLTINRLEPEDFSVYYCQQYGSSPRTFGQGTKLEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 1401 bp, synthetic construct
  misc_feature 1..1401: note = 2C12H3 Heavy chain
  source 1..1401: mol_type = other DNA
atgggatggtcatgtatcatcctttttctagtagcaactgcaaccggtgtcctgtcccaggtgcagttgcaggagtcgggcccaggactggtgaagccttcggagaccctgtccctcacctgcactgtctctggtggctccatcactagttactacttcagctggatccggcagcccgccgggaagggactggagtggattgggcgcatcttttccagtgggatcaccaactacaacccctccctcaagagtcgagtcaccatgtcagtggacacgtccaagaaccggttctccctgaaactgaactctgtgaccgccgcggacacggccgtatattactgtgcgagacacgatggtttcgggtggttcgacccctggggccagggaaccctggtcaccgtctcctcagcgtcgaccaagggccctagcgtctttccactggccccaagctctaagtccaccagcggcggcaccgcagctctgggctgcctggtgaaagactactttcctgagcctgtgacagtgtcttggaattccggtgctctgacaagcggcgtgcacacattccccgccgtgctgcagagctctggcctgtacagcctcagcagcgtggtgaccgttccttctagcagcctgggcacccagacatacatctgcaacgtgaaccacaaaccctctaataccaaggtggacaagaaggtggaacccaagtcttgcgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctccatgaggctctgcactcccactacacgcagaagagcctctccctgtctccgggtaaatga

SEQ ID NO: 38 - protein, 466 aa, synthetic construct
  REGION 1..466: note = 2C12H3 Heavy chain
MGWSCIILFLVATATGVLSQVQLQESGPGLVKPSETLSLTCTVSGGSITSYYFSWIRQPAGKGLEWIGRIFSSGITNYNPSLKSRVTMSVDTSKNRFSLKLNSVTAADTAVYYCARHDGFGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 39 - DNA, 708 bp, synthetic construct
  misc_feature 1..708: note = 1C9H1 Light chain
  source 1..708: mol_type = other DNA
atgggatggtcatgtatcatcctttttctagtagcaactgcaaccggttcctgggcccagtctgccctgactcagcctgcctccgtgtctgggtctcctggacagtcgatcaccatctcctgcactggaaccagcagtgatgttggaagttataaccttgtctcctggtaccaacagcacccagacaaagcccccaaactcatgatttatgagggcagtaagcggccctcaggggtttctaatcgcttctctggctccaagtctggcaacacggcctccctgacaatctctgggctccaggctgaggacgaggctgattattactgctgctcatatgcaggtagtaacattgttgtattcggcggagggaccaagctgaccgtcctaggtcagcccaaggctgccccctcggtcactctgttcccgccctcgagtgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 40 - protein, 235 aa, synthetic construct
  REGION 1..235: note = 1C9H1 Light chain
MGWSCIILFLVATATGSWAQSALTQPASVSGSPGQSITISCTGTSSDVGSYNLVSWYQQHPDKAPKLMIYEGSKRPSGVSNRFSGSKSGNTASLTISGLQAEDEADYYCCSYAGSNIVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - DNA, 1407 bp, synthetic construct
  misc_feature 1..1407: note = 1C9H1 Heavy chain
  source 1..1407: mol_type = other DNA
atgggatggtcatgtatcatcctttttctagtagcaactgcaaccggtgtcttgtcccagatcaccttgaaggagtctggtcctacgctggtgaaacccacacagaccctcacgctgacctgcaccttctctgggttctcactcagcactcctggagtggctgtgggctggatccgtcagcccccaggagaggccctggagtggcttgcattcattttttggaatgatgatgagcgctacagcccatctctgaggagcaggctcaccatcaccaaagacacctccaaaaaccaggtggtccttacaatgaccaacatggaccctattgacacagccactttttactgtgcacacctagccctctactggtacttcgatttttggggccgtggcaccctggtcactgtctcctcagcgtcgaccaagggccctagcgtctttccactggccccaagctctaagtccaccagcggcggcaccgcagctctgggctgcctggtgaaagactactttcctgagcctgtgacagtgtcttggaattccggtgctctgacaagcggcgtgcacacattccccgccgtgctgcagagctctggcctgtacagcctcagcagcgtggtgaccgttccttctagcagcctgggcacccagacatacatctgcaacgtgaaccacaaaccctctaataccaaggtggacaagaaggtggaacccaagtcttgcgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctccatgaggctctgcactcccactacacgcagaagagcctctccctgtctccgggtaaatga

SEQ ID NO: 42 - protein, 468 aa, synthetic construct
  REGION 1..468: note = 1C9H1 Heavy chain
MGWSCIILFLVATATGVLSQITLKESGPTLVKPTQTLTLTCTFSGFSLSTPGVAVGWIRQPPGEALEWLAFIFWNDDERYSPSLRSRLTITKDTSKNQVVLTMTNMDPIDTATFYCAHLALYWYFDFWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 43 - protein, 8 aa, synthetic construct
  REGION 1..8: note = 05B04 Heavy chain CDR-1
GFTFSSYA

SEQ ID NO: 44 - protein, 8 aa, synthetic construct
  REGION 1..8: note = 05B04 Heavy chain CDR-2
ISGSGDRT

SEQ ID NO: 45 - protein, 14 aa, synthetic construct
  REGION 1..14: note = 05B04 Heavy chain CDR-3
AKDWAMVGADAFDI

SEQ ID NO: 46 - protein, 6 aa, synthetic construct
  REGION 1..6: note = 05B04 Light chain CDR-1
QGISNY

SEQ ID NO: 47 - protein, 9 aa, synthetic construct
  REGION 1..9: note = 05B04 Light chain CDR-3
LQHSYYPYT

SEQ ID NO: 48 - protein, 8 aa, synthetic construct
  REGION 1..8: note = 05D06 Heavy chain CDR-1
GFTFSSYA

SEQ ID NO: 49 - protein, 8 aa, synthetic construct
  REGION 1..8: note = 05D06 Heavy chain CDR-2
ISISGGST

SEQ ID NO: 50 - protein, 14 aa, synthetic construct
  REGION 1..14: note = 05D06 Heavy chain CDR-2
VKDWYIVGADAFDI

SEQ ID NO: 51 - protein, 6 aa, synthetic construct
  REGION 1..6: note = 05D06 Light chain CDR-1
QGISNY

SEQ ID NO: 52 - protein, 9 aa, synthetic construct
  REGION 1..9: note = 05D06 Light chain CDR-3
LQHSNYPYT

SEQ ID NO: 53 - protein, 8 aa, synthetic construct
  REGION 1..8: note = 05E10 Heavy chain CDR-1
GFTFSSYA

SEQ ID NO: 54 - protein, 8 aa, synthetic construct
  REGION 1..8: note = 05E10 Heavy chain CDR-2
INISGGST

SEQ ID NO: 55 - protein, 14 aa, synthetic construct
  REGION 1..14: note = 05E10 Heavy chain CDR-3
VKDWYIMGADAFDI

SEQ ID NO: 56 - protein, 6 aa, synthetic construct
  REGION 1..6: note = 05E10 Light chain CDR-1
QGISNY

SEQ ID NO: 57 - protein, 9 aa, synthetic construct
  REGION 1..9: note = 05E10 Light chain CDR-3
LQHSNYPYT

SEQ ID NO: 58 - protein, 8 aa, synthetic construct
  REGION 1..8: note = 05G01 Heavy chain CDR-1
GFTFSNYA

SEQ ID NO: 59 - protein, 8 aa, synthetic construct
  REGION 1..8: note = 05G01 Heavy chain CDR-2
ISINGDRT

SEQ ID NO: 60 - protein, 14 aa, synthetic construct
  REGION 1..14: note = 05G01 Heavy chain CDR-3
AKDWAIVGADAFDV

SEQ ID NO: 61 - protein, 6 aa, synthetic construct
  REGION 1..6: note = 05G01 Light chain CDR-1
QVISNY

SEQ ID NO: 62 - protein, 9 aa, synthetic construct
  REGION 1..9: note = 05G01 Light chain CDR-3
LQHNNYPYT

SEQ ID NO: 63 - protein, 8 aa, synthetic construct
  REGION 1..8: note = 05H02 Heavy chain CDR-1
GFTFNIYA

SEQ ID NO: 64 - protein, 8 aa, synthetic construct
  REGION 1..8: note = 05H02 Heavy chain CDR-2
FSGSRYNT

SEQ ID NO: 65 - protein, 11 aa, synthetic construct
  REGION 1..11: note = 05H02 Heavy chain CDR-3
AKEAVAGQFDY

SEQ ID NO: 66 - protein, 6 aa, synthetic construct
  REGION 1..6: note = 05H02 Light chain CDR-1
QSISSW

SEQ ID NO: 67 - protein, 9 aa, synthetic construct
  REGION 1..9: note = 05H02 Light chain CDR-3
QQYNTYSRT

SEQ ID NO: 68 - protein, 8 aa, synthetic construct
  REGION 1..8: note = 2G7A1 Heavy chain CDR-1
DGSISGYY

SEQ ID NO: 69 - protein, 7 aa, synthetic construct
  REGION 1..7: note = 2G7A1 Heavy chain CDR-2
IHYSGTT

SEQ ID NO: 70 - protein, 11 aa, synthetic construct
  REGION 1..11: note = 2G7A1 Heavy chain CDR-3
ARGPVFWYFDL

SEQ ID NO: 71 - protein, 8 aa, synthetic construct
  REGION 1..8: note = 2G7A1 Light chain CDR-1
SGSINNNY

SEQ ID NO: 72 - protein, 9 aa, synthetic construct
  REGION 1..9: note = 2G7A1 Light chain CDR-3
QSYDSSSRV

SEQ ID NO: 73 - protein, 8 aa, synthetic construct
  REGION 1..8: note = 2F6A6 Heavy chain CDR-1
DGSISGYY

SEQ ID NO: 74 - protein, 7 aa, synthetic construct
  REGION 1..7: note = 2F6A6 Heavy chain CDR-2
IYYSGTT

SEQ ID NO: 75 - protein, 11 aa, synthetic construct
  REGION 1..11: note = 2F6A6 Heavy chain CDR-3
ARGPVFWYFDL

SEQ ID NO: 76 - protein, 8 aa, synthetic construct
  REGION 1..8: note = 2F6A6 Light chain CDR-1
SGSISNSY

SEQ ID NO: 77 - protein, 9 aa, synthetic construct
  REGION 1..9: note = 2F6A6 Light chain CDR-3
QSYDSSSRV

SEQ ID NO: 78 - protein, 8 aa, synthetic construct
  REGION 1..8: note = 4A12A4 Heavy chain CDR-1
GFTFSSYS

SEQ ID NO: 79 - protein, 8 aa, synthetic construct
  REGION 1..8: note = 4A12A4 Heavy chain CDR-2
ITNGINNI

SEQ ID NO: 80 - protein, 11 aa, synthetic construct
  REGION 1..11: note = 4A12A4 Heavy chain CDR-3
ARYYYYYGMDV

SEQ ID NO: 81 - protein, 6 aa, synthetic construct
  REGION 1..6: note = 4A12A4 Light chain CDR-1
SLRNYY

SEQ ID NO: 82 - protein, 11 aa, synthetic construct
  REGION 1..11: note = 4A12A4 Light chain CDR-3
NSRDSGGNHVV

SEQ ID NO: 83 - protein, 8 aa, synthetic construct
  REGION 1..8: note = 2C12H3 Heavy chain CDR-1
GGSITSYY

SEQ ID NO: 84 - protein, 8 aa, synthetic construct
  REGION 1..8: note = 2C12H3 Heavy chain CDR-1
GGSITSYY

SEQ ID NO: 85 - protein, 11 aa, synthetic construct
  REGION 1..11: note = 2C12H3 Heavy chain CDR-3
ARHDGFGWFDP

SEQ ID NO: 86 - protein, 7 aa, synthetic construct
  REGION 1..7: note = 2C12H3 Light chain CDR-1
QSVSSSY

SEQ ID NO: 87 - protein, 9 aa, synthetic construct
  REGION 1..9: note = 2C12H3 Light chain CDR-3
QQYGSSPRT

SEQ ID NO: 88 - protein, 10 aa, synthetic construct
  REGION 1..10: note = 1C9H1 Heavy chain CDR-1
GFSLSTPGVA

SEQ ID NO: 89 - protein, 7 aa, synthetic construct
  REGION 1..7: note = 1C9H1 Heavy chain CDR-2
IFWNDDE

SEQ ID NO: 90 - protein, 11 aa, synthetic construct
  REGION 1..11: note = 1C9H1 Heavy chain CDR-3
AHLALYWYFDF

SEQ ID NO: 91 - protein, 9 aa, synthetic construct
  REGION 1..9: note = 1C9H1 Light chain CDR-1
SSDVGSYNL

SEQ ID NO: 92 - protein, 10 aa, synthetic construct
  REGION 1..10: note = 1C9H1 Light chain CDR-3
CSYAGSNIVV

SEQ ID NO: 93 - DNA, 89015 bp, synthetic construct
  misc_feature 1..89015: note = ACE2
  source 1..89015: mol_type = other DNA
ttagaactttttaaaagaggcaaaggcagaggagaacaaaggaaggaggaagtaacttgtggaatgttgagaaaggtaaaaaccccttcaaataaggaagaggaacaggctatgacctaatgcttgtttggagcagtattaaggatgccagggcaaatatttaggttaaattgtgggagctaagaacataaagtacattgatttttttttattatggctagcagatatttaagaatgttagcacaggtttttgaatgaattttgcttctaagagaagctattatttattcctaattagacagggaggaaagtctttgaagaggaacctctactttactttttacatttaggaaaaaagggactgctatgcaactaaaaatgtaaacaggaaagagctttagcttttcctttgggctgaaagcccaactagttatttattttctataaagcacttataaattcactttgtgtgataaacatgtggtcaaaatcaattgtactgaatgcaaatagttcttgaccatgcaaagttttgaatttctctatgcattgtgttcctgatacccttccttgttctgtcagcagtattaatcaataagaaattgaacaaaacatcctttcttttctgatgtttaccaaagttatccacccaacttggggaactggaagaggtttctcctatagctatttggcaacctcagcatttgtgactcagttggaaggatataaaaagttgaacagtaagggaaatatggatttcaaagtctatgtactttgctctagcagaataaagacccaccctcggatttcagcctatgaaaagggaggtttctagttgaactccatggttatctagtttccatttaggttttagcagatagcagatcagaaacagggaggggacttgtggagagcagggctactgaaagttgatactgtgactgtgaactcagaatgtgcagaaacagcagaattaatagcaataaagctgttgagtcatttaatgtaaagccttgaagacattgtcacattatagcttagccagtaaatattatttaagatgatgacactgagtcatctgaaatataaccctaaactaatgtccaggcagcatcgcgatcgggatggaatatattgaagaaaaatttctaactaaaacggtttaaaaatgtataactggtgtaatcaaagcagtacattttaattttgcttatggggacctgcttattctcagaatatgacagattgatgaggacttattagacatgcatgaagttagcagataactgaacccatgactctgtggagagaaaaggtctcttaagcaatctgggatcaaaatgtagccccattgcctatcagttgtggaagtttgaattaatcatgggaacagttggtttccttaattataaaatggatctcactatttctacctgtaaagttgttgtgaagagtttatatacaggagcaggtacaaagcatatgcaaccaacaaatacaacatctgtaaatattgagagggagtcatttacctttatgactttccccttcttcctcaagatagcgtgacagactcaagttttctcttctctgtatgtaggaagttttataccaggacctgaccatgactgggtgatgaaagcacgctgagaggttctctatggctgggctcatgtagactactggctgactcccctttcctttcatatatgactctactcattccattggtgagctcctccatcctgctgtttcagcacaaactagcagacttctcctacaatgcaggatcctatagtccaaacagacctttgagtcaaactcccagccagtctctgtctgctgcagtgtctctgaaagatgaccttgcagatggtttgattaaacattccagtgtggaagttctctaattattacacagctcctttcattttctgtggcttttattactaggcttacaacattactctctgtgttcttatgtattaattcaagtaatatgacatccctatgaaatggatataaaactatcattggccttgaaataaattaggtgcagggaggatattacctgcctaaggtcacacagctaacaagtggcagagtaaatccactatgctaagtcttcttctaaatggcagtaccatgatggaagaaagctgtccaccctgtcttctcttccatatctaaaccattctccaaattatattattttagatccatctgctctcctatttattttcctctggatgctctcttgtgcgtctctaaaattgtggttcacagaaatgaatgttatagtctagattattttgtatctaatgcttatattagcaaagtctaatatagtataagatgtaattataggcccagtaatttttactgggcctcctagttggatcttgaagctcatactaagcttttattcaaccagcttgactgggctgctgcagtagctcattaactagcctccctgcttccatccttacccctttagtgtccattctctaagcagcagccaaagtgaccctgctaaagcttctactagatcacttctctgcttgaagccttccagtgtcttcccatgttgctgagagtaaagctgaagtctcacagttaattgtctttaagacctccctggtgtgaccctagcttcttccttgacttcacctcctgccactttcccctgcacactgagctccagtcacaaagacctctgatgttcttgaaatctgcctacatcctttacacttgctgctccctctccctaggaaaacttcttcacccagagatccgcacaactttcatcacttctttccggtctttgctcaaacgtcaccttctcagagaggccctccttatagaaaatccctctccccaccccataattctctatcctccatttcttgcttattataagtcatgacatttatcactatttttgtcttccttctattgtcacctcatataatacaatatttattagcttacattcaaaatctagaatagtgtttggcatatagtaggtattcaataaacaatgattgaagcaagattcttgttgttttggacatgaattgttgtttaccagaggactgcctaatcctgtactgtgtaattgtttcaggggacatttgggtctagtcatagcattttacattctgttaaatcccttattgaaaaggaagatacactacgtctttggatatatttttttactattgtattctatcagagaggagatagggcaggcagcatctgacttattcctagtgaattcctaatggaccagagatatatttttccaagtgcaaatcacattctaaaaattttactttaggccgggtgcggtggctcatgcctgtaatcccagcactttgggaggccgaggcgggcagatcacgaggtcagaagatcgagaccatcctggctaacacagtgaaaccccgtctctattaaaaatacaaaaatattagctgcgcatggtggcgggcgcctgtagtcccagctactcgggaggccgaggcaggagaatggcgtgaacctgggaggcggagcttgcagtgagcggagatcgcgccgctgcactccagcctgggtgacaaagcgagactccgtctcaaaaaaaaaaaaatttactttatagctttactaggtgttaaggtcaaacttccctttaccattatgaaatccaccccctcctccaatctttttgaaacatagaacatctttcctgcatctttggtcttctaattttttctgattctccaatttttctcaaagacaatacattcctttagaccaattgtttttctgggtctcatctcctgagtcttcagtgaattctttccattctcggaatgcgctcagagagaacgcttctggggaactatcagaacattgatatcttctcaaaaaagtgtgaacaaaaattccaataattcagtcagtgcttgcagttggccattctgggctagtttttctacagaatcactgtagactattctcccttttcccttgcttctcaaagtttcttggcttctagcattttcctgcctgcctcactgctgtcccaggctccttgttcatttattcttagctttcagattttatatcttgctttctccatttcagtttgatcaagttaatcgtattttctgcctcagcgaattgaatttggactactcccattttttccactaaacccacgtgtttcatttaggaacttacctgtgtagaaatttatttccgcttttattacagtaacatttcaaccttttcttcagatactcgtgtgctgtcctctttcttgcctgttttttctcctattgtgtgcaaggagtgagtggatctaggtttcctggaatgtgggaggagcttttctgaaatttctgcactgattttttttttttaccctcatctcactttcatttaaaattctttattattattacttatatttgtgagctgctttatttttttaaaaggagagtatattttgattcacatcaagggaagtaagagtccacctctttttgatgcagtgtaactaaatctgtcatcttcactgaggcacaaggccctctgtcacattttatctatgcctccactattgcttttagactgttgactacaagttagttaagagcctatactctagaatcagaatacatagatccatgttctgattccatcatttgttagctgagtgagatatagcaatttacttaaacttagtttcataattcataaaatggtgataaaatatttgcctataggtttcttgtgatgctcaaatgagatattataacacaaagcactttgaatagttacttataattgtttttttcctattctacaataatatacacaactttggaataaggaaaagcagtggacatttttttaaaggcttgattattgcaatgtcacctgaacctggaagacttgtttttctgggtgaagaaatattttctctgtgtcagagtttcacaatcatcgtcaggtaggcccttgaaccctgccatttaaagtgctcctctctttgatctgtggcactcatacatacactctggcaatgaggacactgagctcgcttctgaaatttgacaagataaccactaaaatctctttgaattctatgttgttgtgatcccatggctacagaggatcaggagttgacatagatactctttggatttcataccatgtggaggctttcttacttccacgtgaccttgactgagttttgaataggtaagtgaaggagaaggaggcactcaaagaagtcagccacagaaccagtgtgagaaataggaaatgagcttttttaagttttgcaaaggcagatcaggagagttgacctgtggagtggagagtagtcataattttaaaaaatggccatggaaattaaaactgatcagaaatggctgggcacagtggctcacgcctgtaatcctagcactttgggaggccgagttgggcagatcacaaggtcaggagatagagaccgtcctgactaacacggtgaaaccccgtctctattaaaaatacaaaaacttagctgggcgtggtggtgggcacctgtagtcccagctactcgggaggctgaggcaggagaatggcgtgaacctgggaggcagagcttgcagtgagccgagatcgcgccactgtgctccagcctgggcgacagaccgagactcagtctcaaaaaaaaaaaaaaaaaaaaaactgatcagaaatatgaattccataaagacaagggcaaagtcatgtatttggaagggaaaatgttgcccaagtagagagtttctgggaatatgatcttgaaataaaaataaatgtgagataacctattaatgaaattgtctgaaaaccatacaaacaccaacattatcttcatgatccctagttctagacctctttggtcactgtaaaattataacattttccgtgtatctttaacagctttctaggaaaatattaaccaaaagtaccggttttgatttggccataaagtgacaggagaggtaaggttctctaggattaaagaataacgtattcttatttgattcactttaaaaaattattctaaaatctgttacatatctgtcctctccaggatgaactttatattggctcagcagattgtttactgtgttcttcttctttttctttttttggtctttcctgctcagcgcccaacccaagttcaaaggctgataagagagaaaatctcatgaggaggttttagtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatggtgagttctcatggctctattgggctatttgttgcctcttaaagattagattactgcccgtgaaactgaaaagggaaatcaaaaaaaatgctctgagttgtgagattggatgtttgcctccatatccctgattttggagtcccagataactaaacttaattgaccctggggttcattcagaaaattgttacaaaataattcactggtctcaatccagatgcttggaaacaagagaatattctttataaagttaccaccataattctcatcacagtcaggatttcaaagcatttcatggaaatgccataagttattgagttaataatttcctttagcctacaatatcaataggaatatctaaagattctctacaaatgatatattaactgaaaaatgcaactggaggcttagtgaaagaactagtaattaagctaatcagggcttgggtgaggctggacttgggaattcctttcctctttgtcacagacctccaagggactccaaaaggtcacagaatccaggcagagccctccaaaacaaaaacaaaaacaaacaacaaaaaaactatgattaaaccagttctgacaaatatcagaatgctctgggaaagccagatgctttaacaagtgcaaggatttaggaatttcttctctcacagatcccaaaacagtatccttaagtggtgatccttatgtcaagctgtgagaatatacctcaaaataatagaaggcatccaaacttgatcagtatagtgtcataaagctgctgatgtagaagtgtggagaagtccatcagatagagatatggaaaaaaaagcttggttaaaacaggtttccaaagttctctccctttctctgccattgtggtatgtgtggttgaccatttctcatcatgttttcttacaagactttcaggatcaagcaattcctcgccaaaaaacaaaatcaaggacacgttgtagctatttcttggtgagttaccagaagctgtgaagagagagcatgaggcccatacttttttttttttttgaagggtctcgctctgttgcccaggctggaatggagtggcatgatcatgattcattgcagcttgacctcctgggctcaaatgatcctctcgcatcatttaacatttttttttgtagagatgagatctcactttgtctcccaggctggtcttgaactcctgggctcaagtgatcctcttgcctcaggctcccaaagtgctgggatgacaggcatgagccactgcacctagcctaccacacttactctttgcgtggtctctagagcttcttcagcccccctgctgccagggtccctagggtcttgggcatgccccatttctgaacctattcctgtctccagggatagagtcacagtgaactcagaccagggtaattctaaactgctattctgtatatacccaaggaaatttcatccctgtctctaatgagtctgtcccatgccccttaatacacatgaacacctacacacaggtacaagtcggggaagatcactgtgcagcacataaaataaaattcaggggcgagcactttttttcccatgagataaagttaggaaaaaataaaaatgttcatacagattaaaaattttaaatgttaaaatattaaacccccaatctaaaataaagaaaaaataatttcttaaatcctaagcttgagttatggaattcacattcttataatttacattacctattttgttaaatggaagacttttttttttaatatgcaagtacctgggcatatttagaaagtaaccaaggcagcttttgtctttgaacctgatgcggttttgacagctctatctggaaattgagccagcatatgggagccaagggtgaaagttttcctgggagatttgggctccaaggtactggatagatgctaaagattccattcagtttttcattttctgtcttcatggaactctcacacctacaggggaagaaatggtttagaggttactaaaagtatatagcctggtttaggtgatagtgagctatagaaaaagagaaaataagttgtcaatactttaaatctcaaacaacttcttacaaaaggattttaatagtttaaatacaattatcacaagttgggtaaagacaaccagacctatggcctgataaatcatgtatttaatttatgcatatagttagggccaaaaagagcagagtatctttttgaatgttttaaaatataatctaggattttatgcctacagtccttgaaattacaaaggatctctctcaagttgggaatatgctatccattgcagttacggatgggatttagagactgttttaatttcttgtgattatctcaaacaacgtgttccttactcccaaaactcccttgtatggctacagaaactgttgcagttttagttgcttctcccctgcctttgcccagaggtagtgtggaatgccaccaattttatctgtaatggttcctttcaagcctggggctatgtaagctgctatatatgcttcattccattcatcattcagtctaaatatagctatcaaccactaaattatctgataggtatgcatgaactgactgattctttgcataactactaagctatctgttttatttaggcttgggagccctagggtaaggatacggaataaattgtaaatgatattaaaagaatagcttggcaaggaataatacctcctcaggggccaaaatgcaaggcaacacaaagtgatttttttggtgatataattataacacatgtagtatggtagtgataactaaaataaaattggatagcctcaaaattcctacatgtagtatgataaacaatgttctaagactgaggcctttgggaggtagtgagttatttcctatagatagagagtaaaattattcctataatttggtatgaaagttttaccagaaattttgcttcacagtaaatcatctggattgggtgaagccaggtagggattgagaagtgagtcaggtgggttgcatagatgagtttgtgctgcagaccaaactcctcttcccatagctcacttcttcccactcctgcatgttagagagaagactagaatgtgtcaaatacaacctataggataatagttcagagcattagcttcggaagtaaacactaggttgatgccctggttcttctaatttctagctgtgtgacctagggaaagttacctgctgtgtgcctcatttcccttatttgtaaagttgagacaataacaactccctcacaaggttgttgtgaagattaatgaaaaagcagtttctgagaggttgtgagtgttcagtaaagaaagaaattgctttcctttttttttttttttgagacagattctcattctgtcattcaggctggagtgcagtggcgtgatcatggctcactgcgcctcgacttccccaagctcaggcaatcttcccacctcagcctcctgagtagctgggactacaggtgcgagccaccacacctggctagtttttgtattatttgcagagatggggtctcgccttgttgcccaggcttgtctcaaacttctgggctcaagcgatcttcccacctcaacctcccaaagtgctgggattacaggcatgaaccactacacacagcctattttccattttcactgccattattttctctgtctcttgtgtggctcattactgccacttctgtgcaccatttccatcatgtcacatgcgtagtcaaaaccctcagtgactgcctaattctcagagggtaaaagtgaaattccttggcctggaaagtgcaatcatgcctgaagttctacctgtgggagctgtactagttatctattgtgtaaaaagttaccacaagcttagcagattaaaacaacacatatttaatatcccacagtatctgtgggctaggagtccaggcacagttcagctggatcttctgcttctaagtctcatatggttgcattcaaggtgctggctagagctatggtctcttctgaggcttgactggggatggatatacttccaagccaatgcaattactcagaggccgtctttaattatttgccatgtggccttttccatagggtagctcacaacgtggcatcttgctgcaacaagtcagcaaaggagagagtcttctatcaagattaaagttaaaatcttatgtaacgtaaacatgacatcctgtcacctgtgttgtatgctattggttgaaagtaaatcacgggtctgctcatacttatggggaggggatcacacaagggtgtgaccaccaaaggcagagatcatgggagcattcttagagtctgtctgccacagggccaggatcaaatgctggtggctgattgccagctccactgtttatttctctcagccgctttctgtatgtacaaaaaaggagagagagtgattttctacctcatagtgttgttaagaaaatcaaatgaggtaaattattttgcaaagtattctctactcatctgactcctacctactttcaggtctgtcttaagttgcaacttctgcatgagggcttggccaagtatggtcacctgcatgaatttcctctttcttgtaacagaaactgcttggcccactcataatttatacattattatatactcttatgtttcattaatgaaacatttataatgcatatcttatctgtctgataaaatggaaagcaatctaaggacaacgacttttcccctatacataacaaaatataaaataaatcataacataataaataataaataaataataaaataactaacatttgttgaatgattaggatgtgttaggcactcttgtaagcatttcatttttttgttacttcttcaaatccttacaacagtccttggaggtaagtcctattattactttcattttgcagataagacaactgtggctcagagaggctaaggtacttgagtccaggagcagaattcttactttcctcaaacgttatctcttttgtgtggtctagggcagtacaatataccaagtagttgctcattttggtctttggtaaatggaaatgaatttgtaagttgagaaaagactacactacagtagtttggagaagtccactttccaactgatgttcaagattcaggagggggggaaatgtgtcctggcatcatcacttgcttgatgatttcttttgctggtagaaaatcaccatggctaccaatggccattttctgggaagccttttttgggaatcctaacttaagatcaaaatccctgggctaagtcacggcagctaggaggttactgtgaaccggttctcatttcttatggtttccattgttattagggggctgtattctcattaagtccactctgagctctactggattctctttttagagcactcgtctttgaattcttggccaactcatctatgtcacagcactaaggctataaattccagggccccacaatgtggattatctgtgagaattcatttccagttctggggacaagacccttgggaaaaccaggcaataggccagagagagacagagagagagagagtggtcaaaagtggcctggtcactcttaacctaaaccttgaccttcagcggagtagaggactaaatcactcaggaaatattaaatgtttcaccagatattatttggactatatcttctgttctatctcttcaagcaatgccattccaacttcaattttcttttctgtcatttcagaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacgggtacgtttgtgaacattttagcattgatcccaagagttgaaatatttgggaaatattaaacattaatatcacccacagcagctgcctttatttgaaaaaatagtaatgctgatgttagaaaaatctctctctatgcctaggtaaggtgtccatataatttattattcaaactggaatctttgagagtgaaagacactggtaattattatattcaaacacagagttaaactgagattgtccctggcactgtggtgtgtatggacaccccctagtgttaggtcaccttcctgaaggatcaggcagcctgaagaatacagttagtttatgtcagtggttctcaactgggggtgatttagccctccggggatatttgacaatgtctggagacgtttttggttgccacaaatggggatactgttgacatctagtgggtagggatgctgcagaacatttcacaatgcatgggatggcccccaacaacaaagaatgatcttgtccacaatgtcaacagcaaacttttttttctgtaaaggaacagatgacagtaaatattttaggttttgtgtatttaggtattgtctgtgttgcaactagtcaactgtgctgttgtatcaggaaatcagctgtagctaatatataaatgaacaagtatggctgtgttcaaataaaactttacaaaaacaggcagcagacaggatttggtctgcagaacacagtttgcagacctctgatatagcctattgaaatatttttaacctaaaatatttagctatttaaaaagaaagtcttctttctcagttgcttgtcctctggaactttgtatgttctaatctgtgaatataaaaataaaaatgcatcaaaaaggcaaagataaaaccatgtgaaatgtatctttctacaagttaacatttatttttctgcattctttgggtccaattatccaactaattatcaatgcactttactttatccattctatgtttctccattttttcccccaacaaggacactataagaaaccttgcccattcgcatatagacaatctggtctagcctaggtggaacctagaaattctattttttaaaaaatccccagataattctaatgtgtagccaggactgaaaaccactgaaatgcatgtaagagcttaaagtgtttgggaagaggagggactcaaggttgcttagatttgatggagaggaagccgtggagattgaagaagagctggagagagaccatgaggggttttgtgccatggtaaggaatttaggtttgttcctttggactatgggatcgtgtggaagttttaaacataggatctatttcatgttttaggatgactctcaaggcagaaccaaagggaaataggagaaggagaaacagatcctttaatccagtgcttcttaaatcaatctgagggagagaaccatttccttccttccttccttcctttcttctcttcttccttccctccttctctcctccctcccgtctttcctttctccctcctttattcctttatttccaatacattgcagatcaatacttgcttaagttcattgattacatgcttagatgtcatggaaatatcaaattgttctccaaagtttataaacttttattctcttaattcctgtacttatcacagaatggaaacaaactgtcacttgcactggtccacggattacacctggagtagcactgcttcactatatcaccttcactttatctgcagggtaaagtttaaaaactttggtatggcatagacggtccctcgtgatctggcccctgcttgtctctccattctcatgtctttcatttcccctaatgaacaccattgctccagtttcattcaacatctcactgtcccctgagaatgccttgtcttttccaacctccttccatcttttagactcttccctctacctcccttctctacatgaccagtgcctacttcttctccaagacacagctcttatcttcccttctctttggacccattcttgaatctcctggttattagttattaatttctccctgctcctatactaccgcatcactttttggtgacagttatagcacttgtcattctgttatgtattaatagttattgatttcatggctgcctcactgtcctatgactttatggtctttgacttgttcacttcagaattgtcagagtgtaggacatattccaggttcaataaatgctgaatgaaacaaaaaatgcacctctctgtgtatgtgtggataagaaaacttctttgggttttggtagatctggttatttcaattactccatatatatttattatgtgcttatagtactttgtgctagtcgacagtggggaaacttaactgggctattccttctccacatacagaaagatacatggcaaagaagtaaattgctgtattttgagtaatcaaaaaatcatgtggtcaaaaggatatctttatattagcattctcttcagcaaaatttccattgttaacattgtttattatctttaatttgcagttgaacacaattctaaatacaatgagcaccatctacagtactggaaaagtttgtaacccagataatccacaagaatgcttattacttgaaccaggtaggctactaatttttagtagtgattatgaaatttacttttctctcagattttaaaaatgattgcagatatgtgtgtttcaacacatagatgctcttttaacttttagtaaaactttcttgtaaattgtatcacatttacttaatttgatcaagtcagtaagtaagtcatttcagtggtttattttctctgcagacatattttggaactataagggcaaggacatgaatgaatcatgagattttatttaaccactttatgacctgatgtctgccaaccttacttttaattctttaaagttcatgtagttctttacaaataaaatttttacaaaggcatcttttcttgtctttgtgtgctttgggataacaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcgtaagtttgctgatctgtagaggtcctgaagctctctgtgtggccacggggctatttcttctgcattaccaagcctgccctttgatatcagggatacccctcaattaacaatcatgtcctcaacaattcagattacctgttttcaaaagaaattactggggaaaaagaaaaggtgcactggcattctacaattcaagaagctcaagaatgagactcaactacaaggcctaaagtaaattatccaggaattatgaattataagtaatctaggaagatctcaggcccctaaaagttcccgagggagtctggggattaagggggggaatcccatttctcaggatgaaagacagagtcactgccttttgtctaaagcccccacagttcactttcaaaggtcatttgtgtgtctaccttagggtgataccccagaaagaaaaaatatcagattagtttaggctggaaatgtattattttctgcagttgtaattatatctcagattatgtggtcataaaaaatcttaacataggaaggaatttccatatctcttaacttattctcttccctgaatagtgagctgaagtcatggaatcatgattcacattttgctttgatgatttactgcccactgaggttgagtaaggaagaatttttttcccgcatttgttgttttaacatttctttcactgccacttaaattcagtattcttaagccttaggcaagctggagacaatagcttatcaccatttttaaaacaatatcctgcatgtcctttcatccaaatatttatttctctcctcatcttcctgatcattaattgccaatgtgaatgtaaaaataaaaattattaaattatgtttacaaactttattagctaatatcctgacacaagcttgtgttttctgatggtgagatactaaaggaagcacatagaaacatgctattgatacatataatttgcaaacaaacacaggttcactgtggcaaagtctaagatgagttgggtctgatctaaaaataaaaggccatgaacaaaagaagaatttaaaaacttagagatccagtcacatattttaaagctaaagatacaaactgatgtatcaagagagtacacacccagggttaagagactaggaatgtgcttctttcgcttaccagctgtgtgatcttggacaagtgatttaaccactctaagcacaggttgttggtgggaatataaactggtgaagctgttatggaaaaatagtatgaggattcctaaataaattaaaaatagaattaccgtatgatccagcaatccctcttctgggtatatacctaaattagatgaaattaccacttcataaagatatctacactgccatgttcactgcagcattattcgcaatagccaagatagggaaacaacctaagtgtttgtcaagggatgaatgaataaagaaaccgtggtgggtatatggaatgggatattattcagccttaaaaaagaggagatcctgtcatttgcctcaacatggatagacctggaggacattatgctaagtgaaataatccagacataaaaagaaaaatatggcatcatctcactcaaaagtggattctacaaaaaaatcaaatatacagagatagagtaatagtgattaccaggggtgaggtggagcggggtgggatatggggagaagtgggttaaaggatacaaagtagcagatatgcaggatgaacaagtctaaagatctaatgtacaacataaggactgtagttagtaataatgttttgtattcaggatttttgcaaaatgagtagattttagctgctcttccatgggggggataatgcttaactatgtgagatgatggatatgttaatttgttttactatagtaaccattttattatataagtatctcataacatcatgttgtacaccttaaatatacacaataaaatttatttaaaaataaacataggctgttactctgcacagtgggagttataataatgacaatcttgtatggttcttgtgcttattaaataaaataatacatttaaactcctggcacagagcaagtgcttaaacagagattacttaatgttattattcatttgcatccctttgagttgatgtttatcacaataaaaatgtgataagcctttaaatttcttttaacctcccagcaaggctaatctatgtattttgtgctttctctctttctttcccttatgttcttccctttctctttaaaaaaaaaaacaaacagattatgaggactatggggattattggagaggagactatgaagtaaatggggtagatggctatgactacagccgcggccagttgattgaagatgtggaacatacctttgaagaggtaagcaaggaactgtacacacaaacagttgtggggcagtgtatgggaaagagggacagtgactaaaggcattctggctcattcttaacacattggtcagccattcccaggcctcataaagttatggaatattagagtttgaaagattttagagaataataggacagcccctacagatgatcagaattgcgctgctttcctccttctttcagtcactgaccattaattaccaatagaaagactataagaatgaaatattataagaacaaatggtcctttagattataagtaggaatgatcttcttgagctacctggggctggcactgtcttcctgtgttagcagagctatggactttgctttcttcctcaggacaattgatactattactttgagtaagctagtcttcatagggaagagagagttttgtgaacagaaatgtcccatggatgtttttccttcattttggccccatgcctcccctttacgtgaaccttcaaacttctgatgtgagttaccttgtcacagccatagtttggcagagctatcaaaaatggaagtaagacaaacaactccccaaaaaggaaatgggaagaagacttgaataagtacatcatcaaagaaattacacaaatggcaaataggcaaataaaaagttattagggaaatgcaaattaaagctatgatgaaaatccactccacacccaccaggatagctatgattaaaaagatggacaataccaagtattggggagaatgtggagacactggagccctcttacagtgcaggaagaaatgtaaaatggggcagtcactttggaaaacagtttggcgatttcttaaaaagttaaacacacacttaccatgactcttaggtttctatatgaaataaatgaaaacatgtgtccttatccatagaaagacatacacaaatgttcatagcagcaatattcataatagccccaaactggaaaacccagctgtccttcaactagtgaatgaataaacagactctgatatactcaaagtagccggagataggagtggaactgactacaagaaaccttttgtggttatagaaatattaaaaaactagatagtggtgatggtttcccggtagttttaatttattgaaactcatggaactacacacttgaaaatgggtggcttttatagtatgtaaattatacctcaatgtaaaaacaaaggaagtaggtaacaatcttaaaaatgaatatatattttttgcatctgcttttattttgggacctgtgttctcccaagtatgtttattggtttatgcagtggagttatcatcaagttctaggaagaaggaatacttttaaattttccagaaatttgagagaagaagaattataatttggggaaattctacttgtttaaagaatctcctatttctagtgttgtggaatggaaattagaattggttactctttgtcttaaactttcttcctgggcttttcagattaaaccattatatgaacatcttcatgcctatgtgagggcaaagttgatgaatgcctatccttcctatatcagtccaattggatgcctccctgctcatttgcttggtaagaagccccatgaattcttcgtgtacttggctctttgttatttctaacaataaaattgcctttttgagtgagccttaacataaaacttttgaaaaattctgtaagtggttagtaacattgtagaaatcgcattttgaaagcattttcttcaaccaaaagtctattttaaacaattctctattgtattactttttttgtacataaagagttattattttggctgcaattcaaatgataaaggggttatttaagataactagagaaagaattacaggctcaaatgagagtggaaatggggaaaataatttttagtattcaagttgattggctaagctcttaaggtggaatttgagattttagtatcatattgtcagagcttggttggctgggggtggtcaggggtaacagaaggttatagcttgagcgaaagcttcctaagttcattctagacttcaccaattactcttgctgaaaagtgcttggcttggaatcgtataagtgaagaatgaatgaaagtgaccatcttcccctctcccaccgcagcagctgaggccaaataaaatataaatatttgaaaatttcatgccatgcatatatagcatttctactgtagtcaaactaagttgctggaagctaatctcctccacagccatacctcgtgtctgtcagcaggagagaatgcggagaaaggacctgtggaggcttttaactctaaagtaatatggaaatccttcctacaatgtcaatggcaagcattatttagtaattcagtttctgcttaaacctgtctggtgtgatgggataatttcttcattccaatatagactaatttactgctacatggaaccaatgtaatacaattctaccttatgtggaacttaagcaacacccctctgatgccttccattcattagccctatttccatgctatgaggttacccagaatgtcaattcctattctacaagatgaccttccagatattcataaagaaccatcatataagatacagcatagtacctggtgcaatacattttagtgactattattgttattgtcatgtttaccctattttcttttgtcaagttaaatatcttatacattatcaaccattccttaaaacctcacttttctataattgttctcctttcaatgagctgtactttgtcaatgtccctcctcaattatcatgcttagagatgaacagttactccagatgaggtttgaccaacataaagaaagacatggtaccatgactacctttgttctagacactatacttctattgatttagctcaagttcacatttatgctttagaagccaggtcatactgtttgttggctcatttttggtcttttacatgtggactgtgctttagcctgatgtcacatatatgtttttatgatggataggtttttctttaaaatttaagcataggacttcacatgtatcactactaaattttatgttttaaaatataagtttagggagtacttttatttttttcagagtctcgctctgtcgcctaggctggagtgcagtggctgatctcagctcaccgcaacctctgcctcctgggttcaagcgattctcctgcttcagcctcctgagtagctgggattacaggctcaagccgccatgcccagctaatttttgtatttttagtagagacagggcttcaccatgttggccaggctggtctcgaactcctgatctcaagtgatctgcctgcttcagctctgcaaagtgctgggattacaggagtactcttcatgaagtttgttgccccatttttttggcaccaggattctggtcctgctcttggattgactttttctagatacatcttcttttttatggtccctggccttggaatacaacttaacatttgtttgagatgtttacaaagtcaccaagttaagtacacgaatataggaataaaagtggaatttagttaccagttagtagaagttcctgaatgatgatcttatatgatctcttttgaagccaacactaggaattactaacagcttaatttgtaatattttgtaccatggatggtagattatagaaatctctttctaatttacagtgtcaatgatggtccatatatttaataaattatgtctacatttctgctgtgttgtcatatactaacagattcttttttaaatataggtgatatgtggggtagattttggacaaatctgtactctttgacagttccctttggacagaaaccaaacatagatgttactgatgcaatggtggaccaggtaggaaaaagagcccttaaaaactaaatctaaattctcaacttcatttatttttatgtcatccttctatttttatttttatgtgaaaccatgtaaataaaaaatttaagctaattgaaatttatttcttatgtagctatagtttcatggaaaagcagtttacaatggcctcagataacttgaattgcaggtgtagtattgatctcattgaaaggtatttgaaatggagattacctgagttttccattaagcggtaagatgtagaattgtcaactggttggtatcactcattccaaagtttgtcattgctaagtaaccagccagtaatctctatggatagtaaaaacaagaatattatgaggggatttttgcatatttaaaaaattattagtgttaaaaatattacaagaatctttaaaacaattttaagtaacatctttattactactttggtttaattatttaaatgtgctaggttcctcaaaacacaagtaagtacatagcccatggacccaataaagcaactacacaatcgagactacaaagcaaccagctaacaacagcatgacaggaacgcttcacctaacagacagagagtggcaaattggagagaaaaacaaaacctaaccttctactgtcttcgagattcatctcacgtgtaatgacacccatgggctcaaagtaaagtgatgcagaaagatctatcatgcaaatagaaaacaaaaaagagcagggggctgctattcttgtatcagataaaacagactttaaaccaacaacagtaaaaaaggacaaagagggtcattacataatgataaagggttcaatttaacaagaagacttaattatcctaaatatatatgcacccaacattggagtatccagattcataaacatagcacttttagacctaggaaaagacttagacatccagaaaataatagtgggggacttcatcaccccactgacagtgttatacagatcattgaggcagaaaactaacaaagaaattctggaaattctggacttaaacttgacacttgaccaattgaacctaagagacatctatagaatattccacccaacagctatggaatatacattcttctcatctgtatacgaaacatactctaagactggcaacatgctcagtcataaagcaagtcttaataaattcaaaaaattaaaattatgccaaacatattctcagaccacagtagaataaaaatagaaatcaataccaggaggaactctcaaaaccatgaaataacctggaaactgaacaacttattgctgaatgacttttgagtaaacaatgaaattaaggcagatatcaaaaaattctttgaaacaaatgaaaacagagacacagcataccaacaacatatctgggatgtggcaaaagcaatgttaagaggaatgtttatagtgctaaatgcctacatcaagaacctagaaagatatcaaattaataatctaacaccacacttaaaggaactagaaaaacaagaacaaactagttccaaagctagaaaaaaagagataactaaaatcagggcagaattaatgaaattgagacctaaaaaatcatacaaaggatcaacaaaatgaaaagtcagttttttaaaaggataaacaagatggaaagacctctagctagattaacagagaaagagagaagatccaaataagcacaatcagaaatgacaaaggtgacattgcaactgatcccacagaagtacaaaccttcctattgagtactatgttcatatctggttgataggaccaatagaaacccaacgtcagtatcacacaatatacccttgtaacaaacctgcacatgtactccctgaatttaaaattaaaattaaaattaaaattaaaaaattatcaggccgggcacggtggctcacagctgtaatcccagcactttgggaggccgaggtgggtggatcacgaggtcaggagatcgagaccagcctggccaacatggtgaaaccccgtctctactaaaaatacaaaaaaaaaaaaaaaattagctgggcatagtggtgtgcgcctgtagtcccagctatttaggaggctgaggcaggagaatcacttgaactcgggaggcagaggttgcagtgagccgagatcatgccactgcactccagcctgggtgacaaagtgagactctgtctcaaaaaaaaaaattctctgtgttcccttctgttgatgaaatggtattatagttataattatgtatatttgttcattcaactaaaattctacgtttctgagtagaatatctaatcatatttctatttgccttataaaatttcttcagataattgcaccataatttactatttgtgcattgctgaacctttgcttgttaatagttttacatctgtaagaagcaatgcaatgaacatcttcacatacataacatactcctgggaaaagtccactgcagaataaaaagtgaaactgcaagggcctctaaactcttgtagctgttgtagctccactttgaagttgagcagcagtatagggccatcttgatgtagatttttagtctttctaaagggtttttacacccgtctcttctattgtcatctctgactcaggataggtattgtgacagctccatgctataggtgaggaaactgaggctcaggtaaatgatttgcccaaagtcacaaatcacacagcctcattctttctgttttttttgtaccaattaaccatcctcacctgcccctgccaccttcccactactctttccagcctctggtaaccatccttccactctgtatgtccatgagttcaactcttttgatttttagatgccataaataagtgagaacaagtaatgtttgtctttctgtgcctggcttatttcacttaatataatgaccttcggttccatctacgttgttgcaaatggcaggatttcgtttttttatggctgaatagtattccaatttgtatgtgtaccacattttctttatccattcatctactgatggacacttaggttgtttccaaatcttggttattgtgaatagtgctgcaacaaacatgggagtgcagatatctcttcaaaatactgatttcctttcttttgggtatatacccagcagtggaattgttggatcatatggtagctctatttttacttttttgaggaacctccaaactgttttccatagtggttgtactaacttacattccttccaacagtgtaggagggttcccttttctccacaccctcatcagcatttgttattgcctgtcttttggatataaaccaaaaaatttttcataaatataatttgggtggctgcacattattctatttttcaaaggtatcattatttatttaaccattctgatgttcgtttcaactttttctctattaaaaataattctcaatgtaattgttgaatctaatttctgataattttttaggttagttccttagagacagcattactgagacaaagagaagaatttcaaatgctatccgtgtgtgttatcaaattaccttttggaaaggttatgccaatgtatatatccaacagaaataatgctgctattttaaaaatgacattagcacttttttccattttaaaattttattgtaaattgacatttattattgtatatatttatggggtacaaggtgatttcatgatttatgaatacaatgaaaaataaataaagctaattaacatatcaattacttcacatactttttttgcagtgagaacatttgaaatttactctcttagcaatttcaaaatgtacaatgttctattattaactatatttactatgctgtgcagtagatctcaaaaaaataaaaccaacttattcatcctgtttgagactttgtacgctttgaccatcatctccccgttgcccccaccccatagcctctgtaaccaccattctactctctgcttctaccagttccattgttttagattccacacataagtgagaacatgtggtatttgtttttcggtgcctggcttatttaatttaagaatgctgttgttttgatgctggaattaatactgttctcttttcccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggtagtggggctgatacttacacaactgatactaaatgggagacaacagaggcaggctgaagtttaacttgaattctttctctgcttttctgagcacagaaaagataagttaattctccttgaactaaggctgggagtccaggagagcacatttgggttttcccaggaaaagaggattgccagcaggctctaacttgaacctgtggacaatctgctttcttggaaatatcatgcatccattggcgttgtatggtaatggggatggttgcacccatatcaggaaagatttggtgctaagatttttggaagatggacatatgaatcacacattgatcttcccctccatgtttcactcttgtgaatgagacagaataggtgaataaatacgcttggatcgaagggagaaagtggtacatgtcgtaagagagacacagggcatgctctatggagaactggaagaaaactgaccacatttgcaataggagataggatcagaccgtgctttacaagtgggatttgaattaggtttggaaagacaagaaggattcagatacacagagtcgggaggaggacccaagctgtgagaacagcaggatcaaatacagagaggcaggacctgacctgcatactgaagtcggcaagttaggctagaatgagaaataagtgaaggagagtttgtgtaatgtggcagaatgagcacaggcttcagaatcctaggtgtgtcacttaatgactatgcaaccttggacaaggtatttaactttctttggtttcagtttccttattttataaagtagaatagtaattcccaggttgcaggcttgtgagagccttaggttggattccctagcttgaaaaggagatcgttttacaagtgcttcattgaggagagctctgaggcagaggggaatgagggaagcaggctgggacaaaggagggaggtaagtaagaatgtgatctttactggaaacttggcctcagccttatcccatgaagctctctggagcataacttgagccacagactaggccacacctcaagacaagggggctagccttttatatcaggcagtctttggctgtggactgccccagagtatgggggagcataacccccatttagccaaaggcagtgaagggagcagctgtaggcccttatcagctgtgactcaggcagctaggggatggtggataaagtcttggcaggcccgcaccagcattgactagggtagggattggatctacagtccttttctgtaaagggccagatagtaaatagtttaagctttgcagatctctgttacaactactcagctctgcctttgtgacatgaaaacagccatagacaatacacaaaccaatggatgtggctgtgtgccaataaaaatttatttataaaaacaatggctggccagcaggccatagtttactgatgccagtactagagtgaggcttaaaaatgagaaagtataagtaagttttcaagcacagaatcttacatacagtaggcggtaaatatatagtagttatttttataattatttcaaaagagaatagagagaaaccaaattagaaaagtaggttggggttgtgaaaggaaaatagatttactgagcatgagatgaatacataactgaatatttccttaccttccccctttctcttgcaatgtgtgaattaccatgccctcccactatcctctccagctcacttttctcctataaatgttgaagccctcaaaattatttttggagaaaggcacaaaccacagactgtttctataaattctgtgttcttttcttctgggcatgttcttaaccttggcaaaataaacttgtgaattgattgagacctatctcaaatagtttttggtttacagggtcatattggtgtataaaaggtggctgagtttattcttgagcaatgaggaggcctcgcaggatattggagggggtggtgacatgatccaagtggtatgaaatacatttaggaatctactgatgtatgacccctacacccatctgaatgaaaatgctcttgctaagttaaaaaggaccaccatctcattgccaaatgcagttcttatcccactgggctatagcagcattagacaagatttacaagttgtctttcaaatgctttttttttttaaccgacttccacattgtcattcttttccagatttccttgacctctctgattgtactttctccgtctcccctgaataatcttcctctccccactccttaggtacttggaccttctaaaggtttatctttggtcctcttttcttgcattctattcctgttccatggcccatcatatctatttcccaacttacatgctaatgattcccaagtttcaatttgcagccccttctactatttctctatcctggcaataccaaacagcgtgcagtgtcctgaatgccacatttccacccccctgcccctaagcctttctcattgagcactagaacgtcaacttccatgtcatctaccgggcaaactcctgcacattgattaaggtccaaattgctcattgtatcctcagtcatgcccttcttggtgcttctagggaactgtgcctacattcactcatttttacgctcaaggctgtgtgtactgatcacttatagagtacttgcctcagagttcttgtagcggagaactctgcttatctgtcttcctgttagtctatgagcaagagaacaggattcagctgtgcccattgctttatattgagcaactagtattgtctctaggacataagaggtgggtactcaagattcactggtgaataaatcttggaaagatgattcatgctaccaaaaccccatacaactccactgtaatggttaaatgaaagtcagtgagattcattttcatcttgtccattttcatgcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatggtatggacattttctcatggcttgttttggaggttctttaatctgatatgggaaaaggtatttattatgtaggaaatatttactttttgccacataaggtatgaggtatttatgggtaactggaaattactactggtgaaaaaatttaagtatatatcagaacatgtcaacttttggttttgtattattgaaaagggaataagagaggtaactgaatgaatggacttgggatcctgtgaaatatctaacagaagtttttttcttaaagatgatgaaacagtctcacagaactgaaatggcttgtccaattagtgtcagcattctaactagacccctctggttctaggtaaaaacttcccaaaactttagagttggaatatattctaaaaaccattttatagaggagtaaactgaagacaagagagggaaagtagcccgaagtcaaaacgcaatgtagtggcggaaccagaacaagagccctaatctaaagtcttttccataatattatgcaaagcaaagtgtagctttaaattttggaattcttaaagaacaggtgtttaatatcaagattagaagggattgaaacattttgttcatcctgctgaagcgtgaatttcccctgtaacatccccttagatctgtgctatccttcaacatttctgcctgaggcagcccatttccttttagatgccttggacttgatggaacatgacaaatgttcaggagatccaatttcttcagtgaggatgccatggcttttgattcttcctcataaatggaggacaaatagagcctaataccttcttctcttctgtatattctctcccctatttttagttatttgtgacataaagatcagctcctctgtctggagcatttggtcgatgaggtcaaggtcatgagctccatccttgagtagacaattcactttgccttgatctctggttccagatacttggtgatcccacaagggcctgggagtgagccagtgtaaagcctatcatcatgtttgaaatagttactgtaagcacagtcctagtggattacagaccagtagcagcatcctcccatgaagacagtaacaaacaaaggtagttaacagtaaccatacctgcttcctaattttaaaatatcattaattcaagtcaatgaacagatattttattatttttaaatattccctgacagcaggtaaaaatggttgatgactatgtttcttaagatgatattagatcagtgatgaaataatctgtacaacaaaccccatgacacgagtttacctatataataaacctggacacatacccctaaacctaaaataaaagctttttttgtttttttttataaaaaagatagtattagacatggttaaaccaccggtttggaaaactcctgctttttttcttgactctgcccctgatttgtgaataatgttaaataaattcctttattccttccattcctccctccctctttctctttctttcttcttccaaacatatttatcgagcacatactgtgtgcaagacatttttctagacactggagacacagtgatgtgaaaagtagataacatcccctgcttacatttcaatgcatttccttcatacactagagatgaactccactgcctctttatgtcactaataaaggataaaatgaaataacatgatttgttaaagcattttaaattcttggtaaggaagatatccttacatttaaagtacattgtacttttcaccatccagtggatagtgccctccaggagggctcttttgtttcatttcccctccctggtttacaggcttttcactgaatctgtttagtatttgtttatttaacaaagactcacagaaggcatcacgtagcagacatgggactaagttatacagtcttaaatgagacattaacagtcactgacttcatggagctttccagaataaacagtgtgactgggaaagtggatgaaatagctctaatgaactctgataagaggtatttaagggaggaaactgaaactaatgaagaaaatgaggattagaagtaactttgggttttatttacctgttctgttccagggcttcaacctactccaaatcccttagctgagaaaataaatatatacatatcattatgaatactgtcaatttttctcatgtatccttatagtaagtacatcttgaggaataatattttgaggttaggaaaattctctttaacacaaaacatccactgtcatcttcatcgtaatatttatccttttctattttactttcagaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagtaagtcaatgaatatgcaatcagtaaaatgttttctaatgagtttgctgtgtatttaaaagcatttgactggcgtgtacacacacacacagaggcacacattaactaactactttttgtatttttagtttgttatattttatattttagtctccatttggataccatctcctcagataaaatttccttgggtcaggtgacactacttcatactctatattcttaagtattggcttactcattaaactatgatattatgttgaaagtgatcaatttattcattttacatattcttttatccagccttttaaacaaaacggtgaccccacattttaggcaaagcactggggatccaatgttaataatttcaggtatggtcctcaaccccaaaaaacgtttagtttatggcaggatgtggaaaattaaaccactgcaatttgtagtgttgtgatgggagggtacagggtccgttagcgtccaggggagatccaaagcccaactgggggtgtcagatgaggagaggcatttaagccctgagaaaggtgtgggtgtagaggggaacacagagaattccaagctagagagaacctgacacatttgggcaacagtgaccatttccatggttgcagcactacgttacttattgctgtgtcacaagtgcctcacactgtgcctgtcacatgcaagtatgaaataaataggtttttggaggaaggaattataagagaaaaagaaaaagaaggaaaaaaagaaaggagagagggagggagagaatggaaacaagaaagaaatctatcttctttttaccatcatagaccctggtcaattatttctttctttatttttcttatattttctttttgaagtgaagaactcatgtgtaacaagcagttccacatgcacaggtttaaaacagtacacttcttctgtcaagattctctctgttaacataaatcgtacttgggaggaaagacgcagcacctataagaactgttgccagtgtttaaatattactgaagttatgtctacaatgatcctgcaattttaaaggaaaaatgtattccatttaatcaaagccaaatacatgattttgctacctttttttctggttggattaatcttttgacccctcttagcttattttaaaggcacatggaatcagatccttagagtgattcattaatgcatcaattgttcctttgttcagtaaatataaattaattctttctgtagtgtcaggcaccatgtttgacactggaattagagagaagaccaagacacagccccaccctctaggagctcaagtctctagggcaggaagttactatagttgaataactacaaaacggggagataaatggcaaaggaggcacaactagagaaggctaaaggagattgaggggttaattctgccagaaggtggatgggatcagggccaggcttcacagaggagggaacacttgagctaggtaggcgtggagaataagtaagatttcaattttattaaattctctctgccaggagtacatgaagtgtggtgcctgagaggaattattagggctttcactgtacatttttggccatgcaatgcactttatgaatgttattaaattagtcttttgaagacatagctattttccaagggtatattctggctgaattaattaaaactaagagataactacataatgagctttactgtctgattttttaaaaatttttatttattttttatttttgtaggtgatttttttttacattgtttctatttcacccaacaaccatttctaatgtaaataccattgattggctggacacggtggcacctgtaattccagcattttgggaggccgaggtgggcagatcacttgaggccaggagttcgagaccagcctggccaacatggtgaaaccctgtctctattaaaaatacacaaattagttagatgtggtggtgcacacctataaaccaagctacttgggaggctgagatgggagaatcgcttgaacctgggaggcggaggttgcagtgagccaagatcgtgccactgcactacagcctgggtgacggagtgagaccctgtctcaaaaaaaaaaaaaaaaaatttgattgatgaaactgcactagttatgcccacctgcttgtgatgcttgtgatgggtgatccacagctaatgtattgttttcttttctccccaaaggcgagagatagttggggtggtggaacctgtgccccatgatgaaacatactgtgaccccgcatctctgttccatgtttctaatgattactcattcattcggtaaattacagttttcttgtttctgtttaacttctagggtttgtggacagctgtgctaataacgacaaaaagtagggataaatgaagtgataattataatttttctatttccacccataactaccaagctcaatacagatgctccttgctttacaatggggttacatcccaataaactcatcataagttgaaaataccttaagaatgcgctgaatacacctaacctatggtacaccaaagcatagcctagtctaccctaaacatgctcagaacactcacattagcctactgttgggcaaaatcatctaacacaaaccttattttataataaagtgttcaatatctcatgtaattatttaatactgtgctgaaagtgaaaaactttgggtacttaccattaacatatacagctgaaagcgctattgtaaagtcgaaaaatcaaaagtccaaccgttgtaagttgggaactgtctgtatagatgatcaatttcaattcaatttaaactaacacaatttattgggttaaattaaacttgtgtaagatcttgtccccgtcatcagcaatcaatagtatcattgggagaaataaaaagtagttttgtcttcttgttactggcagtttattgtacattgtgatagagaatttttaacttgaagtccaaaaacatatgttcttcacctagtaaccccagtccttgaatttgctggagctcagtttatttgtaaaatgaaaataatattatcgacctgttataaggatggatttgcatcatttatgtgaaagggctattaatctgtaaaacacaaaacaaatgttagctaacatttaacagagtaatattgccatgtttatcaacagatacataaaacacaaataggttatgatgagtgtagagttcatgtctggagaaagagatttcagagatttgatcagtatctctttggttacttgggctccagatttaaatatatgccctaatctataactctaagacagtaagtaaacacgggactgcttttttgttgctttgtctcctgtgcagatattacacaaggaccctttaccaattccagtttcaagaagcactttgtcaagcagctaaacatgaaggccctctgcacaaatgtgacatctcaaactctacagaagctggacagaaactgttgtaagaaatacctcaaaatgttgaacctctcctagtattcagtattactcatttccatgcctaggtttgtatttgatttctttgttctaaaaagaaaattttatggcctcaaaatgtcctcatttacaaaccaaacatttaatttgtggtcagacaggaacctagaccatacaacaattgggtgggccacctcttttctccctatcataactacagccctctcttcctggtaattggaaggaaagagcggtttagggtggaatatatctgttaatatgcattcttttcttatctgccagaagcaaatttagccaagtcaaagagaagaaaccatagatcatagatgtaaatatatgtacatctggaacccctcaaaaggccctgaaccccctttttttgtgtagcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatgtgagtacacccagttgacaagtttcacacccattccttgtctacttcgtctagtctcctttggccccagtctcatgaggacttgtgacacagctgagaatgtttctttctctatagtacctgcttctttctctgctatggtgacagctgccattttatgggagaaaaatcacaactgttgagcacctatgtatgagacatagtgaaagcaactgtacagacaaacacacacagatgcatgcacatgtgtgcatacacatacaagagtgtgtactctttcaagtctgttttattagtctcatctttaaaatacatattataactgtctccaaccaactgtactcgaatcctgctttccaaagacaaatactttttttaaaacaattttaatgctattatctcttctttctggcagtcactttcatatttctaaataattttcttatattgctattttgttattgctcagttttaaaccttcgttttggtctactcttccacttccatttcttgctcttccaataaggccttataatgatttttagtaaaacagttatcaatgtttacatcactatgcctgaagagccaagtaatgtattattttgtgtcctttcttatgtggcttgttgtttgtctcaaagtttctacttgcctgaatttttcggcttacctaatttgctatctacgttgtatttgtttttctcccaaatatttcaacatatctattcttttcagtctcattttttttccctagagacttccctcttggagtcttatattctcctttcccaacatttgttgttctcctgggttgaatccactgcttcctagattccatgtcttctttcttggattacctcttgtttttctggagcatagcctcccacagactgtccaagcaggggcacaaggggtaggaggatcaactgttctccaaagtggttttaaccacaccctgacttccattcctcttcttttcagcccagtattattttcccaccctcacctgtgcttgaattccaaacctcttttggattttttggctctttgtgggtctgtgcctcctctgtgtacaaattaggtcatggcttcctgtccctgagaaagcagttacccctgtctcatcatttctcgttttccaaaagcctgttgatacctttaaaagatccctttactatcactttagtgggatctttggaggaaaaggaaataagcacatgtgggcaatctgttgtctgtaaccagcagtctgctatttctctttaatcagatgcagaccaaagcatcaaagtgaggataagcctaaaatcagctcttggagataaagcagtgagtattctggacagtgaattgattatttttgagtgcacagtcttcacttaaataggacacaataaacccatttcagtgtgaactgaaagaagagaggccatgtggttcttttcaaaactataagttccagattctggcctttggctttgggtttttaaagtaacctattccttattggataagctattcctacttcttattgccttgaaggtgttatgaacttcaatgagaaaatatctgtatcaatccttaaatatcagataccaatcctttcaattgactctcgtcaccattacttttattctagaaagagaatatggttatattttgaaaggtagtttgtttttagaagcggtacagttaggtttttttagtttgttttgttttagtcaatcacctgtatcctggcttcactcagagtgtggcccatggtccagcagcatttgaatcacctggaagcttgttaaaaatgcgtaatttcaagcctagccctggaccacttaatcagaatctgcattttagcaagcctccaggtgatttgtacgtacaatcaagactgggaagctctaatttagaacactgcttctcaaacttggctgcacagtggaatcacatgggaagtttaaaaaatattaatgcctaggttatacacctccagagattctgatttaattgttctggggtatggagtcaagagttgggcatcaagagtgtttaaagctccccaggtgattctagtcacagcaaagtttgagaaccacttatttagggaaatgcattttgggggtaacaaaatattttacaggaaattatgttatttacaccttaatgtgaattatttacttagctattgctctgtcgttaacacctgtctcagatgatccagtctgaggaagtggtagctttgattgggaaagaggagaggaaagagtagtctcgggtagatggggctcaaaagcctgagagtgggtatgacaccctgaaggctttgcagggttagaagtcaatctgggactattggataaaatggtccaagatgctacagcaaattgttgtctgcattggatagatccaaagattgtgtttctacccccttacctttcctgaaaagttaaacattgtccaagattcaataacacttttagtataattttcatagttaagacatcaaaacataaataactcaggagtaaaaagatgaacattcaagaaagaaaaaatgaaaatggaagtgcatttctttccaatcattaagagttcaagttcctatttcataataatcattgggtgattgtgggactatttctctgtcatatttttattagctcttccttggcttaatacattttaattagtctcggcagatcaggatattttcaattatcttttttcttgacatttatactcatcctctttccatgcagtgagggttggtaaatagtgttcagggggtttgattctgtaatgttctgaggccaattcactgagcaacaatctgttttgatgaaacgagatcatatatttagtgcatctacacaacagggtcagaatatctgctcagttcaaactatttcatggtgaattccaatggatctatggcattattttgagaactcagcctttaaaatcttttatattttatatttatagagatagtagccagactgttattagtaacaatattaataatattattactattaaaggaactcctctaaacctcggtgaaaccaagctgaagtgaatgtaacaattcttttgtgaaatattataattttttaaagggaaaggagaaacatacaactagtgccattcattgattcctactgccagctactggaggaaagagtgaatgctttcttttaagtctttaaaagctatgcaaacataagctatttgtttagcactggaaaacaagaacaaacagattagcttgctgtttacaaagtgttatttttcatttgaatgtcaagtttttcttttacacttatagataagtacatttctgagaacgtaccataaaaatagtaaaaactgattttgtccacaaggtacagacattttgctacaaaacccacaacttttgtttgactcccatcggtaaacctacactcaaggtgttaatttcagtttacatcaaactaaagtaaaaggacccgtgaacccaatggttcctaaagcatggttgcctgaccagcagcattaccaccacctgggagtttgttagagatgtaaattattgggtttcatctcgggcttcctgaatcagacgctctaggggtggacccagcactcagtgttgtaactaacccttcaggtgatacggatgcacacttacattgaaggtcactgacttaatgaatagcaagaatctctggaatattaagaataattcttgaggagtatcagattataaatgtgtcttacgagtccctctgagcagtgtctttcttctgaatttgcagtatgaatggaacgacaatgaaatgtacctgttccgatcatctgttgcatatgctatgaggcagtactttttaaaagtaaaaaatcagatgattctttttgggtgagttgatttgctgggttctcaaattactccaactaggatttctcttactcttgagtctgaggagatactctgcctaaacttttcttcaagtgaaattgataaaaatctcctagtaacttatacttctttggtattgtggaatttgggtgactccatagagggctgatcgtgccttatttgactggtaaattgcaagagtattgttcttactaaacagctgtcactctcttctcagaatgctctcatacaccctcagcactgaaagaaaaaaaaggcagaacttcctgccttcattgacctatattctagtatgtatgttgtgtatgtgtctatgtgtgtaatatgtaagtgaaatatagagttacaaggtactaagtgctaggagaaaaatataaaagggtctggggagggctggtgtgtgtgtgtgtgtgttgggcagggggtaggtcaattttaaccaaaatggtcagaaaagacgtcactaatacttgaacaagtatatgaagcaggagagagggagagagagagcgagagagcgagagagcgagagcgagagccagcaatgcagacaatggggaaagtgtctcagctgggggaaacagcaggtgcagaccctgaggaaggaatatcctcagcctgttaaaggaacagcaaggaggccagtgtggctggagcagattagaagacaagtggagacatggccaggacgatgttagggcatagaggaaggagggagagacaaaaggtcatgtgggccttgaaggccattctaaggactttggcttttattctgagttagatgagtagtcactcaagggctatgagccacatgatctaccttattttgtgctaggatcgcttgacagctgtgagagcacagtcctgtcacatcatatggtagaccccactcattaccttcatccatgggtgcaacacgacgttttctgctgggtaggaccaggttgtgggtgtaaatccgtgtgtggtactcagccatttctgcccaccacattgtttgccacaatcctgggccaccgattattccttgttgattctcctgctgttttcagtttggaaattttctataattgaaaaagatgtgggttttatataaaggcagaacaaatagtgccaaaacttgatatcacaatttttatgtagttgatttcatgtgctttgggcagcattaatgttttttttgacaattacatcctctcattgtttgccctcccccatgtctctctatcctgtgattctctctctaatctccttttcaccagcaggggaagtcttcattctcttgattgtgtcctctgtgccacaagtgaagatgtttgttttgtttctctacagggaggaggatgtgcgagtggctaatttgaaaccaagaatctcctttaatttctttgtcactgcacctaaaaatgtgtctgatatcattcctagaactgaagttgaaaaggccatcaggtgacattttactttcatctaagggtgagggggttaccaaatacaacaacaataaccagtattttgtgtgttcttcctgtgtgccaagcattattctgagtcatttacatgtgttacctcatgctacaaactccacgtaattccagaaggatcccacagcaggggaataatcttgatttgagttccgccagatgtaggctcttacaaaagggaagaaaaactatagattacatgttatcaagcaagttaccactatgggcaactagagctcagtcctgctgggaaatgtggagagactgtgtagaatgtgcacctgaattgtcccatcagagggctgaggaagctggggtatttatttaccacctcccatcagttattggctgagaactgcttccaggtggcattaattccccagcatttcagccctgcaggcagggcagatcttgtggtctgagaatgctctctggtaaaggggagcaggtgctggttgaagaaagtctggctagaactcatggaaaatagtaagtgcagaagggacgtggacaggtattgccatcatttgctctcaggactttgatgtaggattcatttaactgcccaaatcctgaaacaaggtaggcaagtacaggattccttacatattgcttggcctctatgctaggtgagggacttgcaatgaatgtttagaagaaacagtttgtccaatttgcctcaattgtttcccacaacagacagcaggagcgaagaatggattgaccttgcagatggtttcctgtcattataaaaaggcttaatatacctactgcttctgggcagctcactaggtggccctctcgagctgcgtcagagtcctgtttaacttcggaagcagatctaagacctgctgggcagggtgtgcctagaaaatctggcttggttggagtgtatgacccaactgtgcccagagactcactcacttcctccagaagacttgtgtctgtcttgggaaaaacttctggctgagatctttatgaaaaagctcagccttcctggatactcagctgcaagctacaggggacctcagactctctgtttccaggatgactcttttcagatcttttctggctgaggttgccaggggttacctgactaattctgcgttgatctctctctctctctctggtggactctgacctcctgacctgggctatacccaagggtaggaagatgagatgccagacactttggtctttgatccagaatgttctccatatcattgtttttttttttctttaaacaatctttatccttttgcctccataaaagtaagccatttcccatcccagtgctgaagaaactggcaaaggtccctttcttatgtgcctccccagtgctacctccaaatgccaataccttttatttggaaaatactactatagagacttggtcataggacctgattcatttgtataatagaagaggtgtatccaattatccgtttccattatttgatataaagtcattgtagatgtactctgacagaagggaaattcttgccaaatatgataactttgcccttaaacacagcagtcacaaatgaataaatgccaaccatttatacatttccacacttacaactcaattttccaatggagctgttgatgaacctaatctaggttgcaaggcatgaaagatgcataattgtcaaagacttatatctttaattagacctatttactttgctcttatgattttaggtgtatattccttttttttttttttgaaacagagtcttgctctgtcacctaggctagagggcagtggcacaatctcggctcactgcaacctctgcctcccgggttcaagcgattctcctgcctcagcctcccaagtagctgggactacagacatgcgccaccatgcctggctaggtgtatattcttatgataagctctattatatcctttcaggaacaatgatataaacagtaaataactacacacaattttattttgtctaagtgtcccctttgctgttttttgcttttgcaaataggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagtaagtggcctttcctagacttaactatccaaaaatagaaagatttagagaacagtaactggaaaattttcagtaagtggtagattgtatagaccaattacttagttggacaattttgcaactaaatactctgatgcattccctagaaatttaatagatcagcagttcatcttcctagtaagcccacacagtaagtatctggcctgggatttattagcatgccctagtaaactccatcccagccacagtgtcatttccctcctacaaagaggcactgcaaactcagcaggtatgggtgggctcagggaatggttttcactgctctcactgctgaatccttttcagtctaaaagtggggactggattgctggagactggattgctgatgggtttgaaccgtgaaattgttcaagaatgggctgtgagatttctttgaaactgctactcttggttttttgtttgtttgtttgtttgtttgtttgagacagagtcttgctctgtcacccaggctggagtgcagtggtgcaaccatagttcactgcaacctcgatttcccaggctcaagtgatcgtcccacctcagcttcctgagtagctgggactacaggggtgtgccaccacacccagctaatttttgtattttttgtggagacagggttttgtcatgttgcccaagcttttcttgaactcctgagttcaagcaatccgcctgcctcgccctccagtgctgggattacaggcgtgagccaccgtgccctgcccaaactgctactctttactcagctttatttctcaccgggtagctcagactctatgtcattctcttagttcatggttaggcacgttctaatttgttcctgaactgaagatcaaagttcattgaggatttaaattaccagagacctgatttctagtcttgctttgctactagatgtttttcttctgtccttaagaaattgactcttgctgggcttatggaatgaaggttaatattttatagcacctaacaaaactctatgcacacctcagcttgtttaaacatattcttgattgaatctctatatttgctagtctattttgctcaattttcttcactatagagatttttcgtttctccaaaatagctccatctaaaatattaggcattcaatgtgtaatacattcatatggttaaaattaaaaaatacaaaggtgaacatgaagagtcttccttattaccttttgcctccaagttatcttcccaaagccaaacaaaattatcagttccttggagatacacacacacacacacacacacacacacacacacacacacagtcttttttcttttttctcagacatatatatatatatatatatatatatatatctcctttttagtatacaaatatatacttttttagtgtgtatatttgtgtgtatgtgcatatgtatatctcatttcttagtatacaaatgttagcaggctataaacaatgttctgcatattttgttttccacttaatggtttttttaaatttttatttatataggtgtgatccaccacacccagcccacttaatgtgttttagagatcattacacattagttgcacaaaaaacttctccttttttctggcagtataatttcccatttcacgaatggattgtaattttgataaacagtcatctattgatgaacttttatgttgtttccaaacatttgtgtttacttaaaaaaagtgatggcttaatataggtaatttcctgagtatgccaatatacccttaagatgaatcctagcagtgggaaagctgggtgaaagcatacgtgcaattgctaattttgataaatattgccaaattgccctcaatagcggctgtaccaatttatactcacatcaacagtttatgaaacttcctgttcctccctcccagcctccttaacacagattcccctgaaactttttgattttcatcagtccgattgattttaacataagtatattaaggcaaccttgctctatttaacaggaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactccttgtgcttacttatgtgctggggcttctttacgttttgtctgcttttcagttctatgaacaaagttatcatctgagtgtctggggctcctggccttccaaatgtctcgtgatgtattgcaaattctcagattgctttattgtacaaagtaattgtaaaactagtcccaaaaaagttgtgtacagagtgtatacaccatcaggttaggaaccaaacctgatgctgcttttatatttctcacagtcactaccccagtgtgagcacagcatttcttagatacctgaagactggttaaaactagattaaatagattaacatatctaaactctatgcattaactcattctatgtggtgaccagcataatgttacaatgactttgtacttcataaattctctatggtaattgtggatcagttagccaagtgaagggaagatgactttcataatctgtaaaataacctcaaaataagggtcatttgaggctctttgatgtcttaattatctgatacccaggtagcagagatagagttctttgttattttacgagatacaatcaacatttcgcatcaccatctgcaccatcctgccattcactgggcccacagcttggggcaacatacaatataatttactccttggctaagcaaccatttcatagacagttttttcttttatcttgcatatgtcccatcttctctaatatatcatgaaaatcttctgtcaagctactgctatctaaacatcctttgtagttgccacaagatctttatgaagttcataaacttggttcagcacagtgcaagaataatttctgcaggaaccaatttcatcatctaacaccttcaaaagagcctccccagacatggcaaaggacccatgtgcatgtgacatagtgtccaactgtaacacagaaagaaaaacctgcttatttccgtaacttaattttggggttttctccagaacagtcctcactgtgcccaccaggtaacgcttgtggtccttctcctgtgtagcagcatcctctgaaaggatggagactctacaaagagggaccggtgcctgggcctaggatggaagctggcaaacaatggcccatgggccaaatcctgcccaccacatttttttgttttgttttgttttgttttattctgttctgttttgagacggagtcttgctcggtcgcccaggctggagtgcagtggcgcgatcttggctcactgcaacctctgcaccctgggttcaagtgattatcctgcctcagcctcccgagtagctgggactacaagtgtgagccaccacacccagctaattttttgtattttttagtagagatgcagtttcaccatttggccaggctggtctcaaactcctgacctcaggtgatctgcctgactcggcctcccaaagtgctgggattacaggcctgagtcattgtgcccggccaccccaccacctgtttttgtctggcctgtgaactaagagtgatttttacatttttaagtggttgaaaaaaattagaataagatttcatgataaatgaaaatgatatgaaattcaaatttcagtgtccataataaagttttattggaacccagccacacccattcattttcatattgtctagggctgctttcatgctacagagcagagctgagtagttgaagtagagacggcatggcccacaaagccatttacagacaaactttgccaaaccctgggctatacggaagcaaggattaaatgaaactgcaaatgcaaaaggtccattgtggagctcacagaccagagaaacatagtgatcaggccagaaatggcaggtgctagcttcctcttctctggtgtctctcaatgaactattcttcctaatttgagagcctcctccctccacatttaggtaaagatggggccttaggtccaatctctcctcctcataaacccactctcaagatagaataaaaactgagaaactcaacttgctaaatctactgtgttggaatcttaataaactcaagtagatatgaggtgagttttgccagaccctcttgactttgtgtttgacttctggtaggtcagaaaatgctcagcaagacgagggagcatctgttggattttacctagcaagtgatttatctgaacttgctctgcagcctgccctttgggaaagtttcttaccacatagtcttcctttttcttcactgccaaaagagtgtaggcagacacttggtttaagataacaacaataaattctgcatatcaccactaaacacagttatacagcattaaaaaatcattgtgcacgtttgaaatactatgaatagtgttcttttacttggctcctcctagcatcactggacttgttttctgataaacctctagtggtaccttaaattgctgcctgacttgatttttgcagctaggcttctaagcagggctttggcctcttttcatagtaaacaatttttgcaacaagcgtgtattttgttataaagaggattatttttaaaaggtaaatatacaaaaataagaagaagaaaggaaaaaaatcttcgagagggagaccagaaagacccagcttgtgcatgagctcatcctctgagtggggaaacagtctttggtgcaggaaaggctggcacccccatcggactgtgggtccattgagaaagagggtttgggtcacctgttctttcttttctcttgggtaggaaagaacctagcatacagtcaacactacaaaaaaaacccacaaaaataaaatatacaaatgccctagtgattattttgagttacatttaattataaattgagatttgtgtacctctgtaaaaaatagaagactgttaacgagtgtcaattctgtgttgaaatgaaggaattattaatgaaatgtcaagaaacaaataagcaagagggagcattgaaactagctggtggctgaaggacagagtgggatgagagaggatataagtgtctctgagagattattgactgatctctggaaaatagcagcagggtttaattggggtgtctcttcattgtaatggtgccctctacagaatctgtctctcccttcttgacattatagaaaagacaatggaatactcagaaagaggttaaaacctgtgggttcggtagcaggccgtgaagaagggcatccgcaggtactgttttaaaggtgaaatactatttagtaataggcatgagaagctaaaatagtacttctgtatttttgtgttaagatagcagactgaaatagaaacttcagttatcaaactagccagaagccccagatccccaaactcaaggatttaagatgattagacagatggtcaccaaaattagtccacaagtagaagaatttacagcaccatgtcatacatagttcatcctaatttctaccaacttcactgggcaacaatcaatcgttttatttccctcaatgactcttgttatcttcagacctgaaactgattcggagaccatggggcccacaaacctaatcagagtaacatgtgttcattgagtacacatgtagacatgagaatctccactttccccttttttctcttggtaaaatgttcacaaatgttcaggtaacacctgctactccagccattcaggccttaaatctgcagctctacattttgtatccaggtcttgagatttgggaaatagaaaatttttatctaaaaaatgaaagtacttttggttatcaaactcagacattgaaatgaacgtgcagttatgtcgttctccccctttaaattatgtattcatctcttgaaactgttcactattgccacaagtagatatatattaaactaataataccacattggacactatatctcataccctaaaccataatgatatatatctaatcaataaccaatgttatttctttaaataaataaaaatttctgacaaacaactctgtatcagcccactctctgtccctttgtttttgtctttacaactcctcttgcaactgctgctaatcaaagtgtagattccaggcaacttgagtctttgctcccaggttataatccttaaacttgacccaaataaactgtctacttatataaaaaaaaaagatgattagaaagcaacaatgtaattaattctagaaattgtacctcagcatcttaaaaaatttgctttatgtgaacatatttaatgtattatttaatattatttaactttagttttatacatgacataaagagtcattgtactaatctggatattagacgagaattgtgccatttggattgggtaatggaccacctaatgataccccaccatataatgaggtcccctagggcagttcagaacaactgcttcagtaacatctgggagtttgctaaaaatacaaattcacaggctgcactcagacctcctccgccaggttttatggaggtagtttctaggaatcttttcataaactctgcatttgattatcatgcctactgaagtttgaaaacattgaaatattaagacaaatgtccacatttctgtacttggtagtatatttaattgttgtgacaatgacaatataaagaaaagaatgaatatccaataagtaatttttcagttctgctcagtctttacacatgaacacagtctctatattgcagtctgaaaatgtgcttctgctataggctggaaagtgttatatcaggagcagcataaacaaaattactaatttgatctaaatttcaagggaatcttttgtttcctgccttgttcatttctaaataatagacaatacagagaacactattaaaaactagctaaattacattatgccttgctactttccttaaaatgaatgaaaatgctggccagcatctatattcattcttatcagcactcctaatttcttcttcaatgcttgtctttttcccgaagtggttcaatggaagacaggagttgctgaaatgtgggacgcttttctggaagctaaacaaaaacaacaagaaacccaatgttttagatgatgagagttatgatcaatgataaaatatataaaatatttacatctaagtcctaaagctagcaacttttaaaatgtggaaacacaacagacatttcaattgaggtcagaaataaggcaaggacgctcactataataatcactcttaacattatgttgaaggtgttaaccaatgcaattggaaagcaagaacaattagagccccaataactggaaaagaaaaagtctctttatttgcaagcattttagtgtatacctggaaaatctgagagtatcaatgttagaacagacttaaagaataaaggaattcactaggagagcaggatataaaattaacatacaaaatcaatagccttcacatatacctacagtaaccacttagaagttatcatgtaagagaaaaccccatttataatagcaacgccaccaaaataaaacactaaaaaagtgtgcccctctaaaatggggacgactttaaaacactgctgaaaaatgcaaatgtagacttgactatataaaaaagcatccctggccaggcgcagtggctcacgcctgtaatcccagcactctgggaggctgaggtgagtggatcacttgaggccaggagttcgagaccatcctgaacaacatggcgaaaccctgtctctactaaaaatacaaaaattagccagctgtggttgtgcacaactgtagtccaagctactcgggagggtgggacatgaaaatcacttaagcctggaaggtggaggttgcagtaggcctagatcataccactgcattccagcctgagtgatagagcaagactctgtcttaaaaaaaaaaaaaaaaaaaaaaaaagcttccctcgttctagactaggacaagtcagtatcataaagatgtcaaatttcccaacttaaaatacaaactaaatacaatctcctcaaaaatatgaatgagtttgtttcaagctaaacaaattgattctttggtttatagaattaaaaaatatgtaaaaatggctaagaaattatacagtaaggtgagcaatgaggggtgctagcctacctagtattcaaacatatcataaagtctctacagaatcagagcagtgtggtgctagcacatgaacaaagagaccaaagaacagaagaggaagtctccaaatagattcaagtacatatggaaatttagtataatattagggtggcatttcaccaccattaaggtggtatctcattcctgatgataagatggtcttttaataaatagggctgccaacaacagaatagtcacataaaattatatatttggagccatatttaataccatataccataacaaacacaaactatatcagaaacataaatggaaaataatgcaatcttaaaagtactagaggaaaatcctggcaaaagtcattataatttgggcatgagaaagcctatctatgacaatttccagaagcaatgaaaacaaatattgatatattcagctatattaagaaaaatttctgcatggtcaaaaaaagaaaaaaaaacccacccaccctcaatcaaataaagctgataacttaaagtattactctccctaatttgtaaagagatctttaaaacctgacagaaaaaagaaaaaagacatgacagttcagaaagacacaaaatgacccttaaaaacgtgaaaagaggctcaacctcataatgaaagatatatatattaaagctgcactaagatgccatcaagctttcacttatcagattggcaagaattcttcaaaagcttgacaatgcactccattggtgaggttataagaaaataggtaccctcttacattgctagtgaggcaacgcctatggagaggagaagttcatatgctactttttctttgtaagagagaaagggaagcaagaaaacacacatctatacatatgcttgtttttgcatacagaaataccagaaggatgaatcaaaatctaatgaaaattgtgaatgttctttcaatctctttatgaattgagaaagtcacaaaataaaatctaaaaaaggagggaaaacccaaatctataagctctgctcattaggtgcaatgttttaactggatattgtcgtctcagtgcaaacccctcttcacagattctgccacaattcacatgcttttatgaaccaaccatgagaagaaggcagttcctcccgactcagggcccctgtccatcctatggtgcagtcatattcccaggagctagagtagggagggccagggaggttctgggcttcaactgaaaaacccttgtctgctcttcttcatctgacaggtgacagcagtaagtcagcctggctgtgaccaactctacccagacccagtctgaccctctagtgctgcctgataatcttagacacaatatttttgtttgtttgtttctttaaaaagcctttattcattttttaatcagggaaaaggacaatctcaatgtcaatttgtcacctaatcataaatttgaacttacaggattcgctaccctagacactgcacctactgacaacacacaacttaaaaatgaacttccttccattccctaaagttagatagttgcatgttgaatgaataaacagaatcccttccaacagctaacattggtacttggcctaggtgtaaggacaaatagcgaaacagcagcaaaaaagaccaagagcattgtaacttgatagcccagcaagtacaatcattgtaacaaagttgtaggcaccaaattaaaaaaaagcgggggctcatcagcataagaaacttacaccagttttgtgagagcactcgttgtgtgagatcaactactctgcctgtgaacacatatgccaatcccagtagctaacattgaggcaaaggtcccaaaatccaactaactacactgaatgcaaaatccaaagcttttatttatccaggtcctctgaaacctggatataaaacagtttcttttcaatgcaccatgagggtccactgtaatgattcagtcacatttggattgagtaacgcacctaagataccttttccactattcctgtctgaatacggaaatgtttaactactcagttcatgcactaactgtgttaaaagttagatttactatcaagactttttctaaactcacacagcattaacagttaactgtaatacagcttcctcagaatgcccaaacagaaaatattttgttcagtcttgtggtggctgtgagaaaacaaaatacaactaaatcttattttgccgctatttcaaattttaatctaacttttgaccaaactcatacctttctgccaaaatccttgagttataactactttcaatgctcttaaaccatagctcctaccagttttacagtgctccttcctgtgtgcatctgaatttttatcaagaattaaagtcatggtgttatgattacaaagctttttctaggtgaccactgattaaagccaaagtaattaacgattcaaagatggctttctagagacaggaacaaagcaaaaggagctactaacaattttattacaatccagaagaaacatggtctactagaaagtttctactaatcacaacacttatgtcttcctctcaggacaatcttcagataaccagaggggaaagaacattattcaaaagtgtggtagaaacctcagttaacagggagaagatgaaaccttaagagcatagaatacatctacaaatcggggaagagggtgaccctggctggctatttataaaggaccacagtttctccctatggaccaatactctctacaaaatcaactgtgccttaattattgcttactttaaatttttacagagctatgtagtacctgatacagaaaagcaatcacataaggaagcatttatttgaagtttaacatgaaatcatacaaataaaatgtgtataaacaaatccatattgagtcataacacagatagcaaaggacaaagtaaaaacaaagaaaactaattggcagccatatacagttggacactgtgtcttgtacactagaaagtcttttacaaaataatcatcttagatcaacagaagaccaatcttctatgtcatcctgcaagatgggttactttaacatctcatcctgttttctccaatgttctcctttagtatggctggtaattgttttggtgattgccaccccctcgagatgccttgccataagtgctctgttggccactgtagtctgcatatctctgtccatatccatagttcccatagtcaacatttggggtagaaaaaggtgtggaaaccttatatactgttccaaatgccatagggtgggaaattacagagtgctcatattccaggtgataatttaaggctaaacgaaaaatattgttatgtgatgcaatcagcaactcttccaaagtgggactctaggctccggtacaagagtttagaaactatttaagttgtgtgcgtcaatttattcttagcaccaagcgtactgcctggtgcatagatgctttgtaaaaaaaaaaaaatatatatatatatacacacacacacacaatattaataacagaaaaacagcactgtgtaccaggcattgtcctaaggcctttataatgctaattcattcaatccacccaaaaattctatgaggtaagcattacattacagtgaagcacagagataataataggaggtttgtacaaggtcatataactagtaagtgatcacgacaggattcaacccaggtattctgatgccagaacccacaggcttaaccattacaccaatttgccttttactagtgaataaatgaatgcagcattaggccctttgccattgaggaagtaccatataatgattatgaacatgggctcttagaataatgttctcaaaagcctccttttatcatgttactgctcaaaagtcttcagtggctcctcatcgcctacaaaaaccaaaactctcacttgtctccttgttattcactttgcagaacctgaaccagacctactcttccaaacttatctccagcccttccaacgatagcaccagcctagcgtgccatgctcgttcctgcctccacacctttgcttgtgctgttctcacctggacacccttttacaatgtctccactcacatacttaggaaattttgtagagatttcctgcttcagctcaggtaccaatgcattcaagaagctgctttctctcaggaagctggtttctctttcctctgagcgcttcttgtattaggttggtgcaaaagtaatcacgttttttgccattgaaagttatggcaaaaaccttgtctttctaatttgggcacataattataaactgcttggtgttttcagtcctatttgtgtgtgtgtgtgtgtgtgtgtgtgtgtgtgtgtgtgtgtgtgtgtaaagactttgagttcagtgagggcacaaatggagcctcatacttccttaacaacctccattagttctatgaacttagtggatgctttgcaaattaatcccctgaagcttcagctccatgtcttccccataagatacccttatcttctacctgcatggaatgcttttattcagtgtttgggaggcagggggagagaagatgggatagtgtccattatgtgttctctacgtgtggttagatgttttctgctacttattttctactcgaggagctccatagtgttgcagttaagaacagggacgctggcactagctagtctacttcttactagctatgtgaccttggacaagtcacttaacctttctgagtccttgtttactcttctgttagtttcagcaatgatagtacttacctcagtgtgtttcctgatgcataagtaagttaatatttatagtgttcttagaacactgctgtatgtaagttgttcagtaaattgtttggcacccgcatgttttagattacaaacattttagggcaggggctgaggaccattctctcctgctctgtaccagttgctttccctcaaaaaagctttgtcccagtggtgatgcagtaatcaccccggatgtgcctgagaccccttccagcccacccagagaatacacttgcctcgtgccagcagctgtacatgatctggtagatggtgtccgatgccaggtggggccggtaaagcctgtggccctgggagaccttcagaaccacctgggagttgtcatacaagtcatagggctgcttccccaggctgaacacctcccacatcaggatccctgcaccacaccaagaggtggcccagataaaaaaggaagagagctgttatcagaaagtacagggtttgtggagtgagtttcacacaacaggtcaggaaggctatgggtcagagtgagggatccctgggaaatgagtcccagcgaaatgagcagagcttggaaggtgagcctgtgccattgcttctggagcctggggtttcttccccatgttccctgatacctgtacatctgtgcttgctggaaagtgggtgaatggcaagggcagccacagaggaacctcttggggccaagacacaattcttgcgctagtcctgtgaagtgtgagttgtcagcctcacagtgacaactacctgtgtagtgagcccctgccccaacccttatgaatgagtggttgatgtcctcacccattccgtctctcggctttggcagctgcatgcaacaatttcattgcacaatcagattacacttcagtggaaatttcaagataaaatcattgacaacattctcctttctattgctgcagtggttcctaatctcatatgggccaggcacccttttgggaatctgatgaaagccactggccgcctttgggaaagtgaatacacagcatttttgtgttcagtttcaggatgttcacagtcccccaaaactcatccaggaaattgcagtccccaggttaagaaatctcggttctggtcatggtggtcaggaggatggcatttgggatcagttagacatggcctttgaagttgacttctgtcacttacaggtgggataagtttctcaacccctctcagcctcaggttctccgtctataaaatggggtatttgcacctcatggacttgctgtgaggattaaataggataaaacacgtaaagcacttaaaagagcactgagatgtttcaagtgctctctaaatggtggcagttgtgctgtgctggccaaggtcagaaaatttcaacagtcagccagtttcaaatatttataataaagcaaccttataaattttatggctgcttttttcttttaaatcacacagtctaaaacaacaatccaaaagaatgcagtttcctcataactttctctttggaaggctacacacttatttcaaaatatctgtggggttcttctttgggaattgctatcagaggccctgattcattttgggtatcctcactgggatatattgtttaagaatgaatttagtgtttataaacagctggaaaatatctggggccaacttctatggcatacggtaaataatcaagctgggtaatactattttttggtcaaaaacaagctgtaattattaaattagggcattgattttttgctatcacttataattgatgcatgattatcaaaactaattttttttttttaagacagcctcttgctgtgtcgccaaggctggagtgcagtggcacaatctcagctcactgtaaactctgcctcccaggtttaagcgattctcgtgcctcagcctctcgagtagctggggttacaggcgcgcaccaccatgcctggctaatttttgtgtttttagtagagacgagtttttgccatgttggctgggctggtcttgaactcctggcctcaagtgatctgcccgcctcagcctctcgaagtgctgggattacaggcatgagccaccgcactcagcctcaaaactaattttgacaacaaggtacaaaagttaatcagtacaacatggcatcaagtattcaccaaaatgctatctttgtgattttttttctttttctgtgtgtatttgattatctttttttttaacaacgaacaatgccacttttacaaagggaatagttagttttaaaaattgtgtcacatagagaaataaataactaaattttccccttttcaaaggcagtcttgtaggtgacaagcagtaaatcaaattacacttatgaactaaggcatgaaataggtctcagtcacatttggattacttatcctgggtccatccgacagtgattgaaaatagccttgatattctcacaacatagaaaaaatatcataaaatgtagtcacaatttaagaattgtttcccttttctaatgacctttactaaattttaatatagatattttagtaaaataagagtcagcaaacttatcctataaagggccacatagcattttagactttttgggacatgcagtatctgtggcaactactctactctgctgttgtagcacaaaatgagccatagataatacataaatgaatgggcatgactgtgttccaataaaactttatttacaaaaaccaactgacctggtcctgggctgtggtttgccaatcccaatctaaaatataaattttaacagagaggagactacttgaaactaggcacgagtcaaagaaagtaccaagtcatgtgtggtgtttacactaagaattggtcagcgcagaagggaatggctatcccattattttcatgcagaaaattgatgtcttttcaacgtttggggctcagcaaaaactcagattcagctttcaatgactccttttaaaaaattatttgaaatcttcactggaaaaactttcatcccctttgatgtttctaaagacagggtcttaactgtgttgccccagctggacttgaactcctgggctcaaacaatccttccacctcaacctctgagtagatttatcccttttgattttatgagaaagtgctagcaggatgtaaattacaagatgtgtagaagacatcctaataccaaacccaagaaaataaatcaccttcttcaacagcttgcttctctcaggctatatgtgtaacttcgtaagtgttcaagggcccttttaaaatggacattgctactcttgtggtttcctgttgtggaagtaagcttatgaaacagggccaggtcgtatgtggccacatccttaccaaatgcccatacgtctgacttgctgctgtatttgaagtaatgaaacacctctggagctgaccacttgactggaaactttgttccgactgaactgacatactggtcatcaagaacatacctgccagggaaaacacatcaacacagaccttaagccgtggtggtacaggctgatgaaggacacggaagccacccagagtctcagtcgattgcctcagttttctggaacttttgtttcctttcaatgtgtgggcttagctggaggctgcttatggtttgtaagcatttaaaagataatatctaactgaatacagcaaaacctcaatgcaaaatgtttggtaaatatagaaaaacacaaaaaagtaaagataaaattactcagaaacccaccctaaagataacctgtaattctatcactcaaaatacatttttctttgtactttctgatcttatttcccacatatataagttgaacaaaatagggtttctgttatattgagatttgcttttatttttcctttaatggtatatcatgagcatttccccatacggttacatttctactgcagcatcatgtttagtggctgaataccattccctcttattatttcttcttatggaaggaattaaacataatttatttaaccaggtaaattgctactaatcttctgctgttatgaataatgatgaacaaaaatatcctatggagatatcaagaccaaaatacatgttgaagaatgttcactaaactctttgtttgtaataatgataaaatgaaagcaacctcaatgttcccaatgttgaggagtaattaaatatattatggcacactcacacaatagaatagcacatagccattcaaaaatgagataactcttcaccaaaataaaaaggtgctcatggtatatggttatgtgcaaaagaaagtgtgtataatgtgattccattttttgtgtgtgggaacaagtagtaaatgtgcatatttgtgtatatgattggatacacttaagaatgaggtctgaaaggatacccaccaaacatctgagcagtggttactcctagcactgaaaatgagggacagattctgtcagaataggtagctaggcagacatgagcagggcaggagagatcccccacccccaacccaggaatattaggtgaccgtcaggtaatagtcaggcagttgttaaccgtctctctaaaataataattggtcgcagctggtgccagggaacggcagtctcccaatagatagaaaatacttgaagctgatgatcaacagcttcctgatatgatctcaggagttgggcaactggggtcaaacatgcgcaataagaggcaaaataatggagtttaactggtatatgaccttcctctaagaatactcgactggtaaaagaaaaatgcctcaaatgagcatgcgcacaacctaagtaaacacactgtgcgtgcggcccctcccaagtgctggcaggctactgctcatgcagacagcccaccccaagggaagaatcaggggagaaaagatgcaaactttggaattatgccaatgtatgaaaccctaagtcaagggccaagtagggcagttggatctctcaagttgccctcttggccctcttccaagtgtgctttacttcctttccttcctgctctaaaactttttaataaactctcactcctgctcaaaaacttgcctcagtctctctctctgccttatgcccctcagatgaatcctttcctttgaggaggcaagaattgagttgctgcagacctttacggattcactgctgctaacaattctactttttactttctacactaatgctatttttcttataataagtacatattatttatagtattaataaaagagaacatcatatatttgcccacaaaaaccacaacaaaaacaaacacataatgactcttaattagagaaaatagaacaacaggagttatggcaattggaattttggatataatttacacaccaaatgttttagagatctcactgtaatttaaaatagctggctgggaagatcatccttctaaatattaacaataatattaataaactacactttcataggattacatacagtacattccatacttttctccccagaacccctcagctccccagacactaccagatacccagacactaccctggcactcctggtttggggatgctcatggcttaaaccttccttcaagctagtaggagcaaatgaagtcacaggcatctaattgatgtggccacagagcttcataaccaacagggctggtacatggcttccaggatctcttactcagagcaagatctcttttttctttggatctttttttggaaaagtctcccttttccaaagctacagagaacgtgaaagcatgttccctggatgctggcataacagccaatattcttgcttttagagtaaaggtggggatcagttcccttgacctgggagcttctccctgctttaagaacctgtcagcagattttttttttcttgctcttgagaaagctacatgtttggagggtcagtgtaggggactcagtgtatggtggttggagtgacggtagctagtacacagctaaaaggacatcttgccaatatttagggctcccagaggaattaccatagcttccacggtatgtgccacaagggtcaaaaagcatcccctaacactgaattttggagaagacattaaaaaaacagatcctcctatggcattttagggcaatgtttgctaaatattaacctggccttggtcaactcttcagatctcttttaaactgcattattcaccttcagaattacatattgagtcagcacaactttatagttatattttgtctcaatgtcagggatatctacttgccctctagactaaactgtggtcattttgagagcagggactatcctattcatatcagtatctcctgcaggactgaggtattcagtaggcactcaataaatgttgaatagaaaggttaatgtgtatgtcttcgttttctttgcaaaactatgaaaatttgctctgaggcttaaatagtattaggatacaggattaaggtaaaagaagactttaaaaaaacgatcgtaatcaaaataattgttccacatcattaagtggtggtagaatgcccatcaagagaatctcagagtctagttttatgccttactttgtgatgcagaaaatctaatgatgtgacaacaaaaattttcctagtgtgaatgctctacccatttgaacatcaggttttttttttaatgacagacaaaaagagctagcagatccttgattgagttaaaaaattataaatatttatccattctttttaaatggctgagtagtactccaaaatgcttgaggggatggataccccattctccatgatgcgattatttcacattgcatgcctgtatcaaaacatcacatgtaccccataaatatgtatacctactatgtacccacaaaaattaaaaataaaaaaatttaaaagaaagtaaaacaatccaataatgtggctgtaacatgttgtatttaaatttaggatctttcatggaatttatttagaagtttagaaagtatcagattgttgaaagaactaaaagtgacgtcagataatttctgaagaacaatcccatgggagggacttaggtttctaagcccccaattcagagcaccttctatatccccacaaagccttcaattatgatggtgttcaaatgtgaatggaaatccccctttcactggttgccctttcggaattggcttaccttgtcattccaaagtcagatactttcacacagagatctctgtccaccaagcagttacgagcagcctttaaaggaaaaacaaaatcaccaagtagtgaaagtgaccaagaatctcctgacattttcctccttttgagtagaattctcagcaaaatctaagattacctagcacaacccccaagtgctctaaagttgtcttttttttcctttgcaattttcaatttttcaaaataaaaatgttctcccatgagtactttcaaccaagcatccatccttgtcaaacaacatgaaaaggtattacactttaaacacagaaccttagcttatcaaggaaagatgagaaatttctagaatctccctttccaccaccagataccgtgcattcacagatgctttgaaggcataaacgctgcacctgtgacttagggaacacactggcctagtgtgaatgcaggtctactgttgagcgcaagaaaatctctaaagataaatcccagtgaataccaaacacagcattccccactctcattctgatccaaagacacacaaattctatttccttttttttttttcatgttcagttattgtatcaaatggctgctttagcactaagcccttcctgaaatgaaggtcaatttgggtttaatggttttgagagacactaaattggcagaagtctttcctttccatctgttctccttcttgccacaacacagctatgtagaccttaacagcttattttattctcactgttctttccatacttcaaatatttttacattgatttttgtaaaaatgtcatttgcttaaacaagtcacaagtgagcaaagtagaagtgatggtgcccttcttgccttccctaccagtgaccccatctccagaagtgggagccgtggactgctttcttttctttgaaattccaatggctttgcttaccaagtcccggtgtatgaattggtgactctccaagaaggccatgccttcacagacatcgtagcacatttctaagagctgggaaggttcaagtccttttccgtgactcctcaggtaattcagcaagcagccattgcttatatattcagtcactatgtatatggggtattcctttgaacacactccatagaatttaaccagcttgggatggctgagtttcctggaaataacagattttcaaagttcaatgctccacactccattttctacatgtactgaaaaagagttgctctcagatttctgtttgttttaattcccacccccaccctctttcacttgaacagagaaagagagggaaaagtttaggatcttatatttcaagcacaattatctttggaggcaaaaaaaatttctgcctcatatcagacattggtgacttcagcagcaatttagcagtttgtgaatgctgtattcaactttcctacagagaatcagggtagcaatggtctgttgcactggaaaccctgttgagtctcatggcatcctgggaacgtccatgacttatttagaggaagccacaccctaagaacaatcatttctcttttattaactttcacatctaaatatttcattctaaaaaagtatattagaatacgaagaatgtaggctaaatgaagtcagatgaggtttgattttttttcttttttaaaaaattattttacattattgttttcattatttttgaataacaaatttataaaatactaaccatatgatgtagcacttctggtaggtcatgatggcatggacactcagatttttttttaatgagataattttcattttattatggcagatggttttaccgtacctcttcccagcaccctttttaaaatgataaagttaagtagaagaaaatggagcgtaatgtagctatgggctattatttagtaatcagatgttccagatggaagtgtttaatcttgcgaattgaagagtcctttagtgtttcttattaatttaagtaatatcaatcttaaattttgtactagcctagagcttaaaagaaatcaagcaaaccttcttgtaaagtgtgattcttctaccttaattacctttctcatgatatcttttctcacctatctttttttttttttttccatttgtgagatgcataggttaagctaaatgtctagcggagtctctagcagtgtgctgccatatcacctttacttctccagacaggctcccgaactgctgctttactctctttactgtcttcacggttaagcataaagccttttgaaagtacttagcaactccaattactctttcttttctgttctacattgtatttcagtgaatttggacacatttttggattgacatgtactttactcttagattctgagctgactctgagtgatccattgtacaaacaaacagtgcagtagtgtagttgttaatgagtttggaggtattagcctttttggggagattagtcttttgtccataatatgaattacaaataattttgctcagtttatcatttgtcttttagtttgcatagggtttttattttgttttgttgccacgcagaagttgatttttaaattttatttttttttttattatactttgagttctgggatacatgtgcagaacgtgcaggtttgttacatatatatacatgtgccatggtggtttgctgcacccgtcatcttcattaggtatttctcctaatgctatctttcccctatcccccaccccccaacaagccccagtgtgtgatgttcccctccctgtgtccatgtgttctcattgttcaacttccacttatgagtgagaacatgcggtgttcggttttctgttcctgtgttagtttgctgagaatgatggtttccagcttcatccatgtccttgcaaaggacatgaactcattcttttttatggctgcatagtattccatggtgtatatgtgccacattttctttatccagtctaacattgatgggcatttgagttggttccaagtctttgctattgtgaatagtgctgcaatacacatatgtgtgcatgtgtctttatagtagaatgatttataatcctttgaatatatacccagtactgggattgctgggtcaaatgatatttctggttctagatccttgaggaatcaccacactgtcttccacagtggttgaaataatttacactcccaccaacagtgtaaaagcgttcctatttctctccacatcctctccagcatctgttgtttcctggctttttaatgattgccattctaaatggcgtgaaatggtatctcactgtcgttttgatttgcatttctctaatgacaagtgatgatgagctttttttcatatgtttgttgtccgcataaatgtcttcttttgagaagggacactcagtttttacaagctcattaacttgttcttatatgattaatctaattttaacaatttacaggattattctgtaatgttcaaatgtgtaaagaaatctaaatttaacatgtgaagttttaaatattttaataagaatttattaaatttttagttattttcttccttcttagtttaactatagggacaatgcttatcatgtcactcaagttgatgagcttgggagtattcatcttttagcagttttggagacctgcatggtactcaacactgctcccttcagagggaaggcttgtgatggcacaaaggtccatcacccaataaagaaatattctttgaacaaattcagtcactgcccaggtggctcaaggtgagggcgccatattaactgatattccccacatgataggcttcagcttgacccctacatttgggattagactttttcccattgtggcacagtttgacgtttttctttatgattaggtgatgtttttaaatgacttcttgtgatttttttatggataccagcttggtaatggcaaaaatatgttttaaagttacaccaagacacagaggttacatcatgagcctgtaaccatgagaaaaatctggcagaaagtttctgccagattttcctgtcctacaagaggagaatatcaggggcccccttcccagttttatgacagtccaatcactgaaggcagaaccagttacataattgcaggcaccaatgcaaaatgaaaatgcaggaatccttgtttaaaaatcacgaagtattacttcaagatgacaacagcagagcattaaaccaagcacagggcccttctaggtactgtgcccttctaggtaccttctaggtactaggtacttctaggcccttgcaactgtgcagatctcatatgcccatgaagccagctagggccaaaggcctatattcacatagcatctctgcttccaaataattttgcaattatggtaaaagtgattgttctgactttcctaattcctttggtcttcataaaagaataactaaagaaatggaccctagaatgtctgcctgggtttgtatctctgcttttcgacttttactagcttgccgactctggacaagttattcaacatttctgtgcttcggtttcctgtaaatctaagataacgatggtacaaccctcataggtttcttatggggattaagtgagttaatatttgtaaagtgcttagagaaatgcctgtaaacactgtacagtataactgtttaagtaaaaatttaaaaagtgctctcaatctctagcaggctaggaatttttttatttcaactatttttgaccagagacctactataacatcatttttcataccatactgattgattctctaaaagtagagtgaccaactgtcctggtttgcctggggctgggaggtttcctaggacacaggactttcagtgctaaaacctcccagacctcagcaaacctgaatggtggtcactctacaaagtctgatttactatgttgacaagaagagggagaattatatacaactgagagagggagagagagggagagagagagagaaggagagagaggaagagggagagagagggagagagaaagagagagagggggaagagagagagagagagaagacacagagggaaagagaacacatatgtattgccaataattgaaggctgttgtgctgggagctgagacacattaggaagagtattagactgtattgaaggtaccaagaaggaaaaggatattgttggtagagcattaattttcagtactgcattagttctgggtggtagccccactctgttcttcatcccaccatgcacctccccacagggcaaggaggccacagggccaaggagacatgcctactccgaccctatatgtccctttctctgccacccttagagcaccaggactctgaaatcacccaaccatatagtcctgaggcctctttcaggacctgtgctgactcttccttaaggagtctgtccgcttaagggtaaagcacactacccctagccaagatcattagccactagataattgtttgcgggagacaaagagggagcttgtgtgtactgaatgctgtgtctgtacataactactgaggccccttatagtgcaggacggagttgggtatgaggggagaagcaggaggggtggagtttggaagaccacaggccagaggctggtttctttgtagagtcttgggaattctcaatttgaatatggcctttcaggttgttatgaaggtatatttgtcaaagtaggagcataagactgatttgatttaatagtctgttagttcaatttataacttctaaatatttatgtctatgatacagtatgtggacttttgctctaggccttgcaaagttaacagcaggcctattagtgctaatgccctcccatgatgagggctaaacagccattccttgggaaaaacttacatcatagtctgggcctcctgaaagaattcatcttctgacatggagccctccttgatcatcttaacagcaacatcatactgccccttccacttgcccagctggaccactccaaactggccacttcccagctccttcaacaaggtaatctcttctcttttcagttcccagattcctaacaaggcaagacggacattctttaggacgagcatagaaagctttggggctaacagcttcccaagtgatttcttctccaatgggagtttcacagtgacaatataaacaagactttttattttaagttgataagttaaaaaaaaaaacccttagtgatgtggtcttgtggctacgtgataatggcaccagggtaacactattcttgaagtagctacagttatttaacaagaaggctcagagtaaaaacaatttgccaatcagaggcatcttggagaggcttagcatcaaaatgtactcgatgtccttagattggtaaaacattgtcagatttttttaataaaaaaattaaactgtgatcgcagtggatgtgatcgtcctaacacacaaggttgaatactggctctgctcagtgggcacaaaatccaggaaaaagataggacagacaatggtgtgctggaggctaataatggggtctgataagaaaacaaattgatgatcttcttggtgattctagaagcttacagatttaggttgagaggacagattttttttttttttaaagaaaatccttcctgatacaatctgaacagaggcctccttcctgtctttcctcctttttttttttttttgccagtaaattaaccatatatttaaataaaaaatgatggaaaatggagtttaagaacccaagtatgtatccataccatttcccagggacacagagtcggggaccttgttggcctttgttgacacagggtggcggagccgtgtgatcatgcctgcaatggaatcagcatcatatcatcacatcattatatcatatcccatcacattatgtaacattggttcacaactagtctctctccaaagtggctgaaaaagcaaagagttcagaatgttgtaatgcagagagagaatccattcagttgtagttgtgcttaacttgactaattagcttaatgcaaagcgaaaattaaatgaataaaacactgtatgtgatttttaaaaatcccatagaaattaattgcatgatgtaatcttttaaaattctcccccttcttttttgctatctctttacaatcttatgcttaaaatatgtatacacacagcacatacatttgacaaaaaattcaaacaatacagaagggtaaaaataataagccatctgaatttttaaaaactgaacctattactaagtttcattaagtggtcaagccattttaattgtttatatttagttatggtaaaactgggttaaagtaattataaaatttaatagtggcttgaaagaacccattaaaatatatgtatatatgttttttgctttaaatgtggaatgctctccaagtcaagggacctatctatcacacatgtacttttcccctccttcctttttctatatatttgtctggtttttatatgagtggtctccaaacttttaaaatcctttacctttaatcaactaaattaattaattaaaatgtaaaaattagtacatttctataaaatatatatttggtgtgaattataaaaataaactcacaaacagaaaatgtaagagaatgagataaaaatccaaatataaataaacatcttaatatttccttctgatatcctgtgaagtttcctgtgcatctctttgtgcaataccttttacaaaaggtacttctgaggtactacaggttaaaataaacaattgaggaaattggggcaaaggaaaaacatcagtaagagccaaaatgaaagccagaagtgaggtcaattcacctacatgcatgcaatcaagtcatataaagtaacacaaattaagtgctgagcttatggcttatttttactcactgttggaaattcatccagccactgctcttaagtggcactattctagaaaacagagggtatttctagatgtatttccaccagttgattaggcaaaaatttagaagactaatagcacccagtgttggtgaggatgaaagaaaatgttgttagtaggattgtacatttctgaaaagcagttggcaatctgtacattttaagtattcttagcctttgtctcagaaattccatttctagggatttggctatagaacccctcatatagatggacagagctatgcatgcagaattgtgcactgaaacatccattataatgtgaaaatactggacatgatccaactgctaatcaacagagggctaggtgaataaattatggggtaactatgatagaattctacacagctgttaaatagaccaaagtaagctctttgtgtttgcgtggaaagacttctaagacatgttgagtaaagaaagcaagctgagcaatatgtgtagaatgatctcatctctgtaaaaatgtaatgtatatacataatatataatttctatttatatatgtaatgattagtaagtgctagtggcagcatgcaaaaaaccctgtcataatagacaactggtccatgatccaacgatatgtatatatttctataatattttaaatttataatcctcttgcttctatacacagaaaaaccctacgaatatttttataaagagaatttctctttctaagtcacccagtttactcaatttcattctttctggcattaaggagagaatcatgttatattcctgggaatgtaagctaaaaattatgataggcaaaagacttgagaggattttttcctactttcatggttccctatataattaagaatattaggaggatcagtagtatccaggaaaattgctgttttacaacaagggcccataaaagaaaaactaaaataagttacctgctgaattgtgttgatgataatgaataagctttggaatggaatcaaaacagtagttttctgccaggtataatttgttctcagcatttgtatgcacgtggtaatgtttgacagttccttttttatcactagtaacagaaagaatgttagaacatttaaaataaacaataaagcttgattaagcaagggttaaataattattttaggtgcatttgcaaattttggagagggaagtcagggaggttttctcaggcttggcatctggtgtttttctcagtcttgaaatctggtgtaactataaaagtaattatggaagagagttcttctctgaactcaatgtatcttttggtgcacatatagatttcatgtacagtaaaattccaactttccaggcttctcctcccttagtaagtgtataaaactggaatctttctttgatgatcgagagaggcttgtagttcttcaggatcattattctgaacataaattatcttataggataataatggaggtggaaatgagagaggtaaaggtgattaggctgttgggtatcctcaccccaaaacatgggaggaggctcttttggggcatgcagaatgaagtaggtgaggatgtcctttaattcctcatgtgaacttgtaccttctccacataaaacacttttttcttttcctctgcttttatacaataatgctgaattgatgcacgttgatttaagctaatcaggaataatacacctgtcttagattgtagacaatgacatagtggtcaagaaccaggctttgagtttagttctatcacttgctagctacctatatggttatgcctggcatataagaaacagttaataaattgttgctgtatagcaaactaacacaggaacagaaaaccagataccacatatttttacttgtaagtgggagataaatgatgacaacacatggacacatagaggaaaatagtagacactgaggcttacttgagggtggagggtgggaggagggagaagagcagaaaaaataactattgaacactaggcttagtagttgggtgatgaaataatctctacaagaaagccacatgacaagagtttacctatataacaaacctgtcacatgtaccaatcaacttaaaataaaagctaaaaaattaaaaaattgttgctgtaacatcaaccgagtaaaaattgcaagtacaaaaggagaaatggttcttctctttgctgcaaatgtttaatcagcttttggtgtcagcatacaattcactcaaggccattgatgaggtttcataaaagagggcttctgtattcatttgattctttatctatttgctgatttatctacctggctaccttaaaaaatgaatctgtagtcaagaaactcaactacaaggacatctgataccaggtttggctgaaactgcagggccagaaggccacaaaacttttgatagtcaacaaagtcttctcctgattcttactgggttggctttagccagcatccaggtgttaccatgtgcaaaataatatatgagatacactggggacatggattctggcattggaaagtgctgtgggacaattcaggtacatcctattatctcaagtgtaggtaggcactctgtaaaaaatttcaaaataaaataacgtttttattacttaattattataaatatacatatatgcactgtggggtagggcacatggggaaggcaccaaggcagacgatcattcaatctagctgctaaaaaagagttgcacacatagaaatttaaaagaagatccgaatcaatatttgaccagattctcattccaaaacactgggggttcagtgtaaacacagaggtgtagataaagaaacatgacttggctattggaagcccagcttcaaattagtcacttagtagctccaggtaggtctttataagctctttttgaacctcaatcctctcatctgcaatatggtataaataaaaacagctttcccatttgctttggaggacgattagaaggaaaaaaaaagagctaactaccaataagaaagtggcttgtaaaactctggagaaggattttgatgatgatttgcatgagatgactgtggggtgtcttttatgcccaagtgttagggtacagggagtggagcaggtagaactacccttccccagcaacaacaacaacaataatagtaagtactattagtttattaagtgcttactatgtgtcaggcactgttctggatataatatattaattaactcaccccactcatagaacgtaacaatcattatgttcccctttcaaatgaagaagttgaggcacagaggctgagaaacttgctcaatgtcacaaagccctattgaaatagtagagccaggatttggatctagaagtttggcttcaaacaagaacttttagtcacacaaagaggacagagccctggaaggaagctgtgtagagaacacagcagcactgagtcaaccttctgctaactctgcacccagtggaagggcttactaagaactggacactggagtaggcatctttgctcagcaggtggctagggctgagagtatagacgctccattgctattttttttctttccaggcttagacctgaatgaaagggaatcacccgagagtctcttaataaacatttactgaggcatgctgtgctctgggcactctgttactgcctggagtggggcaggagaggattagccagagaatggcctcttgaaagtagtggggcaaggatggtggggcagtggggtgagagtggtggaggcaactacaaatggtcacattcaatagaaatactttgatttaaaattccagagtttaaagatgatgtcttatcccaaaatgtgattgtgaccagcatattcaaagaatatacgcagaaagagaaaagaaattaatccgtatcatacttacttcacagccttactaaataaggacactgtgtacattcccacttggctcgaatttctaaccataaatgctccttcttttccctgaaaaggaaggaaaatatagaaggaatagaaaatattttcatcattcttgaaaatgattttgcatggtaggtagttggaaaacaaatcttttagattgactttatatatcacaaaaggcacaggtttctcactcaatctctttatagatctgctctatgtgtatgtgtatgtgtatgaactcttcctctccaattaatatttaaccctatcacttacttttctcggtgttgtgcttatgcaagcctaatgttttcatctttaagaaatacaatttagaaccgtgtatcaccgtatcatgcctttggaaggttttagaaaaccaaagatcaagaataaatcagttgataaagttctatactcaagttgtctttctcaatgatttgtgctaattctaagcagatcaaagttcagaaagagcagattataaactatgaatgtgacaaaacgccaaatgaagtcattacattctcactagcacccctcaaagaaataagtgtaatgtttagtgtttttatcaggttaaactatatcaaagtgtcatttttagaaatcaaaagagtttgctactggcattccatatcaattagtaataaagtcagataaacatgttctgtatcacatctgctatactctggtctaggtgctaacgagcagtgacagagctgaggttaatggctacattgacggcctccgataaatcatctctcttattcatgctgtgagcagtcccctcccatactgactctgggctgagccatgtgacttgctatgaccaataggaccttagcaaacacaacaaaagcagagactcgaaaagtgcttgcactgtggggctcgtcctcttcaactactgccttgagatcaccatgctgtgaggaggcccaagtgaggcacatggagagactgcttggggaagaactgtggaactccagttgactgcctcagctaaacccaagcccccagtcaatccacgacccaaatgcagccacaggagtgcccctaagcaaaacttgcctaagaactgctggcccagctgactcacagaatcatgagatatcataaatcccactgtttcaagccacccaatttaggagtggtttattatgcagcaatacataatggacacagaagtagtggctagacatgaagagtcgggcaacccacagactggacaagcaacactacaatgacttgactatataattagcaggaaggtttctatgagaaatgcatcaacctctggaccccaaaatgcaaagagattctgtgaggctgttttaaagacaagactatttaccttttgtctgagtaactgttcagattgtgatctggagatgttaccagcaaaccagctgcaaaaggaaaaaaattatatgtgagaatcaataagttttagattgtatagcttgacactcaactttctaatctgtaaaatgaggcattggcttacctcaggggtttccaagcctaactgctccagagaatgatttgaggagcttttcagaaatacaaatgcttaagtccccactactaaattctgatgttggggtggagtctgcacatctaatttttaaatgggctggcatttaggaatctttggttttcttctaggtgacttctaagttttcctacattttaccattttgagtaagggctagagattagacctatgaagaatattccaagaggaaagtcagggctgccaaggatgaaagctcttgtcttccaatcatataatttccttgtaataatctgattataacatctattttcacatcaaatgcttttactttatatgtggagctcaacaggccaagagctgtttctcatgtgagggcaataatgtctgtggcctttgggctttagacagaatgtcctacttaggaaatgagtaaaagcagggcatgaggtgggatgtagcatgggtttgggtgagtgggaggacccactggctagaagataacatctgcagaggaaataagtacaagaggggggtgagggtagaagggaggagttataggagaaattcaatgtagaaggtatctcagaacttggcaggaatggatgggaagtgattctttgtataatctctgtatgagttttgggaagggatgtcagctggtggacagctcaataaaatagaaagagccttaggtcacccagcttggaggtctgggttctaggcataacttccctatgtgtaattgtatgactttgaacaagccacttggccttgtttgaccttggacttccctacctgtaaaactagggagagggttgatcttgataagtttgaaagtctcttttaactctaccattatggatactgtataactaacacttgaaattttaactgatcgtttttcttattagtcaacattttaaggtaatactagctaatcaaaaggcccttcaggggctgtgatacaggatatatacctacaggggaatagaaggaagaaaatcaagaatcaggagagaggcctggagaggcaaggtgatataacctatgtaccctatacattatacctcccacacacatacaaacgttgcctgtgtcataattttgctgaggcctgcctcttattgttttaaataatatttcagtattcaatgacagagcagcagagtcctaggcctaaggtttttttttgtttgttttgttttttgacagggtctcactctgtcacataggctggagtgcagtggtatgatctcggctcactacagcctcgacctcccgggctcaagtgatcctcccacctcagcctcccaagtagttgggactacaggaatgggcaaccacacctggctaatttttgtatttttgtagagatgaggttttgtcatgttgccccggctggtctcaaacttctgagctcaagagatctgcctagctcagcctctgaaagtgctgggattataggcatgtgccactgtacctggccaggcctagtcttaacacacgattttctatctaaatctcagggcttagtgcttctgtctttaatggaattgactggaattgctcatgaagttatggccataaaacaaaaactcccatgacactgatagaataggaagctgggagcattaagcacaatgtgaggtgactgagtcccagatcctatagccctataatcccgagctccttttggtatctttttgattttgaagaggacaagtcttccctgataatttcctcaatgtctgtctctcacacaccacttgcattcagaaaagatgggaatagagactgtttaaatggtcatatctgggcttacccaatcagggaaagacttctaatttatttaaatgctaaagagtacaatccacaattagaggttcactcccattggtttaagtctttgtgtttcattaaagtgcaattatctttggaaatgaccatgcgttggtcattctcagtcttttatgtcttagtatgaattattccatcaatgctttcctgagaatagatgttagggaagaaagtaaagccgaaagagagctagaaggtgataaagagaccatgccacaaagatgtggagggtacagcacatcttccccaatgcaacaggtatggctaggaccaggctgggttagagatgagaggaggagtgaaaacagtagcatgtactttgtaaaagctctcgtttcacatgtaatcaataaatcaataatacaacacttgtacaaaaacagaactggaaatatgcataatcctagtgttcaagtgaattctgtgccatttggtatcaagtgtctgagaaatgcaatatgctctattattcccacctcttcttaacactaatctttacatctaggagacaattatttgtccctatttgtagaatgatagacccttagattttggaaagaaccttaatagccacttaggccaaccttctcagtatagttggggtgaccacacgttcttatttgcccagaagtccagtttatgtcaaatgtccaggtgtaatttttattttaatttttgtgagtacatagcaggtatatatatatgtgtgtgtgtgtgtgtgtatatatatatatatatatatatatatatatatatatttgtgtgtgtgtgtgtgtgtgtttgtgttgttttttttttttttttttttttttgagacagagtcttgctctgtcgcccaggctggagtgcagtggcgccatctcggctcaccatagcaggtatatatatttatggggtacatgaaatattttgatacagacatgcaatgcataataatcacatcatggattatggggtatccatcccttcaagcatttatcctttgagttacaaacaattaattatactctttaagttattttaaaatgtataattaagttattattaacgggtataatttttaatagtgcactttttctctttctaaagtatcccaattttgatgttaaatgaaaaggttggctgggtgaggtggctcacgcctgtaatcccagcactttgggaggccgaggcgggtggatcacctgaggtcaggagttcaagaccagcctggcaaacatggcgaaaccctgtctctactaaaaatacaaaaattagctgggcatggtggcaggcgcctgtaatcttagctactcgggaggctgaggtaggagaattgcttgaacctaggaggtggaggttgcaatgagccgagatcatgccactgcactccagcctgggcaacaagagcaaaactctgtctcaaaaaaaaaaaaaaaaaatgacaaggtcatcctaagaatagagatctgtcgacataaatattgccatgttcaaggtctcatttgtgctaccctgtaaaattactgcatcattaataacttctccaaaaatattactcaatgttaataagagctttcaaacatcaccttaattagaggtctccaggaaagaaataatgagcagaaacattatatgctacatctacaaaggtaaagtgaaatgaaggtgacgctggacatacgataaatccaataagtatttgcatccaatttagataatccagttttggggaaaatattttcgttggtaacaacccagctcattgtcacctgtcatctagataaatgacccagaacattacgtgagtttacccatgtaaatacagcctcctgagacggtctcagccatctgttttaatgtctttgaggaagagagtagaagtatctatgtctaattttagaagaacccagaagactttcaatactcactcataatcatccaggttttcctcttcttcagatgaacttgactcagggaattcccttaagaagagttgagaaaaacgtgagtgcagaaaagcaccaagaaataatccaagtgcgtagccatgcaatatcaaatcccaccctgattttatccacctaaataacatttagattgttaggtttctccttctgcacctggcaaagtatcaacacagcaacagtaaagagagagtcactcaaaagttgggcttatgccaagttttaatgaaagaatggttctttgatacaggagatccccaggaggcccccagaagaacttatatttctttgactttttccaaagagtgggctcacgaaatagatgaagactccttcagagatggctgtagtatcaagttaaaaaaaaattgggttgtgtatatagctattcttatggaagagttattcaactctaggttgcttttcttcctgctagagatgcctgggtcaggtatactaatgttggtgctactactggtgtatgtcatggatgctggctaggaatagtcagttcaagaaaaactagaacccaaaagccatccattcagggttcagagaggcaaactacatagacttttttggtttatatttaccttaagtagaaagcctagctgcaaaattattgcaagtttggttagttttcctgaaaatggaagcatattctggatgtaggagatatctgaatttgatttaccatgaaatctttgaggtggtgtgattcacatttctttctttttggttactgcttgcaacatcttcactgctgctgctactgcaaaaaagagacattttataagtttataaacatatgtctacttataaattttaaatgacaaaagtaattttgttcagatatgttttagctttatttgacattttgacccacacatttcacttgtttgggaaatttaagctaatgagttactttcacattttggttctttcaggcattccaataaaagcagtagtctggcaacacttctaaacaaaagaaaattgttaataatggctcataattatcttaaacaatggaaaatctagattaataaatcatcttgactggatttgccctgattttcttatatatatgcaagcattctgtcacttatatattttaaaataacttattttcctcttagctattgtgactacaattaaaattcttgaaaatgctgatctttttactccttctggacacagatggataaggaggattttcatcatcctctgccatagatcattttatctcttttatttggggtattaagcagagcatgaagaagtgcctacctctcaaagtctaaaggctgttttatctggaatgtggaacctatttcctgtttgccttcatagtccagacttttttcaaaaccacagttcatagaaacaagtttccttagcaaggaaatgctcccctggctctatttcaaggaatgcctagataaagcccttaaactctaaaatgcctctctctccaccttcccaaagagccttgcccactagaatagcttttatttagccttgttgtttctaaaaacacttagagaatgtccatcaatagtgcatcacttaggtttacctagtatctatacaactggtacttacagacattaaaaataatcaggcaatttgcacaacaatgtaaatgtacttaacactactgaactgtatgctcaaaaataattaagatggtaaactttatgttatgtatatttcaccaaaattaaatgaaaatgaaattttaaaagtcaggtgtatcctcatgtattcaaataggaaaaatgctgaagatgtcaagtgaaaagaacaaatgacaaagactgtatcgtgagaacttacttgcttttgtatgcacatggccacatgtgcatagaaaacattttaggggaatgtaaatggaatattctgtaatatttgaatgatttaatgggggcatagattatcttactaaacagaaagtagattaaaaagaagctcagggaagattgtaagaatgacagaaagttgataaaaagtattatcccatatatgagataagaatcatggctgcactttaaaaagtcaagctccatgaagaccttttagcaattattataatttacttgcagatgcaatgctgctcatgctaagaggtgatcaatcaatcctttgttaaaataactagcttcctgaagttctagggaaaatccacagttgggactaaacctaaggaggtgcaaatctaaaggagtgtcaaaaactcaataatggcagcaaccccatgtgaatcgacagttcctcctggttatttgaaaacagggcagtcagcttcacctcacacctctctcaatgaatcactggagtttctgtcaggaaggggacctggaacacaattaacatgactttcactctccttgcaactgggtttactgatcctctccttccacaagaatggtgggctgcaggcgcacaaatgtaaccccataccttgggcaccaggtatagattaatacactaggattctagctccactttggaatgaaagggaccatggcttattatactgaaaaaaaatcaagaagaacacacttacattatttcaaattccatatggccctgtattattgaaactttcagtaattggctttaggcaaaaatgatgaagatggaaagaacaaaggaataaaaggacatgggaaaaataactaagaaaagagatacatggtgagtcagagagatgctctgtatccattgtggggaaaggcagacatgacttccctgcagggatgctgataactgagtgagaagatggcagagagtagacaacatgataggaaggaggtctgagacaggaaataaggaaggggagtcacttgaccctttccatatggtcatcttgcaattcaattcaacatttattgggcatcaagtgtagggcaaattcagtgccataccaagagatacatacatacacaatttacatgttgccttcaaggaattttcaattctggctttagatagaatctttaggctaaaaactgtgagaagactaatcaggtcaggagaggtgaggttgtacattccaagaaggcaagccatggccttgcttgaaatgctatttgccatttggctagtgatactgaagcacattacaaaagtaagcccatcctaagctctttcatataaaggatagatttttaaagattctgtattctggaatgtggagatatttggggggagaaggaggagaaagggaagggaatacaatgatcaggcattactgagatcattagattggataatctaaatggaaaatctatgcttctcagccccagtggggccactgcttccccagacttctggctcaagcactgaacattttctgagaggacagcctaagacctggatcaacagctttgaggctgaggtccagggcagccggggtagtgtttactctacatgctgggcagcccgtctgaaagctggggattaccttttcagttttcttacttgccaccagtcagggaagtcttcccttggaatatactgcatgttgaagtttggctgggagccatagattttctttgagttgctgtcatattgcgctagactggtacttgaagatggtggctgggagccatagtttttctttgattcgttgtcatattgggctagagtggtacttgaagatggtgcatccattttgagaacaggaactgcccgaggtatcttcagctttggaggggaagaaattttaatacatcacagtgaggcacagattcaggtaccgggaactgacaattaagtatatcagctcttgaaagagagagaaaggaaggagagaagggagggaggaagggagggaggaagggaggaaggaaagaggcaaggaaagaagaaaggtatgagcaaggatatggagcaactggaactcatattttgctggttggagtatgaagtggtacaacaacattggaaatttgtttgcagaatctagtaaatctggatgtgcatctggtcataccctatgactcataaattttacgtatatatacacaacagggaaaccaaaagacctgtaaaagaatgttcatagcagaattattttaattagataaaaactagaaactgcctacatgcccatcaagaggataaaataataaacatgttgtggttgttgaaggctgaaaaatggtctcccgaatatgttcatgtcctaattcccagaacctgtgaataccttacgtggtaaaagagactttgcagattgtaattaaggatttcgagatggggcaactattctggattttgtgggggtgggcctgatgtaatcaaaatggcctttataaaagggagataaggtggtcagagattagagagctgtgaggatggaagcagaggttagagagtagaggtcctgcaaccttctggttctgcagacaggggaaggagccaggaaccaataaatgtaagtaccccttaggaactggaaaatgcaaggaaacagaatcttccctaaagcttccagaggaaacacagctgtgcagacccattttagacatatgatcttccaaactataagagaataaatgtatgttgctttaagccactaaatttgtgtagtaatttgttagaacagtaataagaaaccaaaacagttgtatattcacacaactgaattctatacaatgatgacggtaaatataatcacatactaaaaaaataagcgaatctcacaaattgaagaaaagaagacacacaataaatgactacacactttataattccagtcatataaaagtcaaaatcaggcaaacaatctatgatgttataaatcaggttagcaactacttttggagggtagtagtgactgaaagggaacatcagggtgtccctggggttctgtgatgttgtgtttctttatctgagggtggattatacagaattgttcagtttgtgaaaattcatcaagctgaacacttaggatttgtgtgcttttctatgttaatgttatatttcagttaaaaattcacaatatgtgtgtgtgtgtctgtgtgtgcgtgtgtgacagaaagagatgcatttaatatttttttcatgctatattttagtttttcagaaatttcatttacttggagttaaggctttgacattctggaaattttaattatgcaggacagtgttatttatgagctgtgaagtgggttatgtccaaagaaaaatgcactaggaagttccttaaatggaaatcatttaaggaaatctctttgaacagaacaactagagagctgatttttatttcctccattcagaaaataagccctctactgaatactctgataaaaaacaaaacaaagaaaaacatactttacatgataccctctgatccctggttcatttgggaagcttataatcaaaacaggcttatgaacccttagccactgatttgcctagctgataattttcaatatattggccaaaaagttccctgctggagactagatgcaatggtttctggctcttaattatcttctgctcttagacttgtcaaaacaaactttattggtagttcatcctttttaaagtgcagaattcagtgacttttgtaaatttatcaacttgtataactatcaccacagcccagtcttcccatcactccaataagatccttcatacctatttgcagtcaattctcatccctactctggtcccaggcaaccactgatcaccctttgtttctatagatttgcctaatctgggcgtttctcctaggtgttttagagggccatacttttcaagcttcaggactgaaacatattttcaaacccaaacagcaatgttgctaataacaaagtaaaactcctaacgattataattgtttcctagggctaccatgacaaattgctacaagtttggtggattagaaccacaaaaatttattgtttcacagttctagaggctagaagtcaaaatcaacatttcagcattgccatacttattctgaagcctctaggataaaatctttccttgcctcttccagcttctggtggttccaagttgctatggtttggatattcgacccctccaaattttatgttgatgtgtgatctccagtgttggaggtagagcttaatgggaggtatttgggtcataggggcgaatccctcattaatggcctagtgccattctcatggtagtgaatgagttctcactctatgatttcccatgagagctggttgttgaaaagagcctgctatctcccttccctctctcttgtttcttctctctcaccatacaatttctgcacacactagctccccttcccttctgccatgagtggaagcagcctaaggccctcatcagatgccgatgccagtgctatgctacttgtacagcctgcagaacggtgagctaaataaacctcttttctttatcaattacccagcctcaggtattccttagtagagcacaaatggactaagaaacaggtgttccttggcctgtggtaacataactccaatctcttcctaggtcttcacatggccttctcctctgtgtatctctctgtgtcctctcatcttactagtcattggatttatggcccactctcatctttcgtgtgatttcatcttgatctttaactaattacatctgcaaagatcctagttccaataaggtcacattctgaggttctacatggatatgaaatttgggggacattattcagtctactacaataatggattatttttgtctttcatgttgattcatgaaaattatgtggcttttacttagaaagagagaagtgtgtgcgaatgtgtgtagctgaaaataagtacccaagacaccatagatccaatagatgggcggagatgtccccttgctttcaggtgaacttccagagaaccaatgggaagccattttttgtgtatcctggcatttgaggaggctcacacctgcctgggaaccctaaaaacagaaggctgtggctatgaacccaagagagtagagcaaaaagacagcagtagcctgtacttcgagagagagctacatacagcttaattaaccagagcaatcaaggttttgtttgttttcttataggccaactccactcttgggaaaaagttggctgacagccatcttgaaagattgagggctgaaaatccaagaactgaggatcaagatctctcccctgtcataaaactacatatggatctgcccttcagtaggaaattcctaaaagtctcccatgagataaagaatcagtgctggaaaactcactccgataccaccaccaccaaatcatgatagaaacagctatgtgtgtctttttttaattagacctcatcttccttggaactaactctgaaagggccatgaatctcagcccccccaaaatccctccccaaaagcatgctgccaggtgatgcaggcccaagctaggtgacagatgtttaacttggaatgatgtttgcagtcatgtgataataacattggatggaacaattcagaggctgttcttatgattacaagtaatggggacatttttatcatttgagaatgactgcaaaactatggaatttggcaaagactttatttggaagcagggaagaaagcccactgaatagctttgaagggataatggagggaaagaattatgttgttttctgcttttgtcctatagagtttcatttcaacaccaggatacttccacaaagcagtcttggccatgttgatggtaaggaaagaatgacagctaataacagctgcctgttatgtgtgatgccatcttaaggacatctcccgcatgcacccattttttctttttttttttttggtgactatttatgggcttactggctaggaaaagacacaacaatgaaa

SEQ ID NO: 94 - protein, 805 aa, synthetic construct [sequence omitted]

SEQ ID NO: 95 - DNA, 20 bp, synthetic construct
  misc_feature 1..20: note = Primer 2019-nCoV_N1 Forward
  source 1..20: mol_type = other DNA
gaccccaaaatcagcgaaat

SEQ ID NO: 96 - DNA, 24 bp, synthetic construct
  misc_feature 1..24: note = Primer 2019-nCoV_N1 Reverse
  source 1..24: mol_type = other DNA
tctggttactgccagttgaatctg